(19) United States
(12) Patent Application Publication
(10) Pub. No.: US 2026/0124618 A1
(43) Pub. Date: May 7, 2026

(54) ELECTRONIC DEVICES AND METHODS FOR SCALABLE CELLULAR ENGINEERING AND SCREENING

(71) Applicant: AVERY DIGITAL DATA, INC., San Diego, CA (US)

(72) Inventors: Barry MERRIMAN, San Diego, CA (US); Ryan DE RIDDER, San Diego, CA (US); Matthew T. HOLDEN, San Diego, CA (US); Kyle T. GUSTAFSON, San Diego, CA (US)

(21) Appl. No.: 18/863,970

(22) PCT Filed: May 10, 2023
(86) PCT No.: PCT/US2023/021762

§ 371 Date: Nov. 7, 2024

Related U.S. Application Data
(60) Provisional application No. 63/340,263, filed on May 10, 2022.

(51) Int. Cl. B01L 3/00; C12M 1/42

(52) U.S. Cl. CPC B01L 3/502761; B01L 3/50273; C12M 35/02; B01L 2200/0621; B01L 2200/0647; B01L 2200/16; B01L 2400/0427

(57) ABSTRACT

Methods and systems are provided for a microfluidic electrowetting array device that is integrated for material transfer with a second chip device. The integration for material transfer is configured to transfer one or more materials selected from the group consisting of: solutions, nucleic acid payloads, chemical reagents, and cells.

### CROSS-REFERENCE TO RELATED APPLICATIONS

This Application claims priority to U.S. Provisional Application No. 63/340,263, filed May 10, 2022, entitled “DEVICES AND METHODS FOR SCALABLE CELL PACKAGING AND SCREENING IN SYNTHETIC BIOLOGY.” The disclosure of the foregoing application is incorporated herein by reference in its entirety.

### FIELD

This disclosure is in the field of bioengineering. In particular, this disclosure describes microfluidic devices for the packaging of DNA payloads into cells and functional screening of cells, the use of semiconductor chips for such devices, and methods for using these in synthetic biology.

### BACKGROUND

### Cellular Engineering for Synthetic Biology

The field of Synthetic Biology is broadly concerned with engineering biological processes to achieve desired functional endpoints. One major area of focus is in modifying the DNA of cellular organisms as a means of engineering their functional properties. In particular, this includes the need to synthesize DNA sequences that correspond to individual genes, sets of genes, and larger genomic DNA constructs such as plasmids or chromosomal elements, up to the extremes of entire genomes of artificial organisms.

### Cell Packaging

One important component of such DNA engineering is a means of packaging DNA payloads into various types of cellular carriers. A large variety of methods have been developed to deliver various types of DNA payloads into various types of cells. Such methods are typically referred to as transformation, in the case of non-animal cells, such as bacteria, and transfection in animal cells. Transformation and transfection methods generally rely on means of making the cell wall permeable so that DNA payloads can pass through the cell wall. These methods may involve exposing the cells to various chemicals, enzymes, or physical processes to create such permeability. These methods may rely on biological processes, such as virus mediated transfer (known as transduction) or bacteria mediated transfer (such as agrobacterium) of the payload into the cell, or transfer through merging the cell with a lipid vesicle (liposome) carrying the payload. Among chemical methods, common ones include exposure to solutions containing divalent cations (such as calcium chloride), cationic polymers, or calcium phosphate. Among physical methods, the most common method is electroporation, in which a high voltage applied to a cell suspension causes transient pore formations on the cell walls, allowing DNA entry. Other physical methods include sonication (sonoporation), direct micro-injection using micron-scale hollow needles or injectors, or mechanoporation techniques that rely on contact with patterned structures or fluidic shear to induce pore formation through mechanical deformation. The preferred methods vary with the type of cells being targeted, such as for bacteria, yeast, plants, fungus, or animal cells, or for loading payloads into viral particles.

### Cell Screening

Another major aspect of cellular engineering is to screen the modified cells for the functional properties of interest. Screening methods are highly diverse, depending on the property in question, and many such methods have been developed. Such methods may require growing the modified cell. Such methods may require exposing the modified cell to some chemical factor or factors and observing the response or chemical products produced. In some cases, the modified cell may be expressing an engineered protein of interest, and this protein may be isolated, purified and tested in some form of activity assay.

### Electronic Microfluidic Devices

Microfluidics is a highly developed area, in which various physical means are used to manipulate microscopic amounts of fluid, for the purpose of miniaturizing and automating a great variety of wet-lab procedures. Such devices have been devised to move around droplets of fluid, and to act on them in various ways, such as merging or splitting droplets. Some of these devices are controlled electronically. Of particular interest are what are known as electrowetting devices, which use locally applied voltages to change the surface tension under a droplet, and thereby drive its motion on a surface as the means of motion control. Such methods of electrical droplet motion control are also broadly referred to as Digital Microfluidics (DMF). This may be carried out on a surface with bare metal electrodes for applying the requisite voltages, or with such electrodes covered by an insulating (dielectric) surface, in which case it is known as EWOD (electrowetting on dielectric). Such electrowetting devices for manipulating fluid droplets are also commonly known as digital microfluidic devices.

### SUMMARY

It is an object of this disclosure to disclose microfluidic electrowetting (EWOD) devices for the packaging of DNA payloads into cells and for subsequent functional screening of these cells and derived cells, for the applications of synthetic biology and more broadly bioengineering. Without limiting the foregoing, it is also contemplated that the disclosed EWOD devices can be used for non-DNA payload delivery and manipulations, such as feeding cells, growing the cells into organoids, and drug dosage profiling.

It is an object of this disclosure to disclose EWOD devices that can package DNA payloads into cells, where such payloads may comprise oligonucleotides, 20-200 bases in length, or longer DNA segments, up to 1 kilo-base (kb) in length, or up to 10 kb in length, or up to 100 kb in length, or up to 1 Mega-base (Mb) in length, or 10 Mb on length, or 100 Mb in length, or more.

It is an object of this disclosure to disclose EWOD devices in which a single such DNA molecule can be packaged into a single cell target, or in which a multiplicity of such molecules can be packaged into a single cell target.

It is an object of this disclosure to disclose EWOD devices that can perform these packaging operations in highly parallel fashion, such that up to 1000 cells may be packaged in parallel with respective distinct payloads, or up to 10,000 cells, or up to 100,000 cells, or up to 1 million cells, or up to 10 million cells, or more. In some embodiments, EWOD devices may have up to 1,000 electrodes, up to 10,000 electrodes, up to 100,000 electrodes, up to 1 million electrodes, up to 10 million electrodes, or more.

It is an object of this disclosure to disclose EWOD devices that can perform serial packaging operations on a cell, so that cells may be packaged with a multiplicity of payloads. This includes methods of combinatorial packaging, in which one or more payloads are selected from sets of available payloads, such that many different sub-selections of available payloads may be realized in the cells.

It is an object of this disclosure to disclose EWOD devices that can perform functional testing of the resulting engineered cells, or of cells subsequently grown from these cells, in a highly parallel fashion. This includes screening up to 1000 cells, or up to 10,000 cells, or up to 100,000 cells, or up to 1 million cells, or up to 10 million cells, or more. Such cells may be screened for a single assay, or multiple assays. It is the object of this disclosure to disclose EWOD devices that can perform tests of cell viability, tests for the presence of biomarkers, tests for biosynthesis of target compounds, tests for protein or enzyme function, tests for the response to various external factors, tests for cell differentiation to target types, or to test for cell interactions with other cells, ligands, factors, or antigens.

It is an object of this disclosure to disclose Complementary Metal Oxide Semiconductor (CMOS) chips devices that comprise the EWOD devices above, and which can apply the electronic control of the packaging and screening operations. This has the advantage the CMOS chips enjoy of the greatest existing manufacturing base among all types of semiconductor chips, and the greatest capacity for production and low-cost mass manufacturing, providing for both the fundamental cost reductions for deploying the EWOD devices and methods disclosed, as well as scalability of the number and complexity of such devices that can be implemented on a chip, or per square millimeter of chip area.

It is an object of this disclosure to disclose methods of monitoring and checking the processes for packaging and screening carried out on the EWOD devices, through various sensor modalities built into the devices. This has the advantage that it can be known whether the steps of packaging and screening have been properly performed.

It is an object of this disclosure to disclose systems for cell packaging and functional screening, based on EWOD chip devices.

It is an object of this disclosure to disclose methods of delivering DNA or mRNA payloads into cell-free expression droplets, based on EWOD chip devices, and performing cell free protein expression and functional screening of the resulting proteins, on such EWOD devices.

It is an object of this disclosure to disclose compositions, devices, methods, and systems for the packaging of DNA payloads into cells, and for functional testing of such cells, for applications in Synthetic Biology.

In an aspect, a method of delivering DNA payloads into cell packages is provided. The method comprises providing a first set of one or more droplets on a first portion of a microfluidic electrowetting array device, wherein each droplet comprises one or more DNA payloads. The method also comprises providing a second set of one or more droplets on a second portion of the microfluidic electrowetting array device, wherein each droplet of the second array of droplets comprises one or more cell packages. The method also comprises moving each drop of the first set of one or more droplets into proximity with a corresponding droplet of the second set of one or more droplets. Additionally, the method comprises merging each droplet of the first set of one or more droplets with its corresponding droplet of the second set of one or more droplets, thereby generating a set of merged droplets that each comprise one or more DNA payloads and one or more cell packages. The method also comprises performing an insertion process so that at least one DNA payload within each merged droplet is functionally integrated with at least one cell package within the same merged droplet.

In some embodiments, the one or more DNA payloads comprise at least two DNA payloads having a same composition. In some embodiments, the one or more DNA payloads comprise at least two DNA payloads having a first composition and a second, distinct composition. In some embodiments, the one or more DNA payloads comprise at least two DNA payloads having a first composition and at least one DNA payload having a second, distinct composition. In some embodiments, the microfluidic electrowetting array device is configured to merge droplets on a massively parallel scale. In some embodiments, the microfluidic electrowetting array device is configured to merge, in parallel between the first portion of the microfluidic electrowetting array device and the second portion of the microfluidic electrowetting array device, droplets at a scale of at least 100 droplets; at least 1,000 droplets; at least 10,000 droplets; or more than 10,000 droplets. In some embodiments, the microfluidic electrowetting array device is configured to merge, in parallel between the first portion of the microfluidic electrowetting array device and the second portion of the microfluidic electrowetting array device, droplets at a scale of at least 100,000 droplets; at least 1,000,000 droplets; at least 10,000,000 droplets; or more than 10,000,000 droplets.

In some embodiments, the insertion process comprises chemo-poration. In some embodiments, the insertion process comprises electro-poration. In some embodiments, the merged droplet comprises one or more reagents for cell-free expression in a cell-free environment.

In some embodiments, the insertion process comprises a mechanical injection. In some embodiments, the mechanical injection is a micro-injection. In some embodiments, the mechanical injection is a nano-injection. In some embodiments, wherein the insertion process comprises using a positioner device to precisely position a cell within its merged droplet. In some embodiments, the positioner device uses mechanical forces. In some embodiments, the positioner device uses electric forces. In some embodiments, the positioner device uses magnetic forces. In some embodiments, the positioner device uses magnetic forces to mechanically influence beads attached to a cell. In some embodiments, the positioner device uses dielectrophoresis forces.

In some embodiments, the method further comprises assessing the results of DNA payload activity after the insertion method has been completed, wherein the assessing comprises a least one process selected from the group consisting of: chemical stimulation, thermal stimulation, actuation, using off-array optical sensing, using one or more external sensors placed in physical contact with the microfluidic electrowetting array device, and sensing directly on the microfluidic electrowetting array device.

In another aspect, a method of using microfluidic electrowetting devices is provided. The method comprises delivering a DNA payload into a cell, wherein the delivering is through a process selected from the group consisting of: chemo-poration, electro-portion, or mechanical injection. The method also comprises performing functional screening of the resulting delivery of the DNA payload into the cell by using a fluorescent reporter assay. Additionally, the method comprises detecting a signal that results from the fluorescent reporter assay using at least one sensor selected from the group consisting of: on-device photo-sensors, or an off-device optical system, wherein the DNA payload is selected from the group consisting of: a single gene, a gene set within one long DNA molecule, an artificial chromosome within one DNA molecule, a payload that comprises a collection of multiple DNA molecules such as guide RNA's for CRISPR genome editing, or the oligo set for the process of genome engineering.

In another aspect, a system is provided that comprises a microfluidic electrowetting array device that is integrated for material transfer with a second chip device. In some embodiments, the second chip device is a printed circuit board (PCB) chip. In some embodiments, the integration for material transfer is configured to transfer one or more materials selected from the group consisting of: solutions, DNA payloads, chemical reagents, and cells. In some embodiments, the integration for material transfer is configured for transferring materials from the microfluidic electrowetting array device to the second chip device. In some embodiments, the integration for material transfer is configured for transferring materials from the second chip device to the microfluidic electrowetting device. In some embodiments, the integration for material transfer is configured for transferring materials iteratively between the microfluidic electrowetting array device and the second chip device. In some embodiments, the second chip device is a nanochannel chip device. In some embodiments, the second chip device is a DNA oligonucleotide synthesis and/or assembly device. In some embodiments, the system further comprises a third chip device that is integrated for material transfer with the second chip device and the microfluidic electrowetting array device. In some embodiments, the integration for material transfer is configured for transferring materials iteratively between the microfluidic electrowetting array device, the second chip device, and the third chip device. In some embodiments, the system further comprises a fourth chip device that is integrated for material transfer with the second chip device and the microfluidic electrowetting array device. In some embodiments, the second chip device is a nanochannel chip device and wherein the third chip device is a DNA oligonucleotide synthesis and/or assembly device.

In another aspect, a system for cellular engineering is provided. The system comprises a plurality of chip devices. The system also comprises a microfluidic electrowetting array device that is fluidly integrated with one or more of the plurality of chip devices.

In a further aspect, another system for cellular engineering is provided. The system comprises a plurality of chip devices. Additionally, the system comprises a microfluidic electrowetting array device that is fluidly integrated for material transfer with one or more of the plurality of chip devices.

In some embodiments of a system for cellular engineering, fluidic integration comprises the use of a microfluidic electrowetting array device having a second surface so as to enable fluid transfers between the plurality of chip devices. In some embodiments of a system for cellular engineering, fluidic integration comprises the use of a microfluidic electrowetting array device to enable fluid transfers between the plurality of chip devices.

In some embodiments of systems provided herein, the microfluidic electrowetting array device is implemented as a thin film transistor chip device. In some embodiments of methods provided herein, the microfluidic electrowetting array device is implemented as a thin film transistor chip device. In some embodiments of systems provided herein, the microfluidic electrowetting array device is implemented as a CMOS chip device. In some embodiments of methods provided herein, the microfluidic electrowetting array device is implemented as a CMOS chip device.

In another aspect, a system for cellular engineering is provided. The system comprises a plurality of chip devices, wherein the plurality of chip devices perform one or more process of DNA synthesis, assembly, insertion processes, and functional testing, wherein one or more of the plurality of chip devices optionally comprises a microfluidic electrowetting array device. Additionally, the system comprises a microfluidic electrowetting array device for material transfer. Additionally, the system comprises an external optical system. In some embodiments, the system further comprises at least one microfluidic electrowetting array device that performs elements of DNA assembly. In some embodiments, the system further comprises at least one microfluidic electrowetting array device that performs elements of DNA error correction.

In some embodiments of systems provided herein, the microfluidic electrowetting array device contains a number of electrodes selected from the group consisting of: about 1,000; about 10,000; about 100,000; about 1,000,000; or about 10,000,000. In some embodiments, the microfluidic electrowetting array device contains about 1,000 electrodes. In some embodiments, the microfluidic electrowetting array device contains about 10,000 electrodes. In some embodiments, the microfluidic electrowetting array device contains about 100,000 electrodes. In some embodiments, the microfluidic electrowetting array device contains about 1,000,000 electrodes. In some embodiments, the microfluidic electrowetting array device contains about 10,000,000 electrodes. In some embodiments, the microfluidic electrowetting array device contains more than 10,000,000 electrodes.

In some embodiments, the pitch of at least a portion of the electrodes is selected from the group consisting of: about 100 nm; about 1 micron; about 10 microns; about 50 microns; about 100 microns; and about 500 microns. In some embodiments, the pitch of at least a portion of the electrodes is about 100 nm. In some embodiments, the pitch of at least a portion of the electrodes is about 1 micron. In some embodiments, the pitch of at least a portion of the electrodes is about 10 microns. In some embodiments, the pitch of at least a portion of the electrodes is about 50 microns. In some embodiments, the pitch of at least a portion of the electrodes is about 100 microns. In some embodiments, the pitch of at least a portion of the electrodes is about 500 microns. In some embodiments, the pitch of at least a portion of the electrodes is more than 500 microns.

In some embodiments of systems provided herein, a droplet covers a number of electrodes selected from the group consisting of: 1 electrode; about 10 electrodes; about 100 electrodes; about 1,000 electrodes; about 10,000 electrodes; about 100,000 electrodes; or the number of electrodes within the array device. In particular, in some embodiments, a droplet covers one electrode. In some embodiments, a droplet covers about 10 electrodes. In some embodiments, a droplet covers about 100 electrodes. In some embodiments, a droplet covers about 1,000 electrodes. In some embodiments, a droplet covers about 10,000 electrodes. In some embodiments, a droplet covers about 100,000 electrodes. In some embodiments, a droplet covers substantially all electrodes on an array. In some embodiments, a droplet covers all electrodes on an array.

In another aspect, a method of delivering nucleic acid payloads into cell packages is provided. The method comprises providing a first set of one or more droplets on a first portion of a microfluidic electrowetting array device, wherein each droplet comprises one or more nucleic acid payloads. The method also comprises providing a second set of one or more droplets on a second portion of the microfluidic electrowetting array device, wherein each droplet of the second array of droplets comprises one or more cell packages. Additionally, the method comprises moving each drop of the first set of one or more droplets into proximity with a corresponding droplet of the second set of one or more droplets. The method also comprises merging each droplet of the first set of one or more droplets with its corresponding droplet of the second set of one or more droplets, thereby generating a set of merged droplets that each comprise one or more nucleic acid payloads and one or more cell packages. Further, the method comprises performing an insertion process so that at least one nucleic acid payload within each merged droplet is functionally integrated with at least one cell package within the same merged droplet.

In some embodiments, the method further comprises assessing the results of nucleic acid payload activity after the insertion method has been completed, wherein the assessing comprises a least one process selected from the group consisting of: chemical stimulation, thermal stimulation, actuation, using off-array optical sensing, using one or more external sensors placed in physical contact with the microfluidic electrowetting array device, and sensing directly on the microfluidic electrowetting array device.

In another aspect, a system is provided. The system comprises a microfluidic electrowetting array device that is integrated for material transfer with a second chip device, wherein the integration for material transfer is configured to transfer one or more materials selected from the group consisting of: solutions, nucleic acid payloads, chemical reagents, and cells.

In some embodiments of systems provided herein, the system may further comprise a microfluidic electrowetting array interface device configured to span the gap between first and second chip devices such that the droplet motion on the device transfers droplets from contact with the surface of the first chip, to contact with the surface of the second chip.

In some embodiments of systems provided herein, the systems may further comprise a microfluidic electrowetting array interface device configured to output droplets into a format of: tubes, well plates, mass spectrometry substrates, microscope substrates.

In another aspect, a system is provided. The system comprises a microfluidic electrowetting array interface device configured to span the gap between first and second chip devices such that the droplet motion on the device transfers droplets from contact with the surface of the first chip, to contact with the surface of the second chip.

In another aspect, a system is provided. The system comprises a microfluidic electrowetting array interface device configured to output droplets into a format of: tubes, well plates, mass spectrometry substrates, microscope substrates.

### BRIEF DESCRIPTION OF THE DRAWINGS

FIG. 1 shows the general form of the effective surface tension force that exists on the interface between two materials, in 3-D and 2-D views.

FIG. 2 shows the general form of the effective surface tension forces that exist between three materials and the surface tension forces on the triple junction curve, in 3-D and 2-D views.

FIG. 3 shows a local view of the contact angle that results from the balance of surface tension forces at a triple point junction where two immiscible fluid phases are in contact with a rigid planar surface.

FIG. 4 shows a local view of the contact angles for the cases of an aqueous solution on a hydrophilic and hydrophobic surface.

FIG. 5 shows an isolated aqueous droplet in equilibrium on a hydrophobic surface.

FIG. 6 shows an isolated aqueous droplet in equilibrium on a hydrophobic surface, mounted on top of a dielectric layer over an electrode, and the resulting charge distributions and forces that result from applying a positive voltage to the electrode.

FIG. 7 shows the result of an applied positive voltage driving the wetting of an isolated droplet on a hydrophobic surface.

FIG. 8 shows the result of applying a negative voltage driving the wetting of an isolated droplet on a hydrophobic surface.

FIG. 9 shows an applied positive voltage on an electrode attracting a droplet in equilibrium from an adjacent neutral electrode.

FIG. 10 shows the completed motion of a droplet from one electrode to another under the action of the applied voltage.

FIG. 11 shows an embodiment of an electrowetting configuration where droplet is confined by an upper hydrophobic surface.

FIG. 12 shows an embodiment of an electrowetting configuration where droplet is confined by an upper hydrophobic surface and the upper potential is controlled by an upper electrode.

FIG. 13 shows a top view of an array of electrodes with independent voltage control that can be used to drive droplet motion, along with a voltage timing diagram that drives the droplet along the path shown, at the time points shown.

FIG. 14 shows multiple droplet motions controlled in parallel in time, on an array of wetting control electrodes with independent voltage control.

FIG. 15 shows the process of splitting a droplet under the control of electrowetting electrodes.

FIG. 16 shows the process of merging two droplets under the control of electrowetting electrodes.

FIG. 17 shows a tube device for dispensing droplets into an electrowetting electrode array

FIG. 18 shows a tube device for outputting droplets off of an electrowetting electrode array, and the indicated process.

FIG. 19 shows an injector device for dispensing solution into a droplet under control of the electrowetting array, and the indicated injection process.

FIG. 20 shows a sampler device for extracting a sample of a droplet under control of the electrowetting array, and the indicated extraction process.

FIG. 21 shows a method of mixing the contents of a solution droplet, by repeatedly stretching and contracting the droplet on neighboring electrodes.

FIG. 21.1 shows a typical embodiment of photoelectrowetting in which light alters the capacitance of a combined insulator/semiconductor layer, thereby changing the contact angle of the droplet.

FIG. 22 shows droplets in an electrowetting device containing a cell and a DNA molecule.

FIG. 22.1 shows droplets in an electrowetting device containing multiplicities of cells and a DNA molecules.

FIG. 23 shows examples of various diverse biochemical molecules that may be contained in droplets on electrowetting arrays for the purpose of performing various processes.

FIG. 23.1 shows magnetic particles in a droplet.

FIG. 23.2 shows magnetic particles in a droplet, held in place by sufficient magnetic field upon the array as the droplet is transitioned to another site.

FIG. 23.3 shows buffer exchange using magnetic beads, which can be used for various purposes including rinsing.

FIG. 24 shows the process of electroporation, in which an applied voltage renders cells permeable for passing of DNA or other biomolecules into the cells.

FIG. 25 shows droplets configured to hold a cellular package and a DNA payload, staged for merging droplets on the electrowetting device.

FIG. 26 shows the results of merging the cell package drop and the DNA payload drop, using the electrowetting electrode to drive the droplet merger.

FIG. 27 shows the result of applying an electroporation voltage to the droplet, resulting in delivery of the DNA payload into the cellular package.

FIG. 28 shows the staging of a multiplicity of cellular packages and DNA payloads.

FIG. 29 shows the results of merging the multiple cell packages drop and the multiple DNA payloads drop, using the electrowetting electrode to drive the droplet merger.

FIG. 30 shows the result of applying an electroporation voltage to the droplet, resulting in the random delivery of the multiple DNA payloads into the multiple cellular packages.

FIG. 31 shows a configuration for packaging a DNA payload into a cell, wherein the DNA payload is presented for delivery in a vesicle. In this phase the cell and payload are positioned in adjacent droplets.

FIG. 32 shows a configuration for packaging a DNA payload into a cell, wherein the DNA payload is presented for delivery in a vesicle. In this phase the cell and payload vesicle are merged into a single droplet, using the electrowetting electrode to drive the merger.

FIG. 33 shows a configuration for packaging a DNA payload into a cell, wherein the DNA payload is presented for delivery in a vesicle. In this phase the cell and payload vesicle are fused to deliver the payload DNA into the cell, by the application of an electroporation voltage.

FIG. 33.1 shows a configuration for packaging a DNA payload into a cell, wherein the DNA payload is delivered by a viral vector.

FIG. 33.2 shows an injector device delivering DNA payloads to a cell by direct physical micro-injection.

FIG. 33.3 shows an injector device delivering DNA payloads to a cell by direct physical micro-injection, combined with a cell positioning tip to bring the cell to a defined spatial location.

FIG. 33.4 shows an injector device delivering DNA payloads to a cell by direct physical micro-injection, combined with a dielectrophoretic electrodes used to bring the cell to a defined spatial location.

FIG. 34 shows droplets containing two different reagents for a biochemical reaction A+B→C.

FIG. 35 shows the A+B reaction going forward to produce the reaction product, as a result of combining the reactant droplets on the electrowetting electrode.

FIG. 36 shows droplets containing two different reagents for a reaction, one being a cellular reactant, the other being a factor X to stimulate the cell.

FIG. 37 shows the reaction going forward to produce the cellular reaction product Cell*, activated by Factor X, as a result of combining the reactant droplets on the electrowetting electrode.

FIG. 38 shows examples where the induced cellular change from exposing the cell to a factor to activate it to Cell* may then result in cell division, or cell death, or the production of various biomolecules, which may be internal to the cell (P), on cell surface (S), or excreted from the cell (E).

FIG. 39 shows droplets containing the reactants for cell free expression: coding mRNA and ribosomes and reactants for translation and protein expression.

FIG. 40 shows the cell free protein expression reaction occurring, processing the mRNA delivered in the droplet to produce multiple copies of the encoded protein.

FIG. 40.1 shows droplets containing the reactants for cell free expression starting from a DNA payload: the other reactants now include polymerase and RNA nucleotides (rNTPs) to produce mRNA intermediates.

FIG. 40.2 shows the cell free protein expression reaction occurring, transcribing the DNA payload into multiple mRNAs, which are in turn translated to produce multiple copies of the encoded proteins.

FIG. 41 shows various possible configurations of sensor electrodes, for various possible types of sensors.

FIG. 42 shows various possible configurations of sensor electrodes, for various possible types of sensors, in side and top views.

FIG. 43 shows possible configurations of photo-detectors to detect light emission from droplets, possibly with micro-lenses to collect light.

FIG. 44 shows possible configuration of an imaging pixel array relative to droplet control electrodes for imaging droplet and cell contents with spatial resolution.

FIG. 45 shows possible locations of actuator sensors, to apply various types of actuator stimulation to the droplet.

FIG. 46 shows specific types of actuators: an electrode for heating or cooling of the droplet, and an electrochemical electrode to induce pH changes or other electrochemical stimulations.

FIG. 47 shows configurations of LED actuators, for applying light stimulation to the droplet.

FIG. 48 shows an example of an electrowetting electrode array device and method for packaging of DNA payloads into cells in a parallel, scalable array format. Shown is the process phase of staging DNA payload droplets and cell packages on the array.

FIG. 49 shows an example of an electrowetting electrode array device and method for packaging of DNA payloads into cells in a parallel, scalable array format. Shown is the process phase of merging the DNA payload droplets and cell packages droplets on the array, in parallel.

FIG. 50 shows an example of an electrowetting electrode array device and method for packaging of DNA payloads into cells in a parallel, scalable array format. Shown is the process phase of applying electroporation to the droplets, to drive the DNA payloads into the cells, in parallel across all droplets.

FIG. 51 shows an example of an electrowetting electrode array device and method for packaging of DNA payloads into cells in a parallel, scalable array format. Shown is the process that allows for staging of a series of DNA payloads, and cell droplets passing by can selectively merge to allow for combinatorial combinations of the payload options into the cells.

FIG. 52 shows an example of an electrowetting electrode array device and method for packaging of DNA payloads into cells in a parallel, scalable array format. Shown is the process that allows for various combinatoric selections of DNA payload droplets to be merged into the cell droplets, for subsequent electroporation.

FIG. 53 shows an example of an electrowetting electrode array device and method for packaging of mRNA payloads into cell-free protein expression droplets in a parallel, scalable array format. Shown is the process phase of staging mRNA payload droplets and droplets of expression reagents on the array.

FIG. 54 shows an example of an electrowetting electrode array device and method for packaging of mRNA payloads into cell-free protein expression droplets in a parallel, scalable array format. Shown is the process phase of merging the mRNA payload droplets into droplets of expression reagents on the array.

FIG. 55 shows an example of an electrowetting electrode array device and method for packaging of mRNA payloads into cell-free protein expression droplets in a parallel, scalable array format. Shown is the process phase of cell free expression occurring in the droplets to produce proteins from the respective mRNA.

FIG. 56 shows a portion of the array dedicated to droplet QC, with sensors electrodes capable of identifying mis-formed droplets, such as may be used at the location where droplets are loaded onto the array, or at other regions of the array where QC is needed. Droplets to be tested are staged in the area.

FIG. 57 shows the first step of droplet QC, where staged droplets are moved over the sensors, and sensors are used to detect well-formed droplets (highlighted in green) and mis-formed droplets (highlighted in red).

FIG. 58 shows the next step of droplet QC, mis-formed droplets are moved to a discarding area, for discarding off the array.

FIG. 59 shows the final step of droplet QC, where well-formed droplets are reorganized and staged for subsequent processing.

FIG. 60 shows an electrowetting electrode array configured for treatments, actuation and sensor of the droplets produced by payload packaging stages. Indicated are zones for addition stimulating factors to the cells (or cell-free proteins), followed by a zone with sites for actuations, and followed by a zone with sites for readout sensors. Droplets can progress through these zones, and also cycle between them, via the independent droplet motion control.

FIG. 61 shows that droplets of different sizes larger than the primary electrode size can be moved on an array of wetting control electrodes with independent voltage control, providing the ability to manipulate droplets of larger sizes on the same electrode array.

FIG. 62 Shows that droplets of different sizes larger than the primary electrode size can be used to carry larger sized cells, or a larger number of cells, for cell packaging and cell assay manipulations.

FIG. 63 shows a fluidic interface between a nanochannel device containing DNA payloads, and an EWOD device for encapsulating DNA payloads in droplets on the array, for subsequent payload processing.

FIG. 64 shows a fluidic interface between an electrophoretic device capable of transferring DNA oligos or short fragments from electrode to electrode via electrophoresis, and an EWOD device for encapsulating the DNA payloads in droplets on the array, for subsequent payload processing.

FIG. 64.1 shows a fluidic interface between a dielectrophoretic device capable of transferring cells from electrode to electrode via dielectrophoresis, and an EWOD device for encapsulating the cell payloads in droplets on the array, for subsequence payload processing.

FIG. 65 shows a bi-directional EWOD device fluidic interface, between a nanochannel device containing DNA payloads, and an Electrophoresis device containing DNA payloads, wherein the EWOD device in can extrude droplets with DNA from one side, and deliver the DNA to the other, in either direction.

FIG. 66 shows a basic CMOS embodiment of the schematic electrowetting electrode and control voltages.

FIG. 67 shows a circuit schematic for a CMOS chip embodiment of the basic electrode pixel for an EWOD array CMOS chip.

FIG. 68 shows a top-level architecture circuit for a CMOS chip embodiment of an EWOD electrode array CMOS chip.

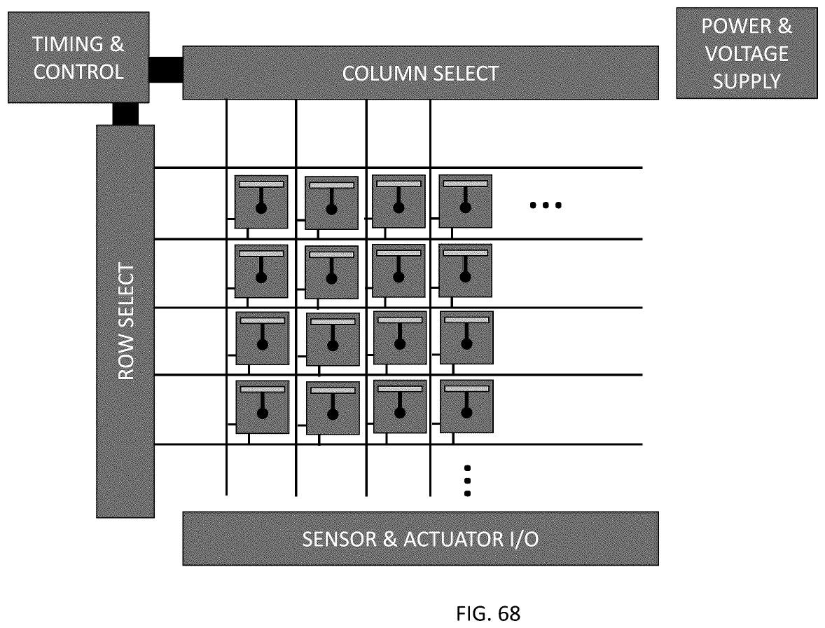

FIG. 69 shows a system architecture diagram for an EWOD CMOS-chip based device for the packaging of DNA payloads into cells, combined with subsequent functional assays on chip.

FIG. 70 shows a system architecture diagram for an embodiment of an EWOD CMOS-chip based device for the packaging of DNA payloads into cells, where the interface to the DNA source or the DNA source itself may further comprise EWOD chip devices.

FIG. 71 shows a system architecture for embodiments in which a TFT array device provides an EWOD interface device for chip-to-chip fluid transfer using droplets and allowing an imaging system to view such operations.

FIG. 72 shows various preferred embodiments using a TFT EWOD interface device.

FIG. 73 shows a preferred embodiment of how the TFT EWOD interface device overlays the CMOS chip device pixel arrays to support liquid transfer, manipulation, and I/O.

FIG. 74 shows a system architecture for embodiments in which an EWOD interface device is used to transfer solution from one device chip to another using droplet microfluidics.

FIG. 75 shows a system architecture for embodiments in which an EWOD interface device is used to fluidically connect multiple chip devices.

FIG. 76 shows various preferred embodiments using an EWOD interface device for transfer of fluids between chip devices.

FIG. 77 shows preferred embodiment of system architectures with subsystems that support various forms of electrical and fluidic of I/O.

FIG. 78 shows a preferred embodiment of a system architecture with an external imaging system.

FIG. 79 shows a preferred embodiment of a system architecture with an external imaging system, including a high-resolution imaging subsystem.

FIG. 80 shows the electrokinetic localization and transport of fluorescently labeled DNA molecules on silicon-based devices. Optical microscopy images depict the surfaces of Device 1 (panel a) and Device 2 (panel b), which were used for AC manipulation of PhiX DNA (1 kb) and DC manipulation of small oligos (15 bp), respectively. PhiX DNA localizes around (panels c and d) and ejects from (panel e) an active node in a frequency-dependent manner via AC electrokinetics. Small oligos localize at an active node (panel f) via DC electrokinetics. Changes to the applied electrode configuration in panel f alter the shape of the induced electric field, prompting a transfer of small oligos from the initial site of localization to a secondary site (panels g and h). Arrows indicate the direction of particle motion. Boxes with solid lines denote active nodes. Boxes with dashed lines depict high-impedance nodes. Unboxed electrodes are grounded.

FIG. 81 shows droplet motion via electrowetting on dielectric. A. Four droplets positioned on electrode array. B. Left and rightmost droplets move one electrode down by applying 240V. C. Left and rightmost droplets move two electrodes down by sequentially applying 240V. D. Leftmost droplet (blue) moves four electrodes to the right and rightmost droplet (yellow) moves four electrodes to the left by sequentially applying 240V.

FIG. 82 shows droplet mixing and splitting via electrowetting on dielectric.

FIG. 83 shows bulk particle buffer exchange via electrowetting on dielectric

FIG. 84 shows single particle encapsulation & manipulation via electrowetting on dielectric.

FIG. 85 shows cell-free protein expression via electrowetting on dielectric.

FIG. 86 shows cell transfection via electrowetting on dielectric.

FIG. 87 shows electroporation via electrowetting on dielectric.

FIG. 88 shows DNA assembly via electrowetting on dielectric.

FIG. 89 Shows DNA assembly and chemoporation via electrowetting on dielectric.

FIG. 90 Shows DNA error correction via electrowetting on dielectric.

### DETAILED DESCRIPTION

It is the object of this disclosure to disclose compositions, devices, methods, and systems related to using EWOD devices to load DNA payloads into cells, and to perform diverse functional testing on such cells, in a highly parallel, scalable, and cost-effective manner.

### Fundamental Electrowetting Processes

Electrowetting devices move droplets of fluid by using voltage to manipulate the surface tension forces acting on the droplet. For the purpose of disclosing such devices and methods, it is helpful to introduce the relevant physical concepts that underly their function, and the proper physical terminology and conventions for describing these phenomena.

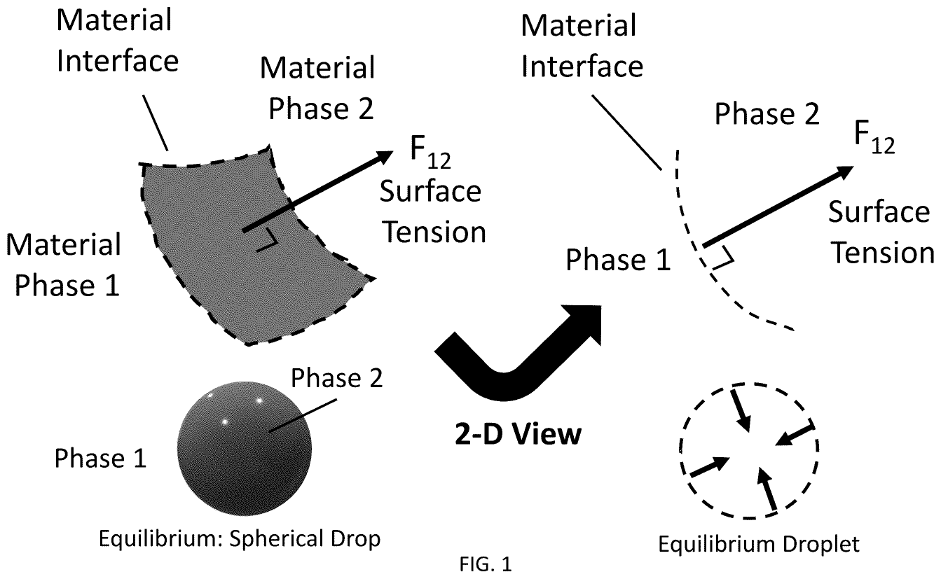

To begin, it is beneficial to describe what happens when two materials come into contact at an interface. In general, as shown in FIG. 1, when two materials that do not mix (“immiscible”) come into contact, they form a material interface, such as for air and water, or oil and water, or water and glass, which are representative of the phase boundaries that occur in EWOD devices. At such material interfaces the molecular forces within and between the materials imply that energy is required to move a molecule of a material to the interface, and therefore there is a total energy per unit area of interface contributing the total thermodynamic energy of the system. The effect of this surface energy on the dynamics of the materials is described by an effective force acting on the surface, called the “surface tension”, that is directed normal to the surface and has a magnitude equal to the energy per unit area, and is generally directed so as to reduce the local surface area if the materials were free to move under the action of this force. The work done by this force accounts for the energy changes that result from changing the interface area, and this force generally acts to reduce the interface area. In the case of immiscible fluids, if one fluid is surrounded by the other, in the absence of any other external forces this surface tension force drives the surface towards minimum area enclosing the volume, resulting in an equilibrium of a spherical droplet of one fluid, surrounded by the other, as indicated in FIG. 1. As shown at right in FIG. 1, it is convenient to depict this three-dimensional force geometry in a 2-D cross-sectional diagram, where the cross section is taken orthogonal to the surface in question, so that the material boundary can be indicated by a curve, and the surface force is normal to this curve, and directed towards the local center of curvature (the direction that would shorten the curve if it moves under the action of the force).

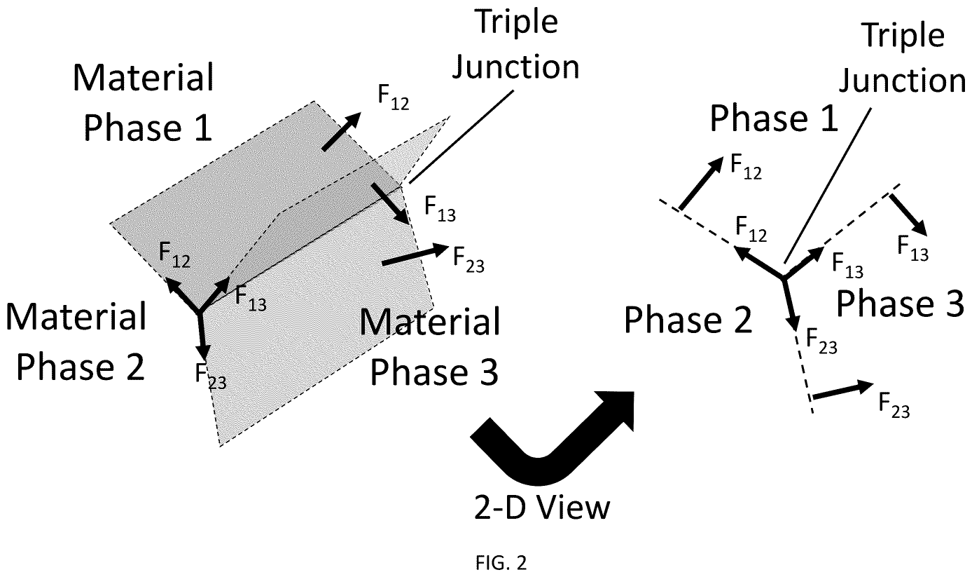

For the case of interest for EWOD devices, we must consider droplets moving on a surface, so it is necessary to consider what happens when there are three materials in contact, such as air, water and glass, or oil, water and glass. In general, as shown in FIG. 2, when three immiscible materials are in contact, there will exist a “triple junction” curve in space along which all three materials are in contact. The surface tension forces between each pair of material interfaces result in a net force per unit length on this curve, equal to the surface tension and acting in the plane of the interface and normal to the curve, as shown. This can again be more easily depicted in a 2-D cross-sectional view as shown at right in FIG. 2, where the junction becomes a “triple point”, and the three surface tension forces are shown acting on this point. In general, these three forces sum to produce a net force on this point, F=F₁₂+F₂₃+F₁₃, and for materials that are free to move, this junction point will move under the action of this force, and any other forces acting on the system. It will come to equilibrium when these forces, and any other forces present, balance to zero net force, and this balance of forces condition defines the angles between the phase boundaries at this point.

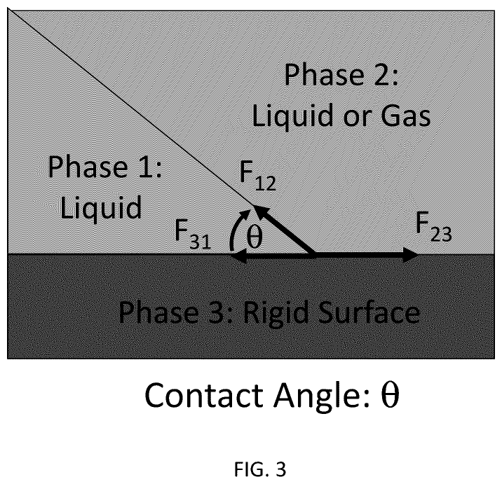

In the case of interest for describing EWOD devices, there is a droplet of one fluid, such as water, immersed in another fluid such as oil or air, and this all takes place on a rigid material (the dielectric) surface, such as a planar glass or plastic surface. The special case of this 3-phase system is depicted in FIG. 3. In this special case, near the junction the local material interfaces are nearly planar, and the net effect of the surface tensions forces present can therefore be depicted by focusing on the three surface tension forces acting on the triple point junction. Here, because the planar dielectric surface is rigid, it balances any tension forces in the vertical direction normal to the surface, so all that matters in the horizontal component of these forces. If the droplet sits on the surface in equilibrium, these three forces must sum to having zero horizontal component, and this geometry is described by a single phase angle, the “contact angle”, θ, shown in FIG. 2, which can be defined as the angle between the planar surface and the tangent to the droplet at the point of contact, and which is defined by the condition of zero horizontal force, or F₂₃=F₁₂ Cos(θ)+F₃₁. As the surface tension forces are material thermodynamic parameters (interface energies), this equation defines the contact angle.

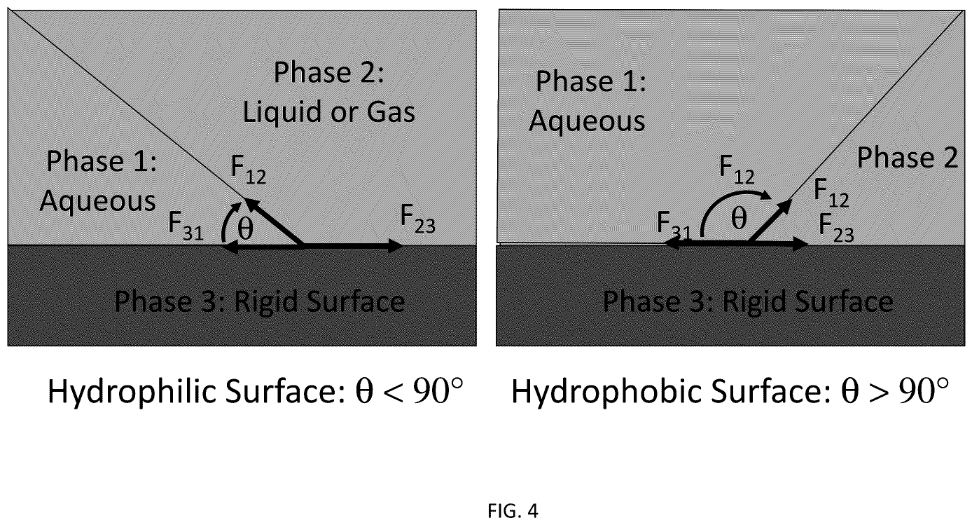

In the more specific case of interest for EWOD, as shown in FIG. 4, the droplet will be an aqueous (water-based) solution, the other phase may be an oil or gas, or other phase immiscible with water, and the dielectric surface will either be more attracted to water than to the other phase (a “hydrophilic” surface), i.e. higher surface energy, F₂₃>F₁₃, which results in the contact angle being less than 90 degrees, or the planar surface will be more attracted to the other phase than to water (a “hydrophobic” surface), i.e. lower surface energy, F₂₃<F₁₃, in which case the contact angle is greater than 90 degrees. In sufficiently hydrophilic cases, the surface energy of the water-plane interface is so low relative to the other energies that there is no equilibrium, and the aqueous drop will instead spread out as much as possible to maximize the coverage of the planar surface. In this case it is said to wet the surface, and the contact angle is effectively 0. Note that while the descriptive language (“hydrophilic” or “hydrophobic” surface) focus on the droplet and the surface, these are all properties that depend on the third (“ambient”) phase as well, as it is the balance of all three interface energies that determine the contact angle., or whether wetting occurs.

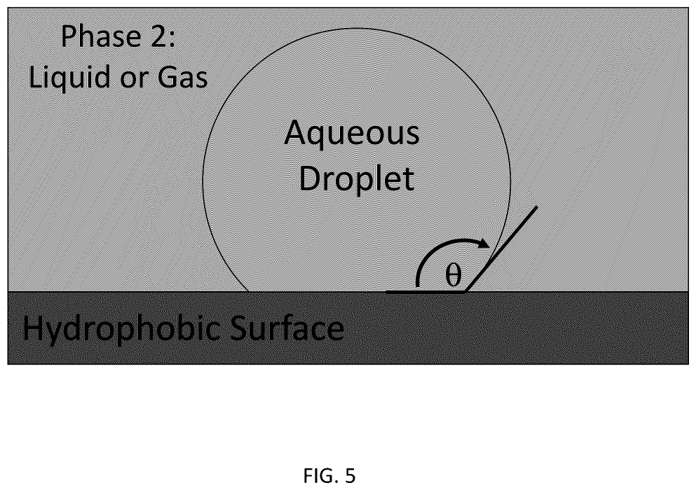

FIG. 5 shows the case typical for EWOD devices, in which there is an aqueous droplet on a native surface that is hydrophobic (relative to the other ambient phase, such as oil or air), and therefore in equilibrium the droplet “beads up” on the dielectric surface, with some contact angle greater than 90 degrees.

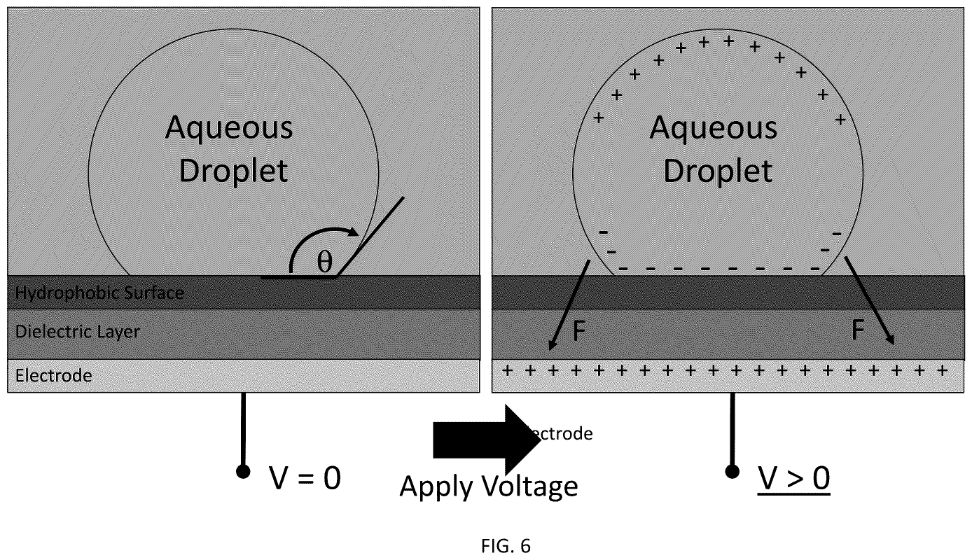

FIG. 6 shows the impact of applying a voltage to a droplet as is done in an EWOD device: the material stack under the droplet consists of the hydrophobic surface, which is on top of dielectric (insulating) layer, which in turn is on an electrode layer to which the voltage is applied. At left is shown the equilibrium in which there is no applied voltage. At right is shown the result of applying a voltage, in this case a positive voltage. As shown, the applied voltage produces a net positive charge on the electrode. The droplet is assumed to contain an aqueous solution which is conducting to some degree, such as a solution containing salts or other ions.

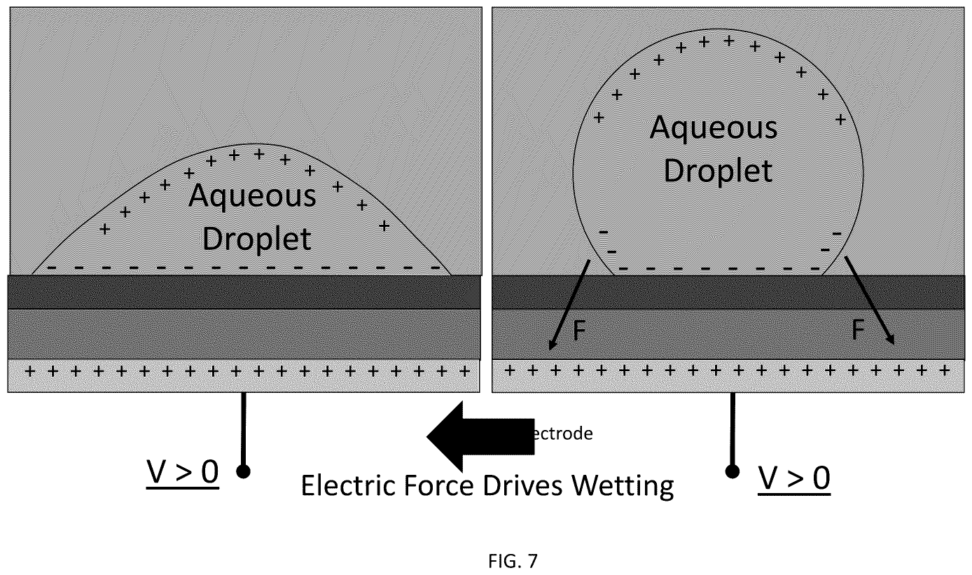

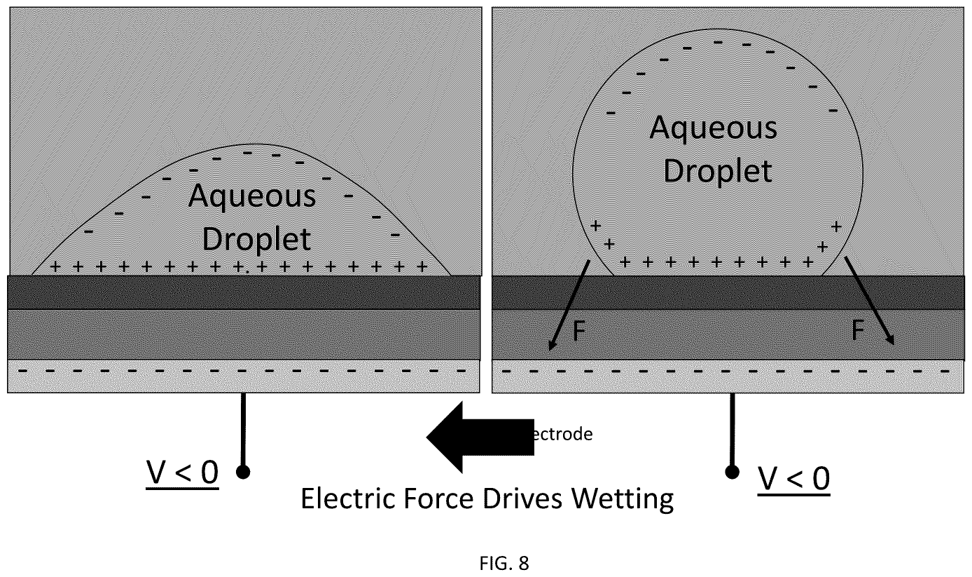

The positive charge on the electrode will attract a net negative charge to the bottom surface of the droplet, and as a result an excess of positive charge will result on the distance surface of the droplet. With this charge distribution, there is created an attractive electric force F on the negative surface. This attraction is effectively a surface tension force attracting the droplet to the material surface. As shown in the continuation in FIG. 7, the result of this electrical force is to spread the negative droplet surface over the material surface, causing the droplet to “wet” the surface, hence the name “electrowetting”. As shown in FIG. 8, the net same wetting effect results if the applied voltage on the electrode is negative instead of positive. All the signs of the charges are reversed, but the net forces driving droplet motion are the same.

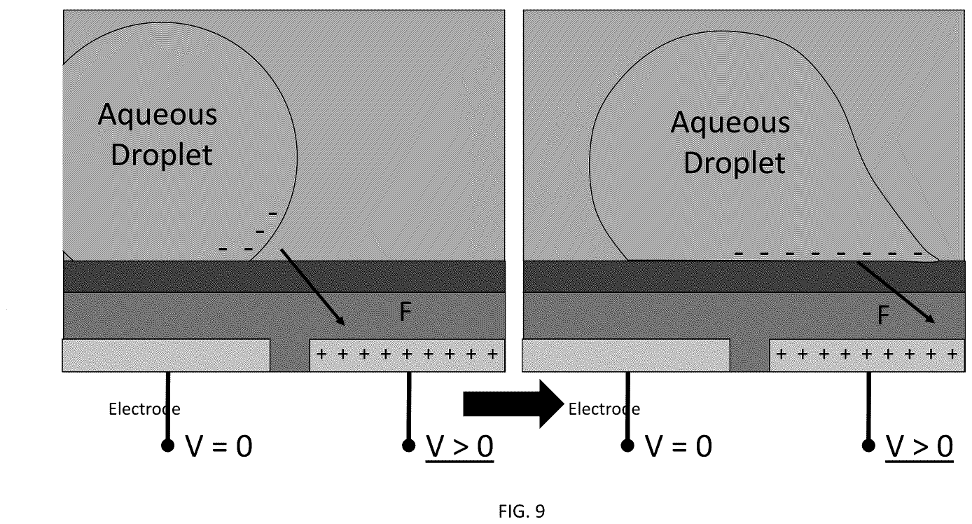

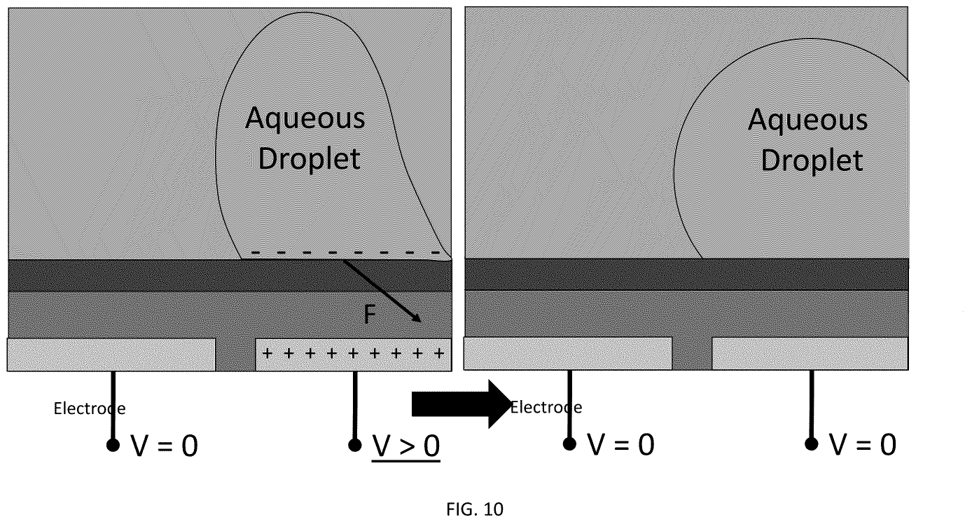

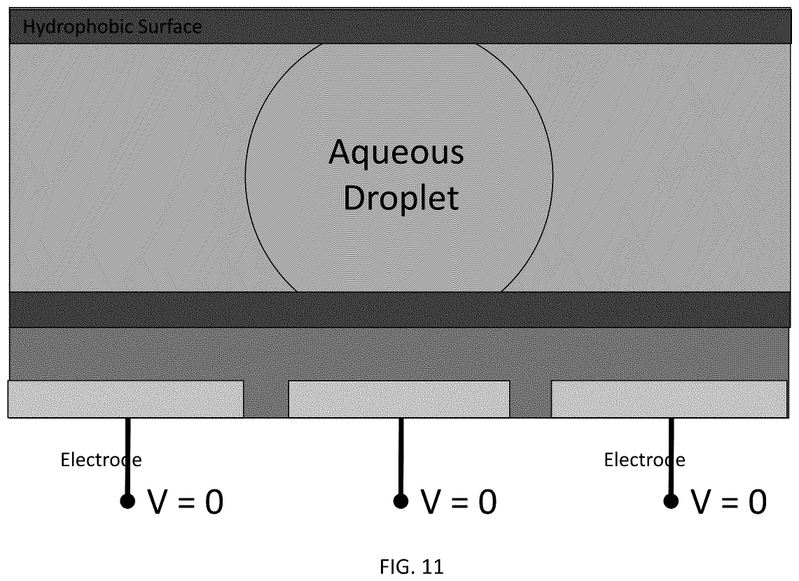

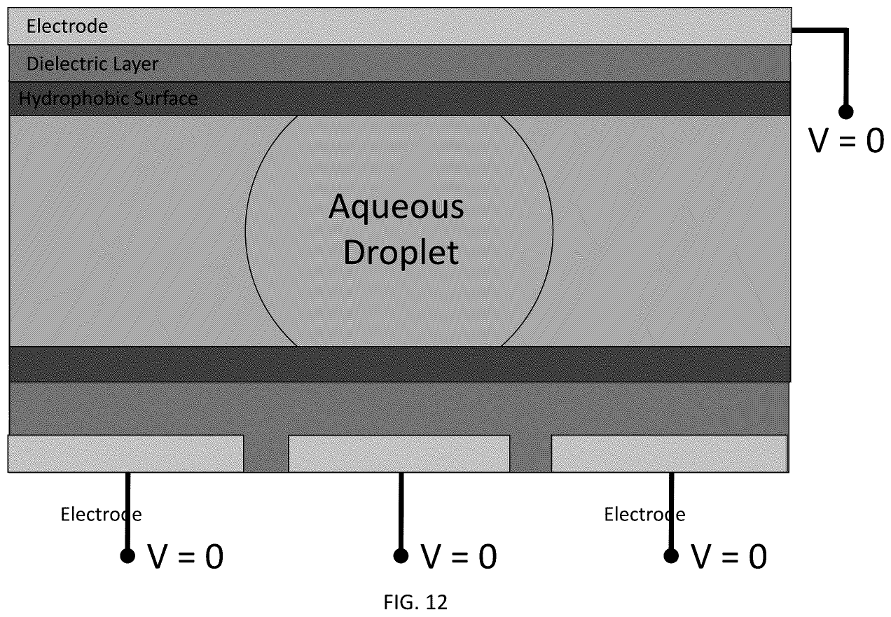

FIGS. 7 and 8 therefore show the fundamental mechanism by which an applied voltage cause the droplet to spread over the surface. FIG. 9 further shows how the multiple independent electrodes can use this effect to move the droplet in a desired direction: if the droplet sits above a neutral electrode, and voltage is applied at an adjacent electrode, as shown at left, a net attractive surface force is created biased towards the active electrode. As shown at right, this force will pull the droplet surface towards the active electrode. As indicated in FIG. 10 left, this force will pull the entire droplet over the active electrode. Then, shown at right, if the applied voltage is removed, the droplet will return to its equilibrium form. The net effect, shown in FIGS. 9 and 10, is that the droplet is moved from one electrode to the adjacent electrode through this voltage directed process. This is the fundamental motion operation of the electrowetting device. In preferred embodiments, as shown in FIG. 11, there may also be an upper hydrophobic surface to further confine the droplet. The ambient solution surrounding the aqueous droplet could be an oil, or another immiscible liquid, or air, or other gas. In preferred embodiments, as shown in FIG. 12, there may also be an upper electrode, which could in preferred embodiments be set at a neutral voltage, to further define both the geometry and electrical state of the droplet.

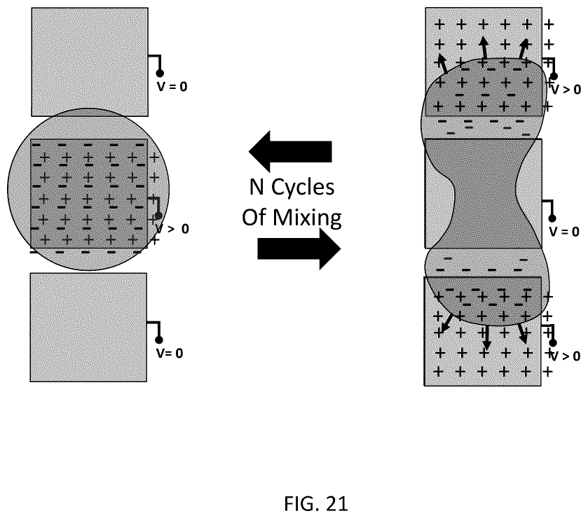

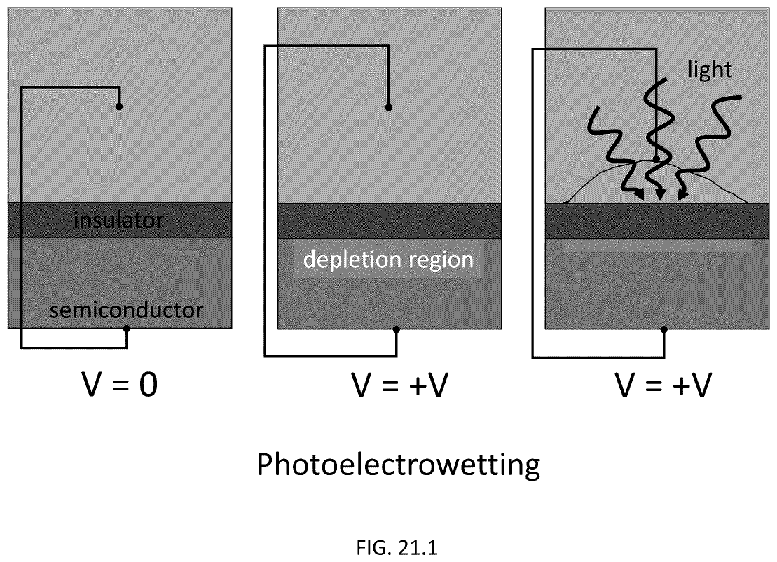

FIG. 21.1 shows a fundamental mechanism for photoelectrowetting in which applied light, and voltage are used to alter the contact angle of a droplet in a multi-phase system. A liquid-phase droplet contacts an insulator layer patterned on top of a semiconductor material. Application of a bias voltage generates a depletion region within the semiconducting material that is devoid of mobile charge carriers like electron-hole pairs. The resultant space-charge region at the interface of the insulator and semiconductor layers may be sensitive to light of energy greater than the semiconductor's bandgap energy in such a way that illumination generates electron-hole pairs in the semiconductor. The application of light thereby reduces, eliminates, or otherwise alters the depletion region, modifying the capacitance of the combined insulator and semiconductor layers. As a result, the contact angle between the droplet and the insulator layer changes. Photoelectrowetting may have the advantage of requiring less applied voltage to drive droplet motion, which may be useful for implementation of such circuits in voltage-limited devices, such as implementing such circuits in certain semiconductor integrated circuit chip devices.

Similarly, another form of electrically driven wetting phenomena that can move droplets is “electro-de-wetting”, in which the fluid droplets are configured to have a surface charge of one sign (such as by the inclusion of ionic surfactants, which are molecules with a charged head group and a hydrophobic tail, and which organize themselves in an aqueous droplet such that the head group charges populate the surface of the droplet), and then by applying the opposing charge on an electrode, it will repel the droplet surface (“de-wetting”) from the electrode, driving droplet motion. Electro-dewetting can thereby be used in the same fashion as electrowetting to move droplets. An advantage of electro-dewetting is that it can move droplets with lower applied voltages, which may be more compatible with the voltage limitations such as might arise with using semiconductor chip integrated circuits for such devices. Thus, in what follows, it is understood that EWOD encompasses such electro-dewetting devices as well.

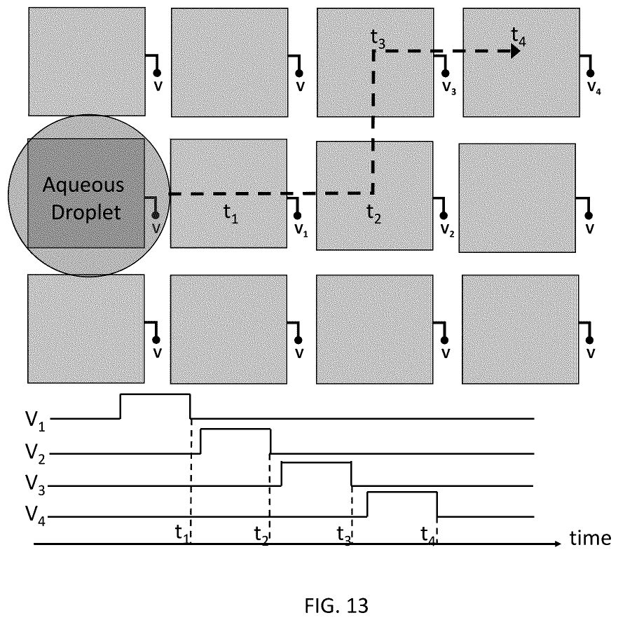

As shown in FIG. 13, in preferred embodiments there is an array of such motion control electrodes, closely spaced, and by repeating this process of applying voltages to direct the motion, over an array of electrodes, the droplet can be moved at will, passing from one electrode to an adjacent electrode under voltage direction. Shown is a trajectory of the droplet over the array (dashed line) indicating the time points at which the droplet arrives at the electrodes, and the controlling voltage required to produce this motion are indicated in the timing diagram (bottom) showing how voltage is sequentially pulsed at electrodes have control voltages v₁, v₂, v₃ and v₄.

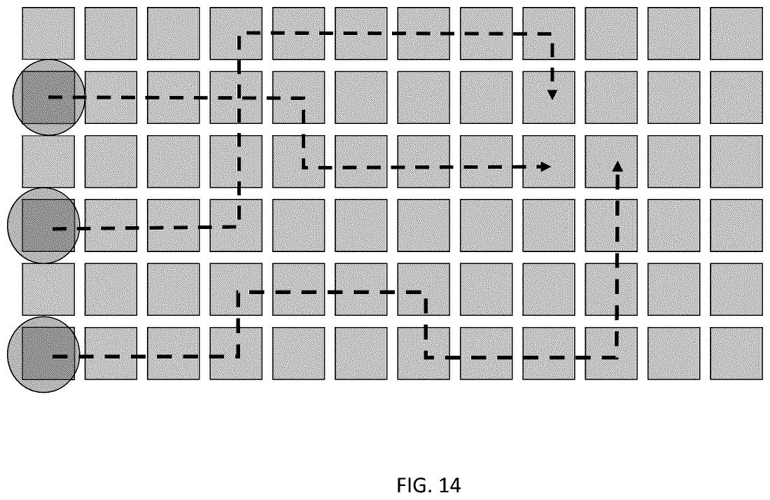

As shown in FIG. 14, in preferred embodiments droplets can be moved in parallel on such an electrode array, executing complex independent trajectories under the independent control of the indicated electrodes. In this way, on a large electrode array, many droplets can be made to execute complex motions in parallel. It is obvious from this disclosure, that many such parallel motion trajectories and timings can be implemented on such devices.

### EWOD Device Fabrication

Such EWOD electrode arrays can be implemented using any of various techniques of realizing electrodes and electrical control circuits. In preferred embodiments, it is advantageous to use methods that enable efficient design and fabrication of such circuits. In preferred embodiments, EWOD electrode arrays can be implemented as Printed Circuit Board (PCB) devices, where the electrodes and control wires to the electrodes are fabricated directly as part of the PCB device. Such devices have advantages of low cost, ease of design, simple fabrication, and broad commercial support for custom devices, but they have a limited scale (electrode density or pitch, typically limited to the ˜1 mm electrode scale) and required auxiliary switches and complex discrete control circuitry to control the electrodes, and so are typically only advantageous and practical for arrays having 10's to 100's of electrodes. In other preferred embodiments, EWOD electrode arrays can be implemented as Thin Film Transistor (TFT) devices. Such devices have the advantage that the TFTs can be integrated as control switches, thereby enabling scalable control systems that make it practical to control much larger arrays, from thousands to tens of millions of electrodes (such as are used in TFT active matrix displays), and the advantages of higher electrode density (with pitches down to 10's of microns), and unique advantages of transparent devices (enabling fully enclosed devices with imaging of the droplets through the back of the array) and low cost-large area devices (from 10's to 100's to 1000's of square centimeters), as well as options for commercial mass production by leveraging the TFT display industry. In yet other preferred embodiments, EWOD electrode arrays can be implemented as semiconductor chip integrated circuit devices, and most preferably as CMOS (Complementary Metal Oxide Semiconductor) devices. CMOS chip devices have the advantages of maximal circuit scaling, by virtue of integrated circuit technology, which can support EWOD arrays with pitch down to 1 micron or smaller. They also have the advantage of the most highly developed design and production infrastructure, which provides relatively low-cost design and production of chips, from very low production volumes (hundreds of chips) through very high volumes (billions of chips). CMOS chip devices also have the advantage that complex control circuity and diverse sensors (such as electrode current sensors, electrode impedance sensors, chemical sensors, and photo-sensors, including imagers) can be integrated into the chips, to add additional functionality and sophisticated to the EWOD arrays.

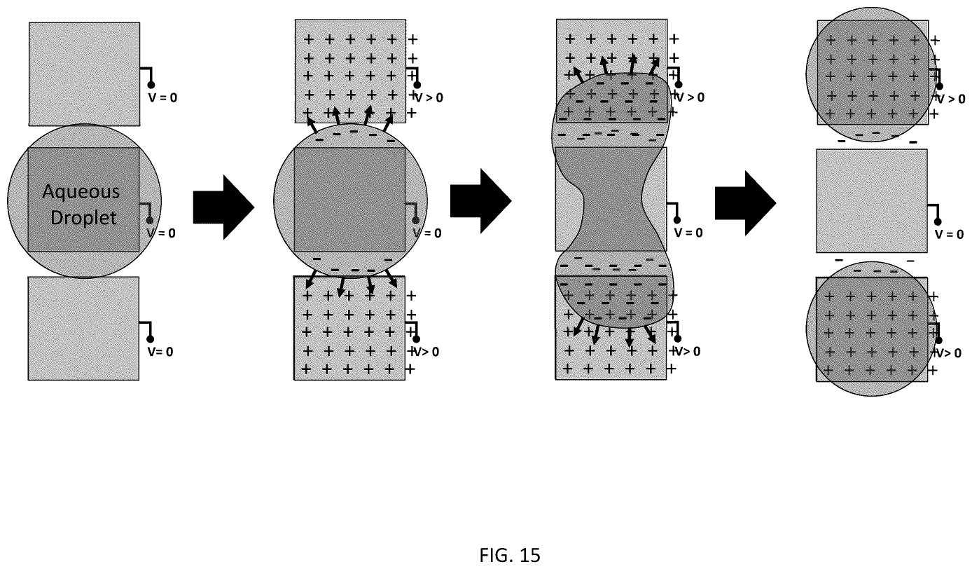

FIG. 15 shows how the electrowetting forces can be used to drive a droplet to split into two droplets, on adjacent electrodes. As shown, starting from the droplet at equilibrium (left), if positive is voltage is applied to both adjacent electrodes, the induced surface charges on the droplet create local surface forces pulling towards both electrodes, which will drive droplet motion in both directions, causing the droplet to elongate and ultimately split in two, and come to equilibrium as two droplets above the respective driving electrodes. The net effect is to split the original droplet into two droplets.

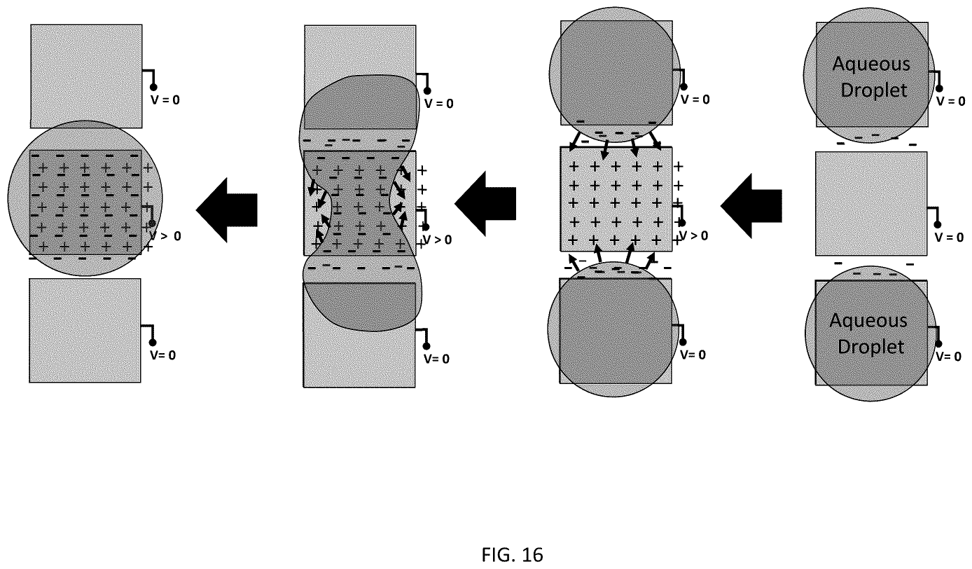

FIG. 16 shows the opposite operation, in which two droplets on neighboring electrodes (right) can be attracted to merge into one droplet on the central electrode, by applying voltage to the central electrode.

It is obvious from the disclosures in FIGS. 14, 15, and 16 that a great many complex parallel motions of droplets, including splitting and merging of droplets, can be achieved on an array of electrowetting electrodes, by using the voltage control methods disclosed, applied in concert.

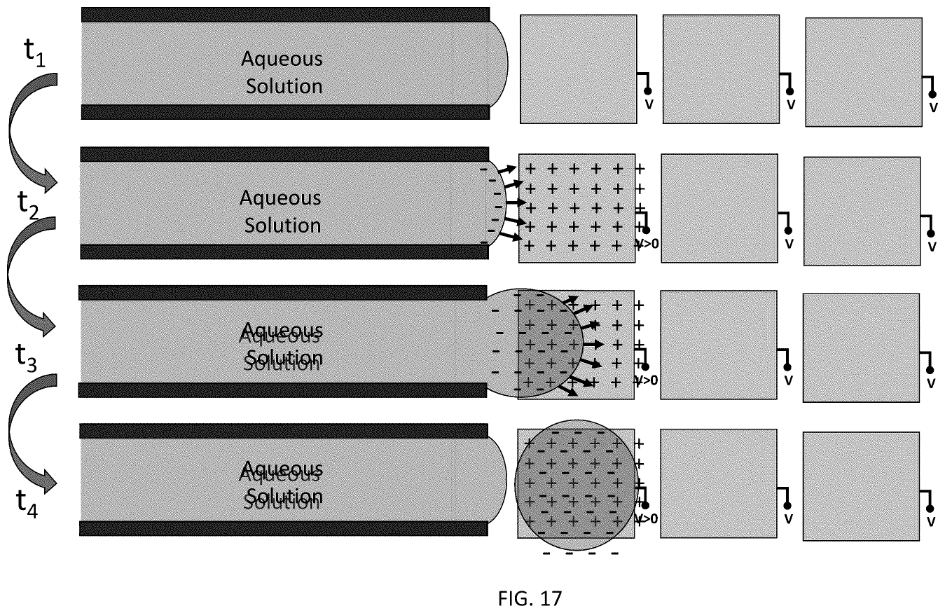

FIG. 17 shows a preferred embodiment and method for dispensing droplets onto the electrode array. At the initial time t₁, shown at top, a tube with hydrophobic walls is placed in close proximity to the electrode array, such that the aqueous solution in the tube extends outward near the first electrode. At time t₂, shown below, the first electrode is activated, creating an attractive surface force on the solution extending from the tube, as shown. At time t₃, shown below, this force has pulled the solution from the tube to wet the first electrode. At final time t₄, the droplet is fully dispensed onto the first electrode, and is ready for subsequent motion under control of the array electrodes. From this disclosure, many such similar means of dispensing droplets onto the electrode array, with diverse geometries, including allowing parallel dispensing tubes, are obvious, and all such obvious embodiments are implied by this exemplar illustrated. Other preferred embodiments could include traveling-wave electroosmosis or electrothermal pumping. This provides a means to introduce droplets to the array.

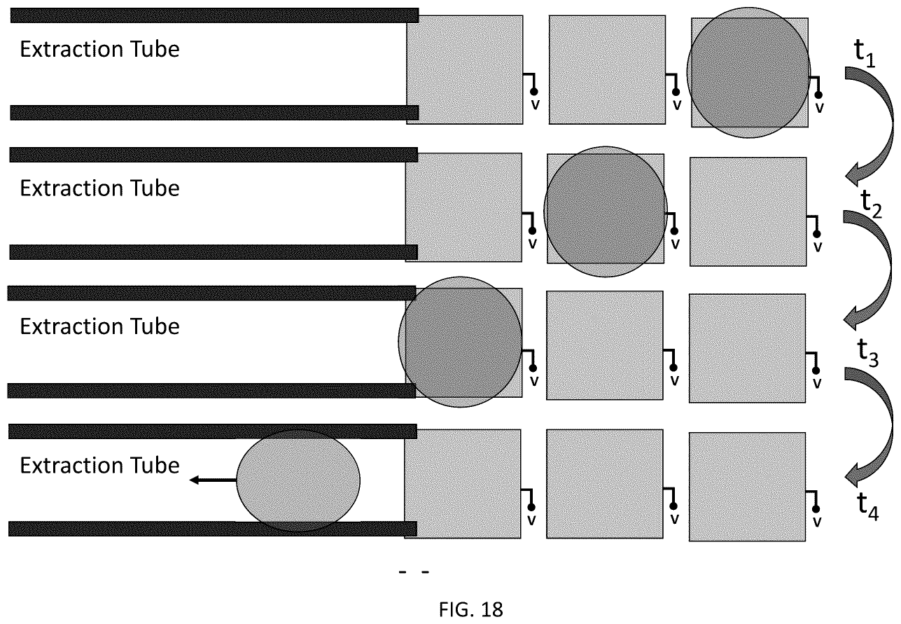

FIG. 18 shows a preferred embodiment and method for outputting droplets from the electrode array. At the initial time t₁, shown at top, a tube with hydrophobic walls is placed in close proximity to the electrode array, and a droplet is brought to a location nearby. At time t₂, shown below, the adjacent electrode is activated, moving the droplet closer to the tube intake as shown. At time t₃, shown below, the electrode adjacent to the tube is activated to move the droplet to the tube inlet. At final time t₄, a driving intake force is applied to the tube, such as by applying a voltage to the tube, so that the droplet is drawn into the outlet tube. Once in the tube, the droplet is routed away from the array, under the action of such forces. From this disclosure, many such similar means of outputting droplets from the electrode array, with diverse geometries, including allowing parallel outlet tubes, are obvious, and all such obvious embodiments are implied by this exemplar illustrated.

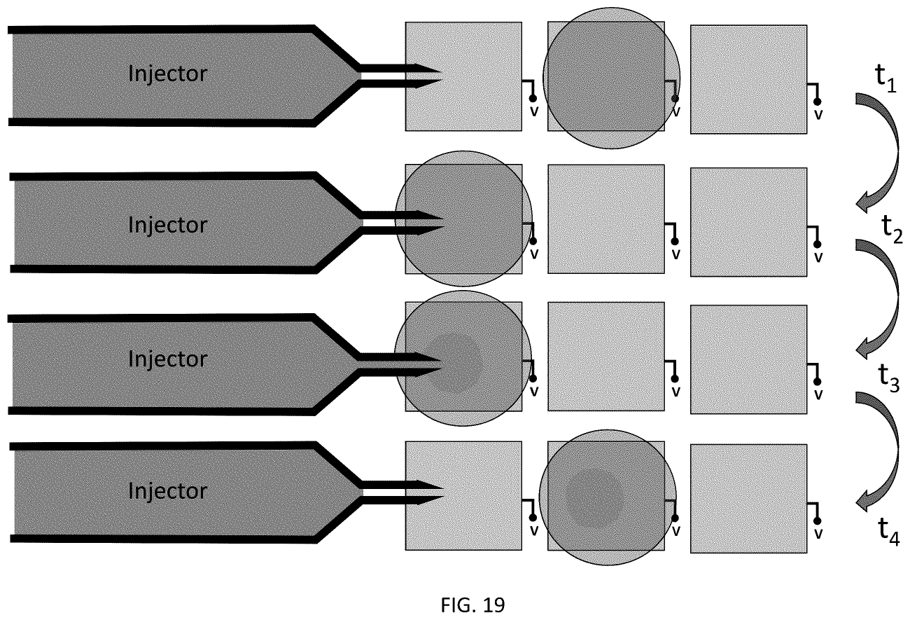

FIG. 19 shows a preferred embodiment and method for injecting a second solution into a droplet from the electrode array. At the initial time t₁, shown at top, an injector tube, having a fine, sharp tip much smaller than the droplets, and loaded with a second solution, is positioned near to the electrode array, and a droplet is moved to a location nearby to the tip. At time t₂, shown below, the adjacent electrode to the tip is activated, moving the droplet to be pierced by the injector tube tip, as shown. At time t₃, shown below, pressure is applied to the injector tube to dispense a volume of the second solution into the droplet. At final time t₄, the array is activated to move the droplet away from the injector tube. From this disclosure, many such similar means of injection solution into droplets, with diverse geometries, including allowing parallel injectors, are obvious, and all such obvious embodiments are implied by this exemplar illustrated.

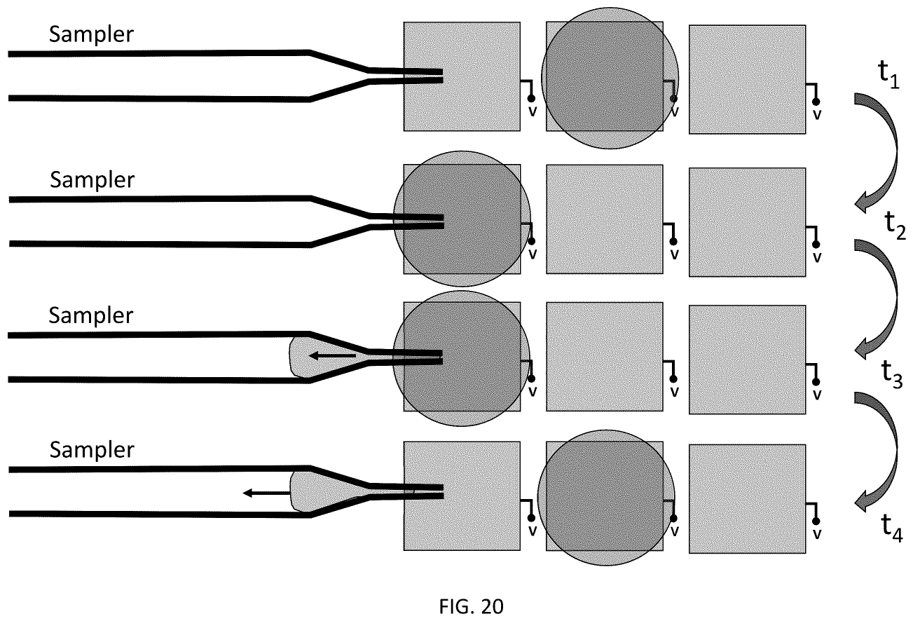

FIG. 20 shows a preferred embodiment and method for extracting a sample of solution from a droplet from the electrode array. At the initial time t₁, shown at top, a sampler tube, having a fine, sharp tip much smaller than the droplets, is positioned near to the electrode array, and a droplet is moved to a location nearby to the tip. At time t₂, shown below, the adjacent electrode to the tip is activated, moving the droplet to be pierced by the sampler tube tip, as shown. At time t₃, shown below, suction is applied to the sampler tube to extract a volume of solution from the droplet. At final time t₄, the array is activated to move the droplet away from the sampler tube. From this disclosure, many such similar means of sampling solution from droplets, with diverse geometries, including allowing parallel samplers, are obvious, and all such obvious embodiments are implied by this exemplar illustrated.

FIG. 21 shows a preferred embodiment and method for mixing the solution within a droplet on the array. As shown, starting from a single droplet, if voltage is applied at adjacent electrodes, at right, the droplet begins to elongate onto the adjacent electrodes. Then, the voltage is switched to the configuration at left, drawing the droplet back to the central electrode. Cycling this process achieves a mixing of the solution inside the droplet, with more cycles providing more mixing, to whatever extent is desired. From this disclosure, many such similar means of mixing droplets from droplets by cyclical distortions of the droplet shape, or by repeatedly dividing and re-merging the droplet, are obvious, and all such obvious embodiments are implied by this exemplar illustrated.

### Cell Packaging of DNA Payloads and Subsequent Processing

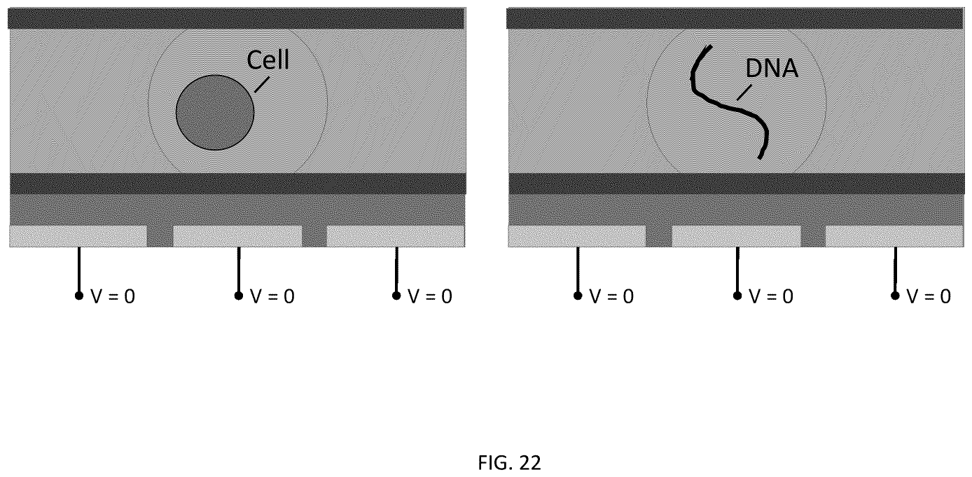

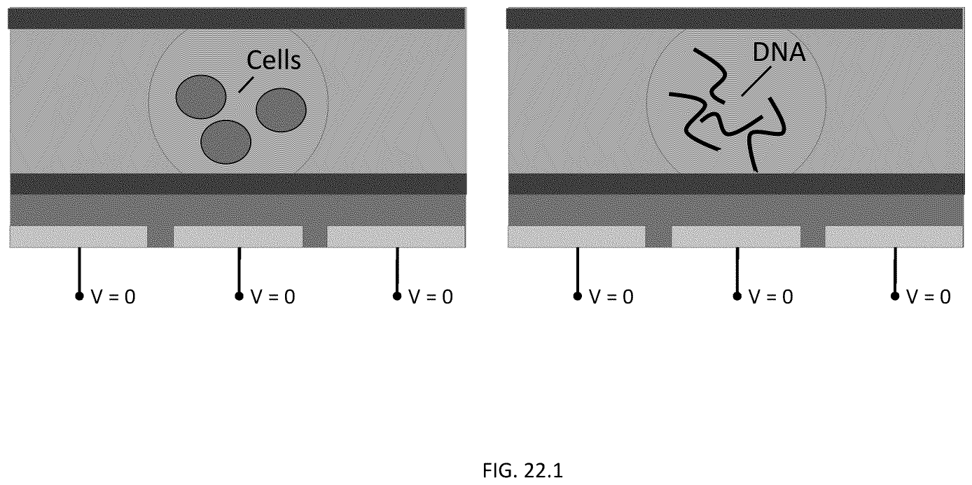

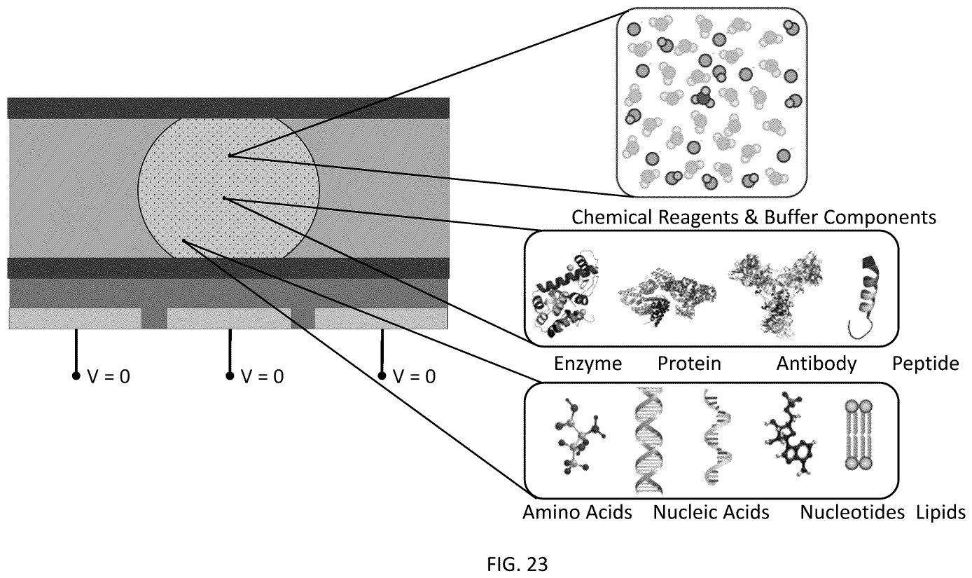

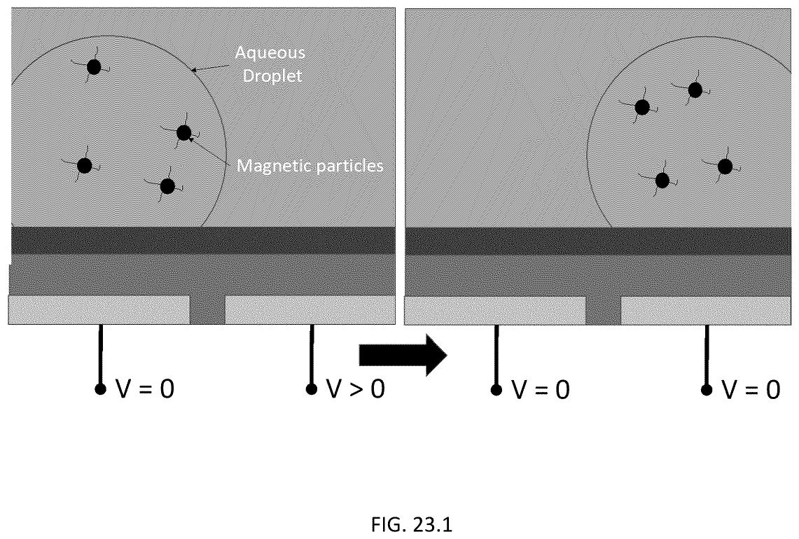

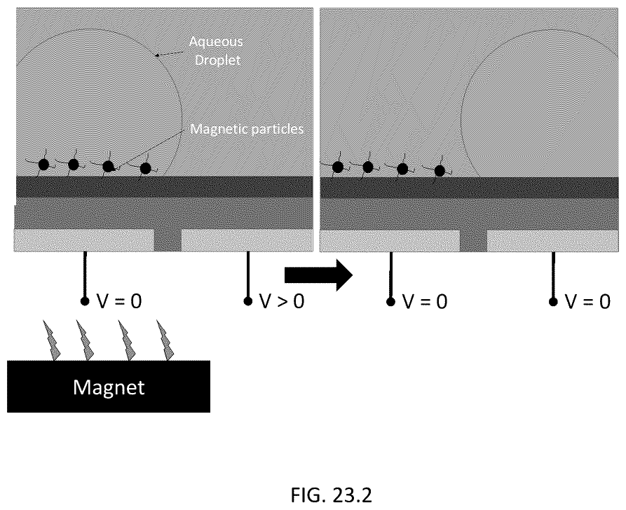

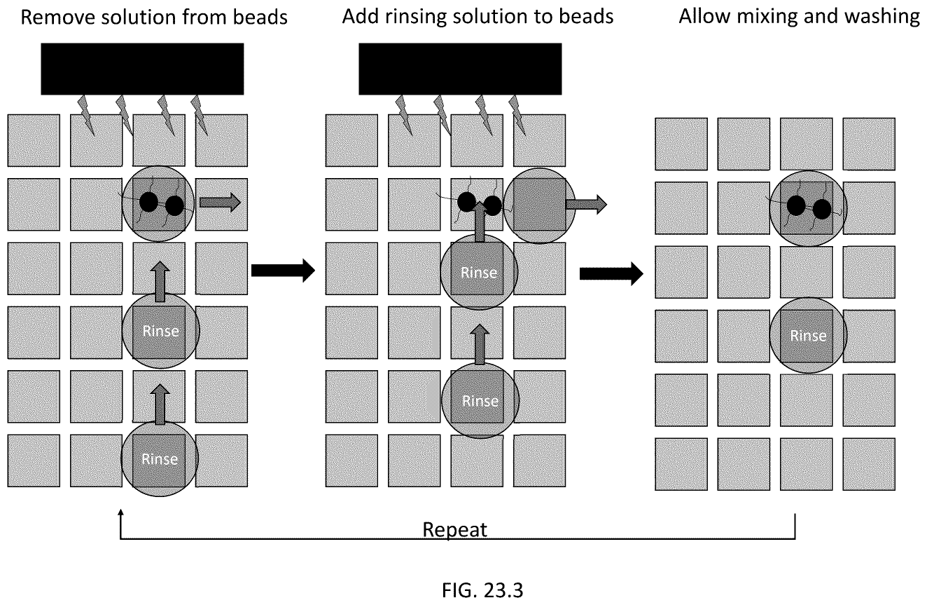

It is an object of this disclosure that the droplets on the EWOD array can be used to deliver DNA packages to cells, for the purposes of synthetic biology, and specifically to engineer the genome of said cells. Towards this end, FIG. 22 shows compositions of droplets on an electrowetting array that carry a cell (left), or a DNA payload molecule (right). As shown in FIG. 23, droplets could also carry a multiplicity of cells (left), or multiplicity of DNA payload molecules (right). In preferred embodiments, as indicated, such droplets may carry cellular material, or DNA payload material, across the array, delivering them to desired sites on the array. FIG. 23 shows that such droplets on the array could also carry diverse types of biomolecules or biochemical reagents. Such constituents could include salts, ions, and buffer reagents, as well as biomolecules such as proteins, enzymes, lipids, antibodies, amino acids and peptide, nucleic acids and oligomers, nucleotides, and others such biomolecules as occur within cells and biological organisms. It is also well known that such biomaterials, in contact with solid surface, can stick to or “foul” such a surface, such as for example proteins adhering to a surface, or cells adhering to a surface. In the context of EWOD devices in which the hydrophobic or dielectric surfaces may be exposed to such biomaterials, it is in preferred embodiments desirable to avoid such fouling, which can degrade device performance. For this purpose, such exposed surfaces can be coated with anti-fouling materials, or in preferred embodiments, nano-patterning of the surface itself can prevent such fouling, including cell adhesion, without the need to introduce additional chemical exposures and materials compatibility constraints in the system. Conversely, in certain embodiments where cell adhesion may be desirable, such as growing adherent cultures or organoids in certain zones or over certain electrodes of the EWOD device, the surfaces can be chemically functionalized or, in preferred embodiments, nano-patterned to promote cell adhesion and growth.

In some embodiments, the payloads carried by the droplet may include magnetic beads or more generally particles. Many forms of magnetic particles are commercially available and may be derivatized in numerous ways to generate affinity reagents to capture a desired molecular species from solution. In the absence of a magnetic field, the particles may co-migrate with a droplet on the EWOD array, as shown in FIG. 23.1. While in the presence of a sufficient magnetic field, the particles may be held in place upon the array as the droplet is transitioned to another site, as depicted in FIG. 23.2, effectively separating the beads from solution while retaining any tightly bound compounds so that they are separated from their droplet of origin. This approach may be used in workflows for performing forms of molecular analysis not easily amenable to on-chip sensing operations, as well as steps of purifying or concentrating the bound species.

Depending upon the nature of the beads, sample of interest, desired purity, and overall system architecture, removal of beads from a droplet may cause some disruptions to surface tension which leave residual amounts of the original droplet with the population of beads. In some embodiments, the beads may be rinsed by the process depicted in FIG. 23.3, where a new droplet of rinse buffer is brought into contact with a group of beads, the magnetic field is reduced or removed allowing the beads to be resuspended in solution with optional droplet mixing steps, before reapplying the magnetic field and removing the droplet of rinse buffer. The process may be repeated as many times as needed for the application of interest. A similar process may be used for solution exchange purposes.

In certain embodiments, the magnetic field may be imposed by the integrated electronics (such as an electromagnet) in the EWOD device, while in other embodiments the magnetic field may be imposed by proximity to an external magnet that may be above, below, or located laterally to the EWOD array. The lateral embodiment may be used to enable in droplet bead separation, as has been illustrated in reference “In-Droplet Magnetic Beads Concentration and Separation for Digital Microfluidics,” by Y. Wang, Yuejun Zhao, and S. Cho, able to be accessed at:

https://www.semanticscholar.org/paper/In-Droplet-Magnetic-Beads-Concentration-and-for-Wang-Zhao/57cba6de3c5f406b52b9c584ffc7bde34c0b86a9.

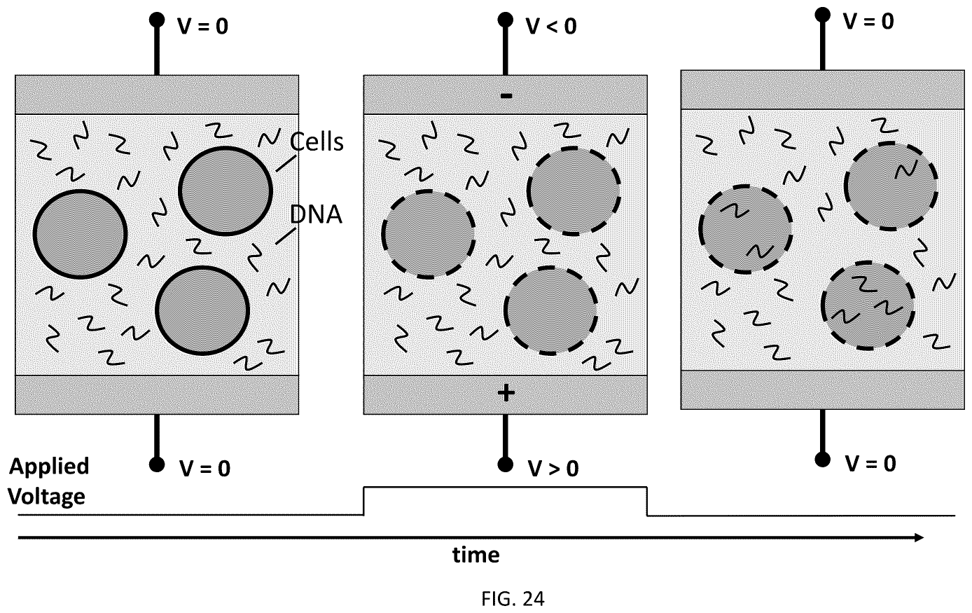

FIG. 24 illustrates the well-known process of electroporation. In this process, a suspension of cells and DNA molecules (or other payload molecules intended for delivery into the cells) is placed between electrodes, and a voltage pulse or pulses are applied to the solution using the electrodes, as shown in the voltage timing diagram, which causes the temporary formation of pores in the cell walls. Subsequent to pore formation, the DNA molecules can enter the cell, and the pores eventually seal up, with the net result that DNA (or other payload molecules) has been delivered into the cell. This is a common and well-known means of introducing DNA, or other molecules, into cells.

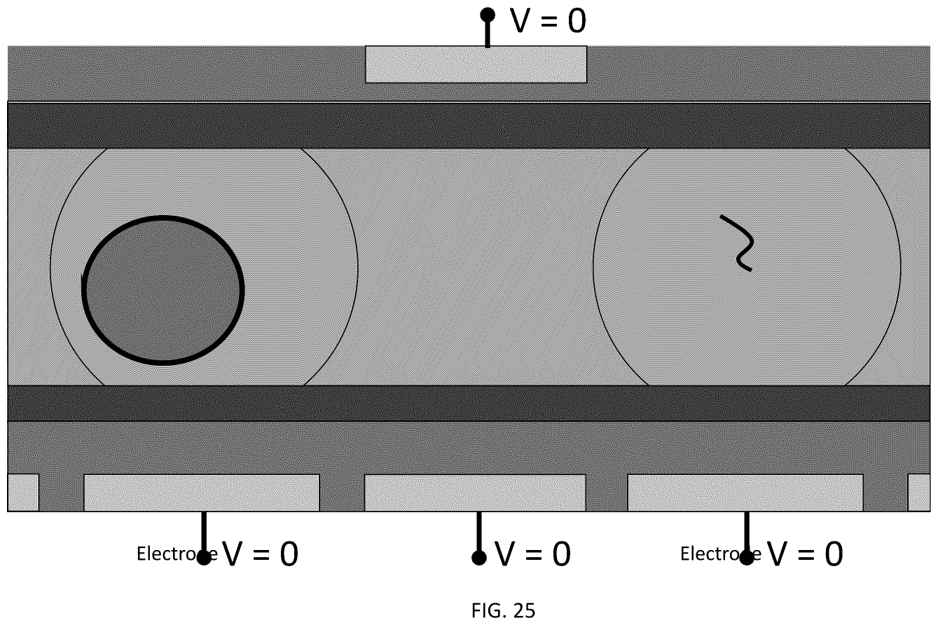

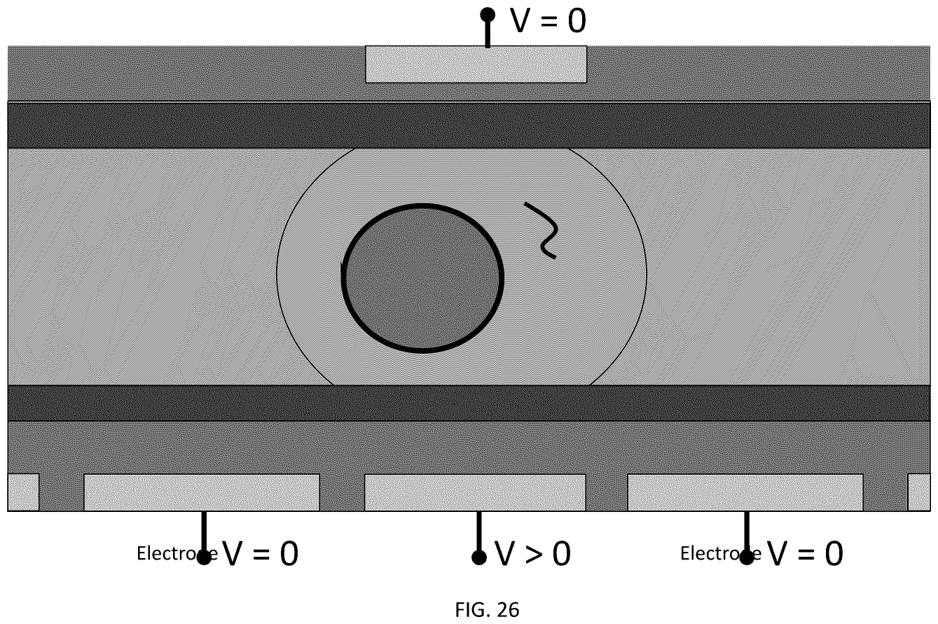

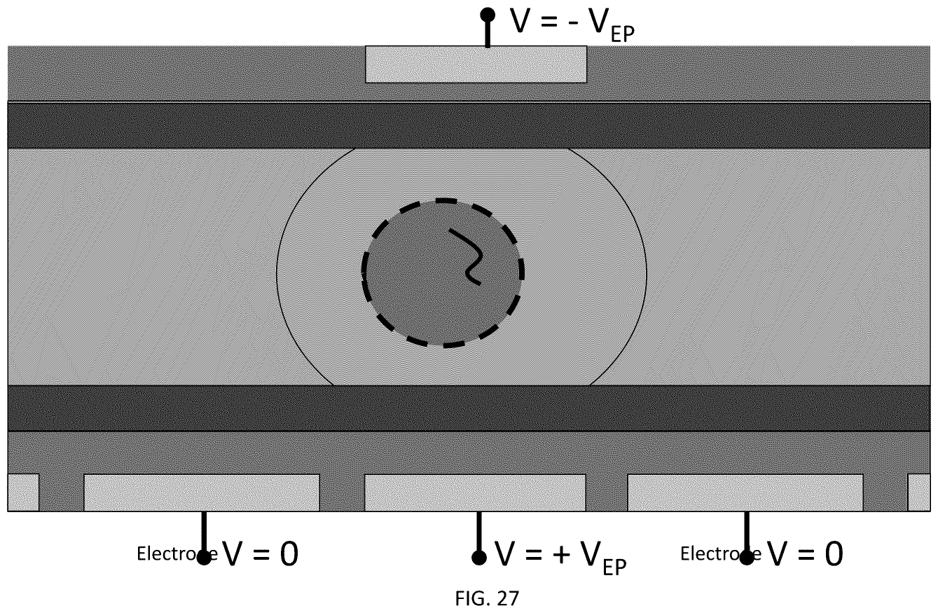

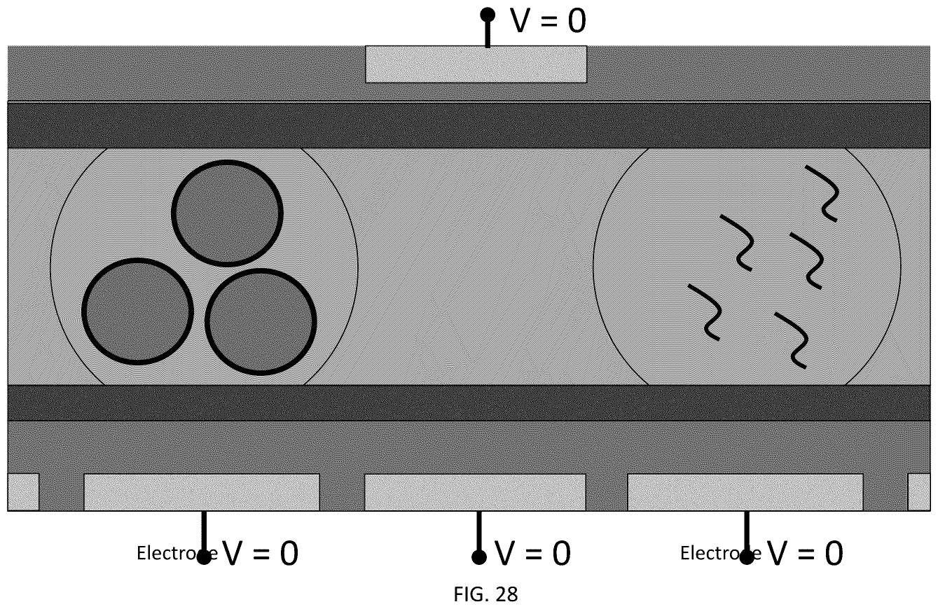

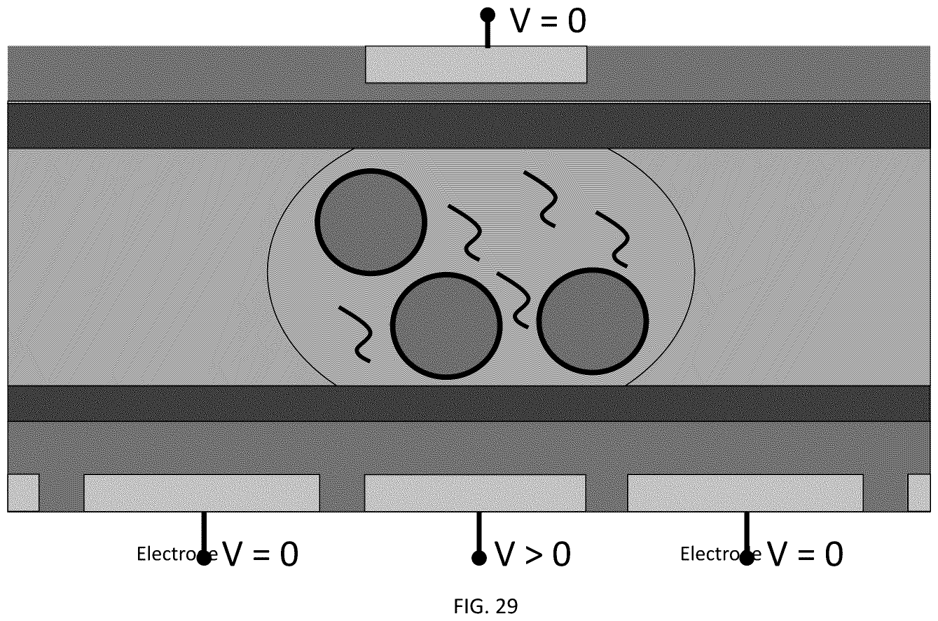

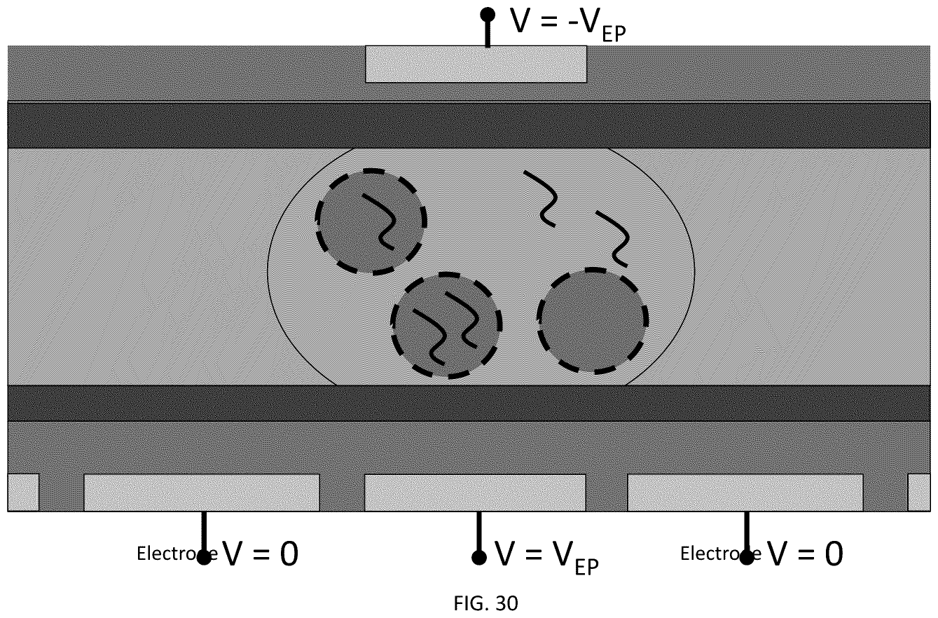

FIGS. 25, 26, and 27 show embodiments and methods for delivering DNA “payload” molecules into cell “packages” on an EWOD device via the process of electroporation, effecting the delivery of the payload into the cell for applications such as making engineered cells for synthetic biology. FIG. 25 shows the starting configuration, in which a droplet containing a cellular package and a droplet containing a DNA payload are positioned near each other on the array. Next, as shown in FIG. 26, the central electrode is activated, causing the droplets to merge onto that electrode, resulting in the cell and DNA residing in the same droplet. Finally, as shown in FIG. 27, the upper and lower electrodes shown (which may include use of the lower electrowetting electrode as shown, or may use auxiliary electrodes not shown) are pulsed with voltages (indicated as equal and opposite, +V_{EP} and −V_{EP}, although unequal voltages could also be used) that induce the formation of pores in the cell as per electroporation, and resulting in the DNA payload entering the cell. FIGS. 28, 29, and 30 illustrate the similar process, but with a multiplicity of cell packages and DNA payloads in the droplets, to indicate that this process may be done at the single cell/single molecule level, or also at multi-cell/multi-molecule. The multi version of this may have the advantage of improving the yield of properly loaded cells, owing to the large number of loading opportunities.

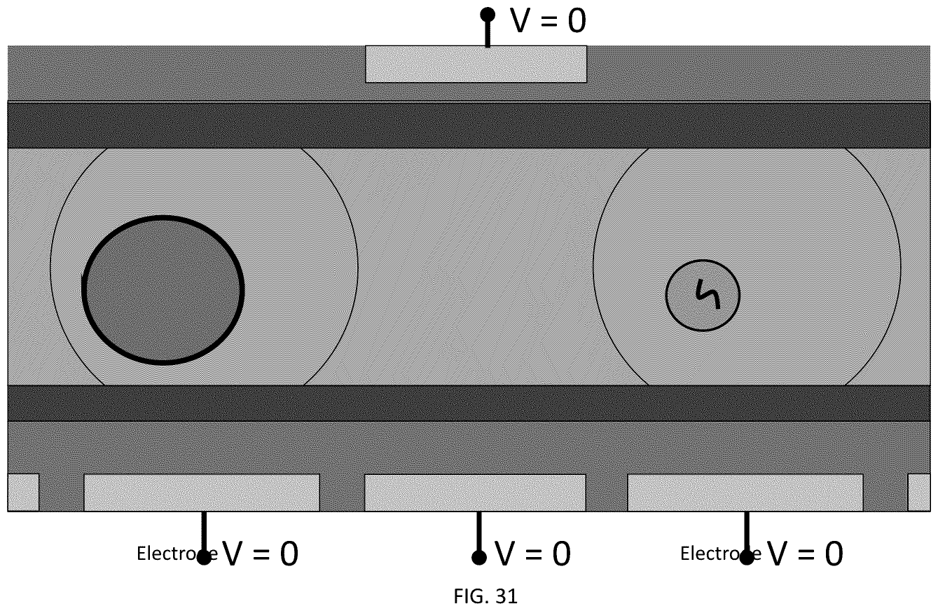

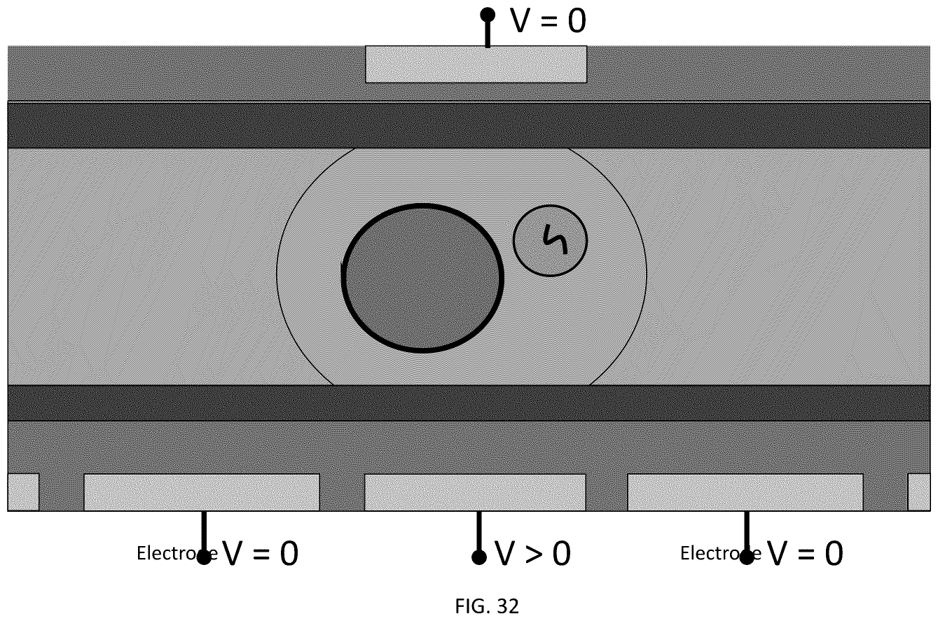

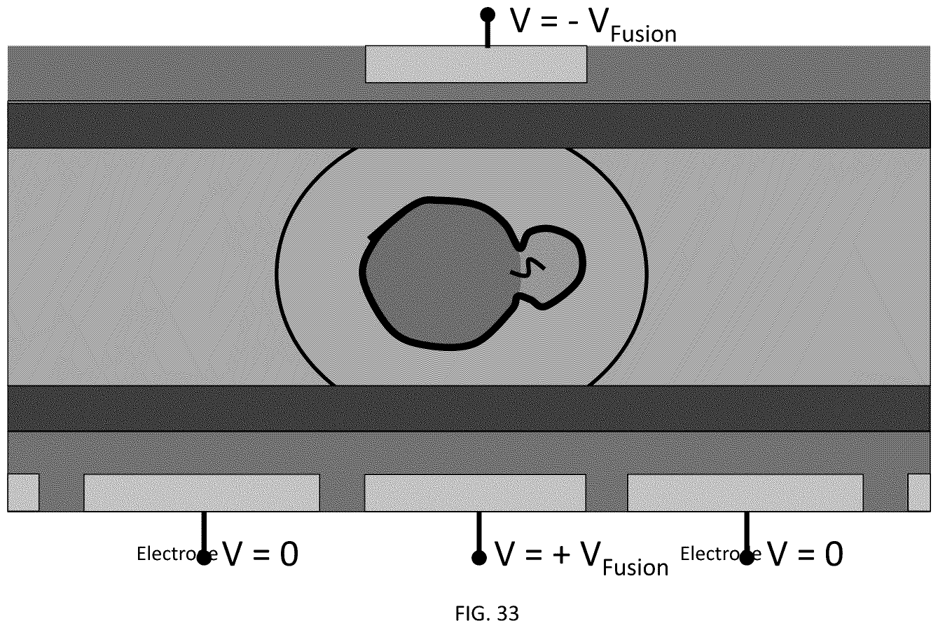

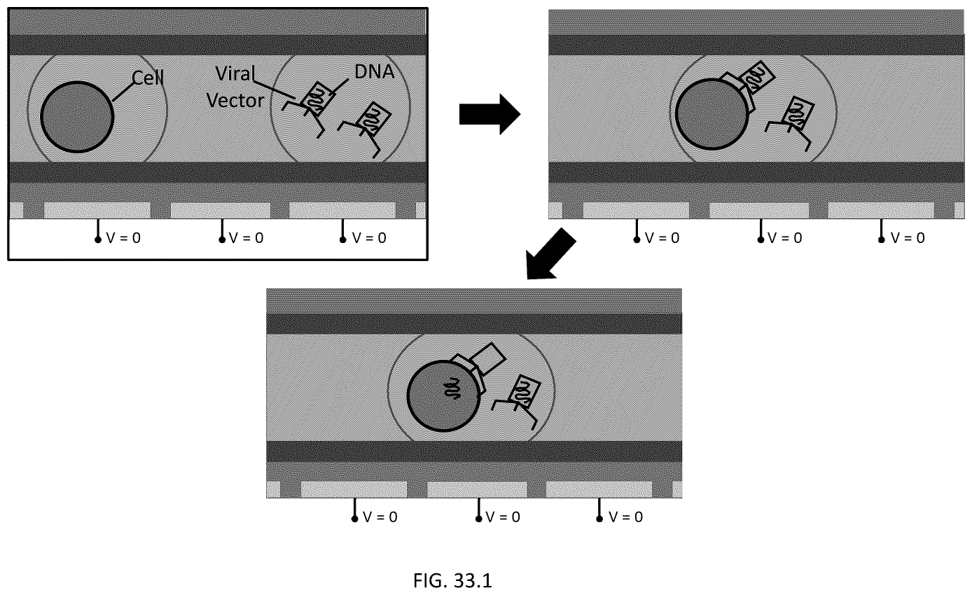

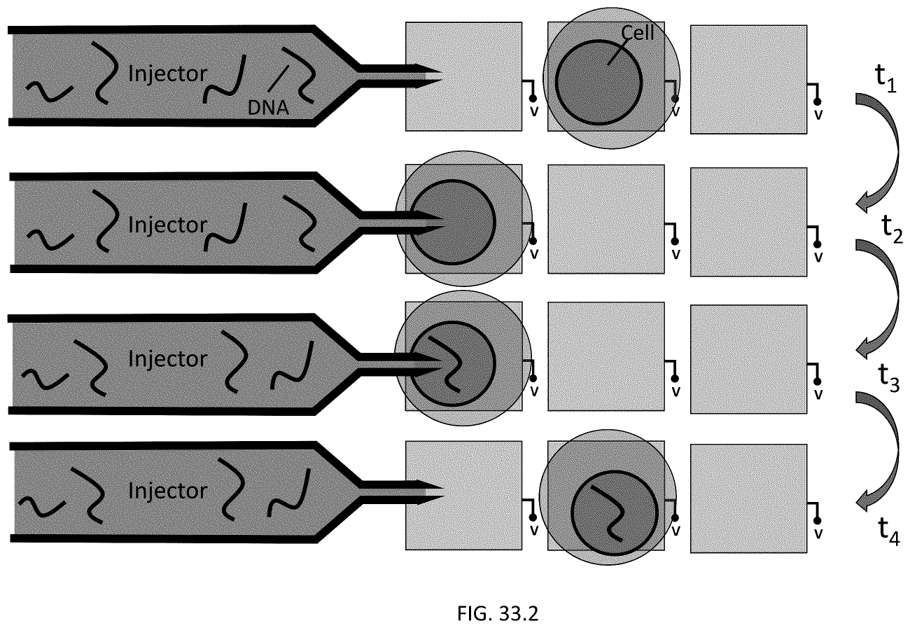

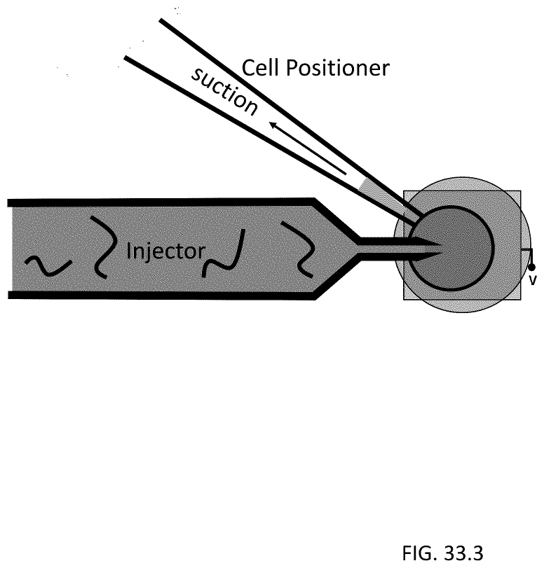

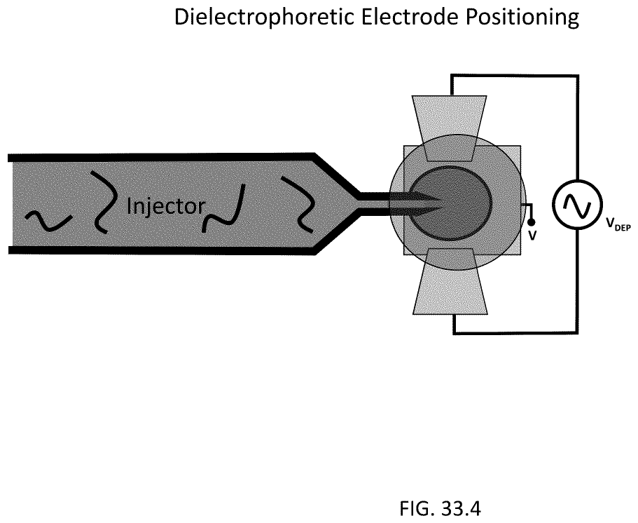

FIGS. 31, 32, and 33 show embodiments and methods for delivering DNA “payload” molecules into cell “packages” on an EWOD device via the process of vesicle fusion, effecting the delivery of the payload into the cell for applications such as making engineered cells for synthetic biology. FIG. 31 shows the starting configuration, in which a droplet containing a cellular package and a droplet containing a vesicle containing DNA payload are positioned near each other on the array. The vesicle here may be a lipid vesicle, for example. Next, as shown in FIG. 32, the central electrode is activated, causing the droplets to merge onto that electrode, resulting in the cell and delivery vesicle residing in the same droplet. Finally, as shown in FIG. 33, means are applied to promote the vesicle fusion with the cell to achieve the payload delivery into the cell. Such means may include upper and lower electrodes shown (which may include use of the lower electrowetting electrode as shown, or may use auxiliary electrodes not shown) being pulsed with voltages (indicated as equal and opposite, +V_{Fusion} and −V_{Fusion}, although unequal voltages could also be used) that promote the vesicle fusion, and or other means of promoting fusion may be applied, such as having chemical factors in the droplet that promote fusion, or delivering additional fusion promoting reagents, either by merging with additional reagent delivery droplets, or using an injector to inject such reagents, and resulting in the DNA payload entering the cell. While a single cell, single vesicle embodiment is shown, multiple cells and multiple vesicles could also be used, to promote higher yield of properly loaded cells.

From these disclosures of methods using electroporation or vesicle fusion to effect delivery of the DNA payload to the cell package in the EWOD droplets, it would be obvious to one skilled in the arts of synthetic biology and cellular engineering that many variations on these methods, or comparable methods of payload delivery used in bioengineering, can be employed in this setting. There are many such known methods of transfection or transduction, such as fusion with an exosome or bacteria cell or yeast cell to deliver a payload, or infection of the cell with a virus carrying the payload to deliver a DNA payload via transduction, or diverse other methods of transfection to deliver DNA payloads through cell walls, or also the delivery of RNA payloads. All such obvious variations and alternatives for EWOD-controlled payload delivery, based on these and other such known methods of payload delivery are meant to be implied by the specific embodiments using electroporation or vesicle fusion shown here. Such methods include sonication or sonoporation. In particular, recent developments in sonoporation have seen the monolithic integration of aluminum nitride, piezoelectric ultrasonic transducers on CMOS wafers, which achieved low power consumption (3 mW), low bias voltages (>2 V), and high frequencies (>1 GHz). Such transducers can be integrated into EWOD devices to drive payload delivery.

FIG. 33.1 shows the use of a viral vector to perform transduction to deliver a DNA payload to a cell. As shown in the time series of panels, the cell and viral vectors are delivered in separate droplets. The droplets are merged, at which point the viral vectors can attach to the cell and inject or otherwise deliver the DNA payload through the action of the virus on the cell wall, such as by endocytosis or other viral processes.

In the diverse electroporation methods shown in FIGS. 24 through 33.1, the efficiency of payload delivery might depend on the ratio of droplet volume to cellular volume, and in preferred embodiments, the droplet volume may be reduced to increase concentrations of the reactants and thereby drive more rapid or efficient payload delivery. In preferred embodiments, such volume reduction may be accomplished by splitting off a portion of the droplet solution using electrowetting motions, or absorbing a portion of the droplet solution into a porous media brought into contact with the droplet, or by allowing or driving the droplet to partially evaporate, or by merging into the droplet another droplet that contains volume exclusion or crowding reagents (such as Dextran or PEG or many others obvious to those skilled in biochemistry), or absorptive reagents that may absorb and sequester a portion of the solution in the droplet, effectively increasing concentration of the payload and cells and other payload delivery reagents in the droplet.

FIG. 33.2 shows the use of an injector to directly micro-inject DNA payloads into a cell. The injector contains DNA payloads. The target cell is encapsulated in a droplet and is transported to the site adjacent to the injector, such that the injector pierces the cell wall. A DNA payload is then micro-injected into the cell interior, and the cell is subsequently removed back to the array for further processing.

FIG. 33.3 shows a preferred embodiment of direct injection, in which an auxiliary hollow tip with a fixed position, applies suction to the droplet, and attaches to the cell, thereby locating and holding the cell in a fixed, known position for precision injection into the cell. This provides the ability to place the cell into a known location and increase the yield of successful cell injections. From this disclosure, it is obvious to one skilled in micromanipulation that many other variations and options for mechanical cell positioning are possible, which would serve to bring the cell in the droplet to a defined location for precise injection. Such options would include mechanical structures that trap the cell in a defined location. It is obvious from this disclosure that many mechanical structures can be used to trap and hold cells in this way, and all of these obvious variations are meant to be covered by the present example.

In other preferred embodiments it may be desirable to have non-mechanical means for such precision cell location control, where a cell can be moved specifically through solution, or be held in place at a specific location in a solution using electrical forces. It is well known to those skilled in cell biology that the dielectrophoretic force is an electrical force that can be used to precisely and selectively move or hold a cell within a fluid volume, and without bulk motion of the fluid. In preferred embodiments this electrical force is applied to a cell through a pair of auxiliary electrodes, which attract the cell to the gap between electrodes whenever a suitable driving AC voltage is applied to the electrodes. FIG. 33.4 depicts another preferred embodiment in which a dielectrophoretic probe stabilizes the cell within the droplet. The means and conditions for doing this are well known to those skilled in dielectrophoresis. This dielectrophoretic forcing mechanism can be applied independently of the EWOD driving forces, for example a droplet may be help in place by EWOD forces and electrodes, while dielectrophoresis electrodes within the droplet draw a cell in the droplet to that precise location, such that the cell moves through the stationary droplet, to the located directed by the dielectrophoresis electrodes. In preferred embodiments this can be used to position and hold cells for payload injection, or for payload delivery through electroporation or chemoporation.

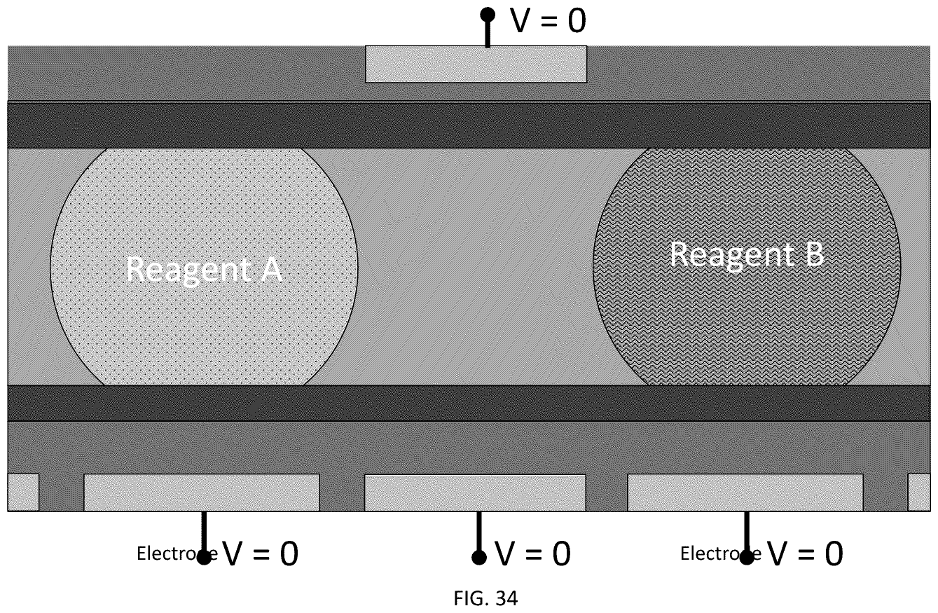

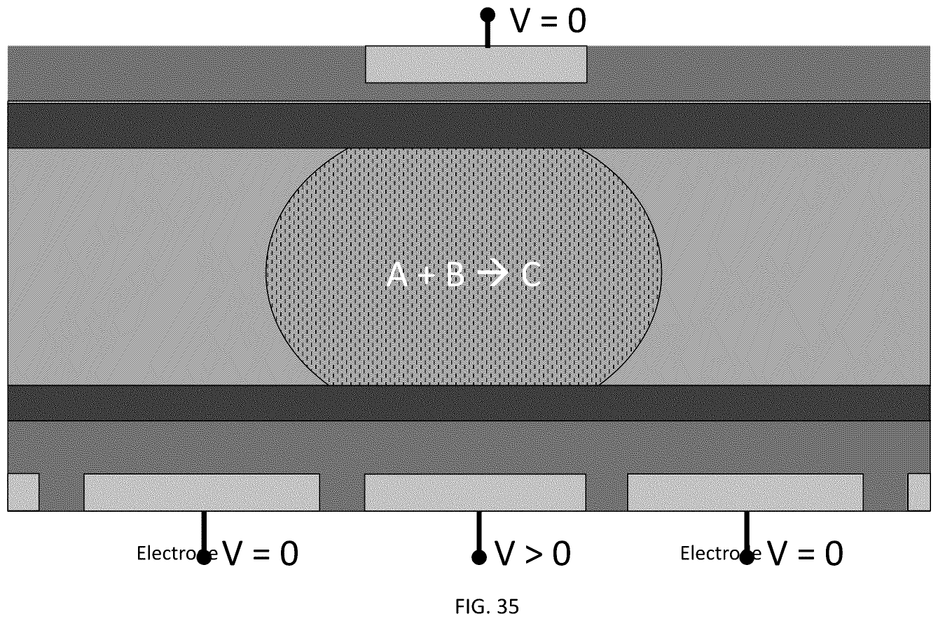

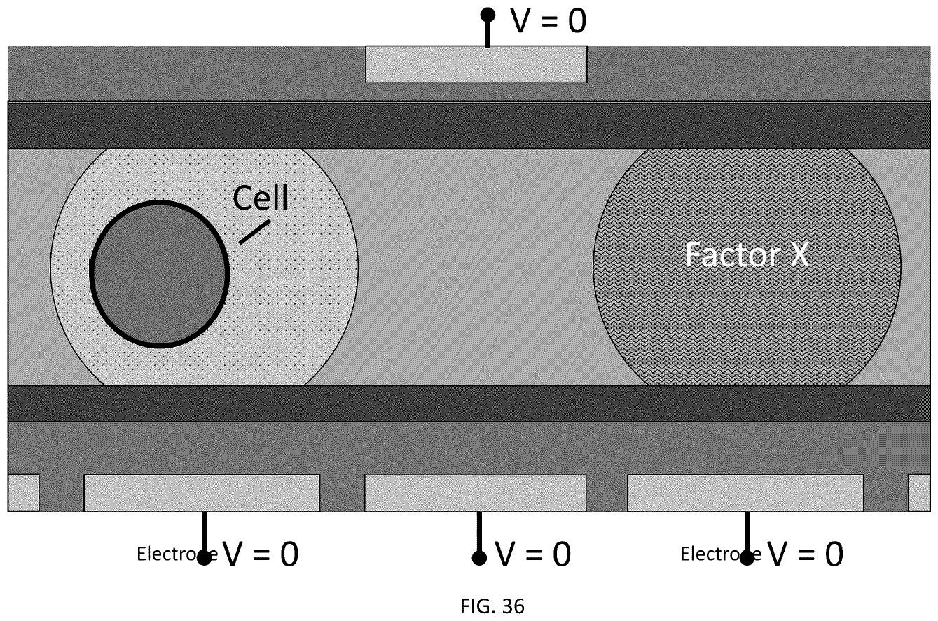

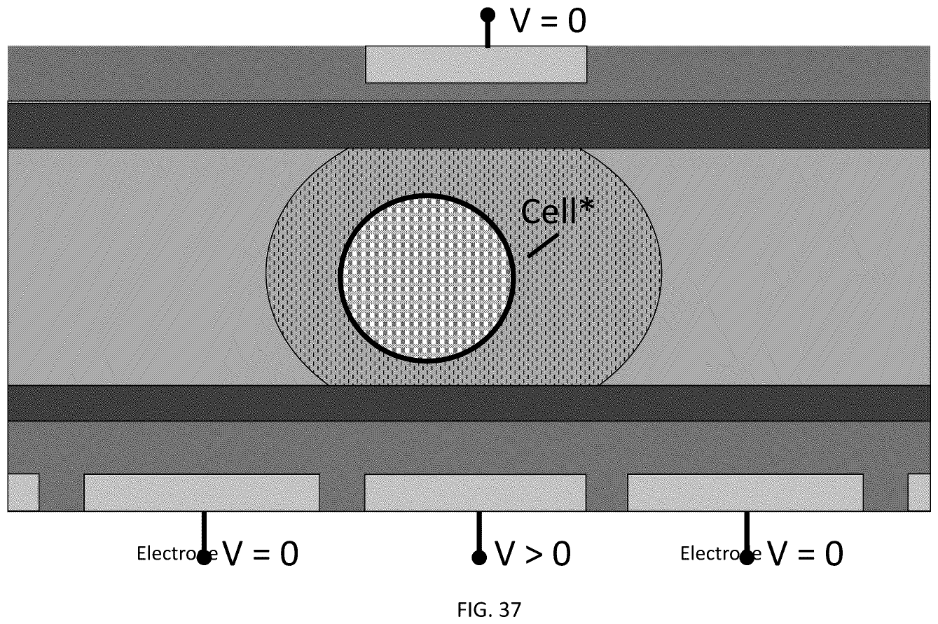

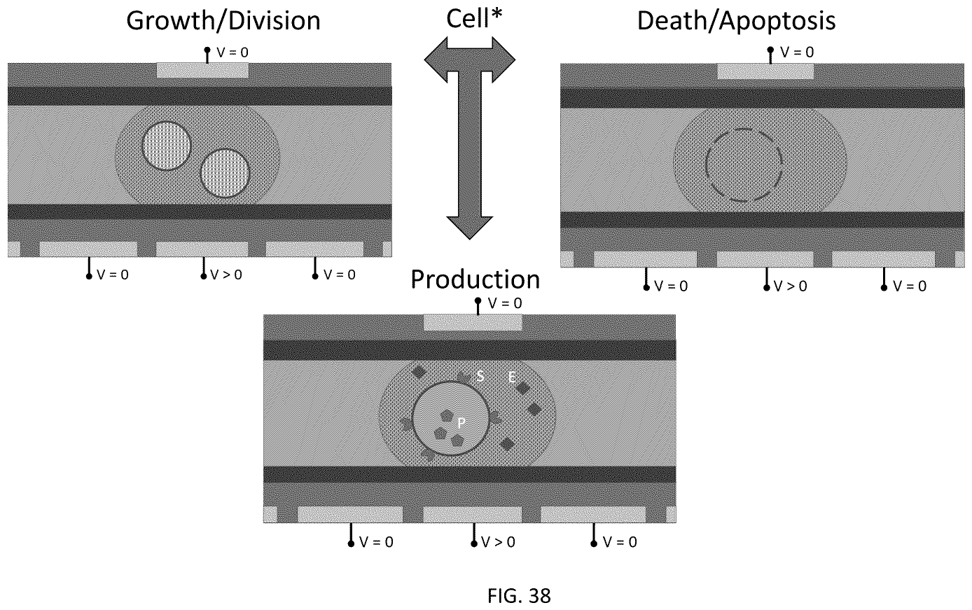

It is an object of this disclosure that the EWOD array can be used to perform assays on cells, such as the cells that have had a DNA payload delivered into them. Towards this end, as shown in FIG. 34, for the purposes of performing a general chemical reaction of the form A+B→C, with reactants A and B, and product C, this can be performed on the EWOD array by having one droplet containing reactant A and one containing B, and then, as shown in FIG. 35, the droplets can be merged to drive the reaction A+B→C in the resulting droplet. In one preferred embodiment, shown in FIG. 36, one reactant is a cell, and the other is a stimulating factor X, and the reaction is cell+X→cell*, an activated form of the cell, as shown in FIG. 37. The result of such a stimulation, or a series or combinations of such stimulations of the cell by multiple factors, in parallel or in series. The result of such stimulation could be, for example, as indicated in FIG. 38, inducing cell division or cell growth (left), inducing the death, or apoptosis, or other disruption of the cell (right), or (center) possibly activation of biochemical synthesis pathways resulting in various outcomes, such as the production of a chemical product, P, within the cell, or the production of cell surface receptors or markers or other molecules, S, or the excretion outside the cell of excreted molecules or factors, E. These outcomes are then subject to detection by sensor circuits on the array, as further disclosed below, for the purpose of recording an outcome of the cell stimulation in question, such as in the greater context of discovering which DNA payloads deliver the desired functionality to the cell.

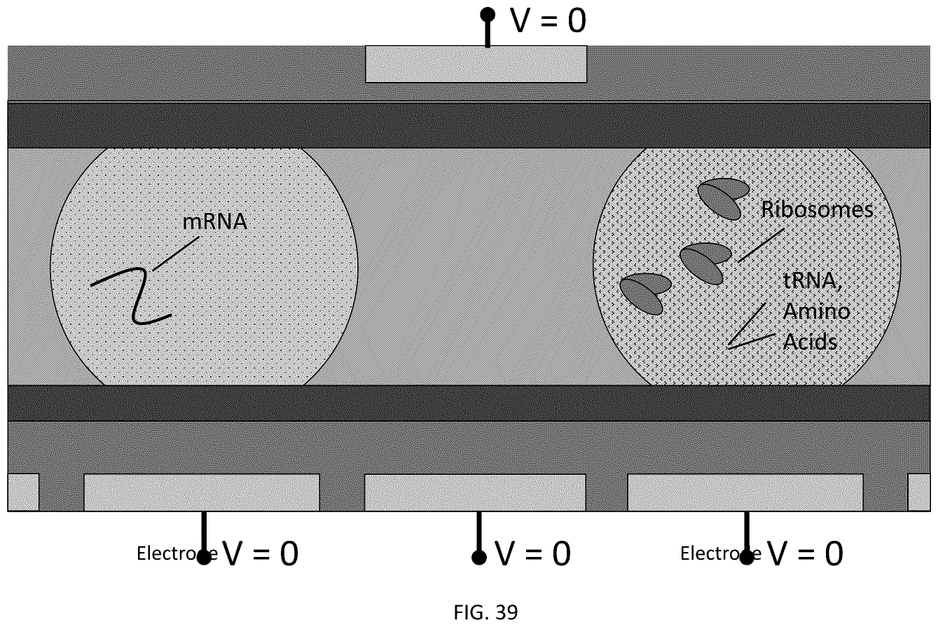

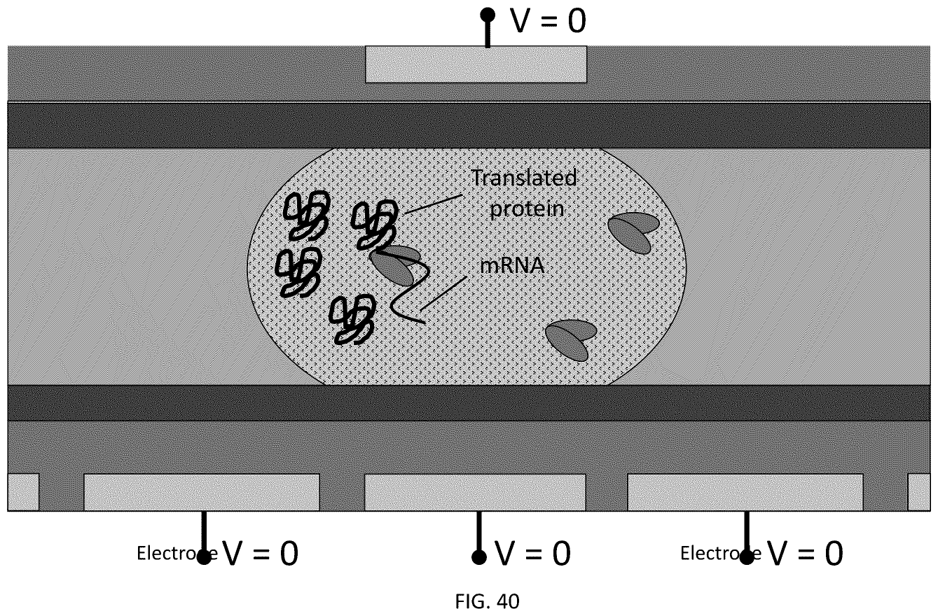

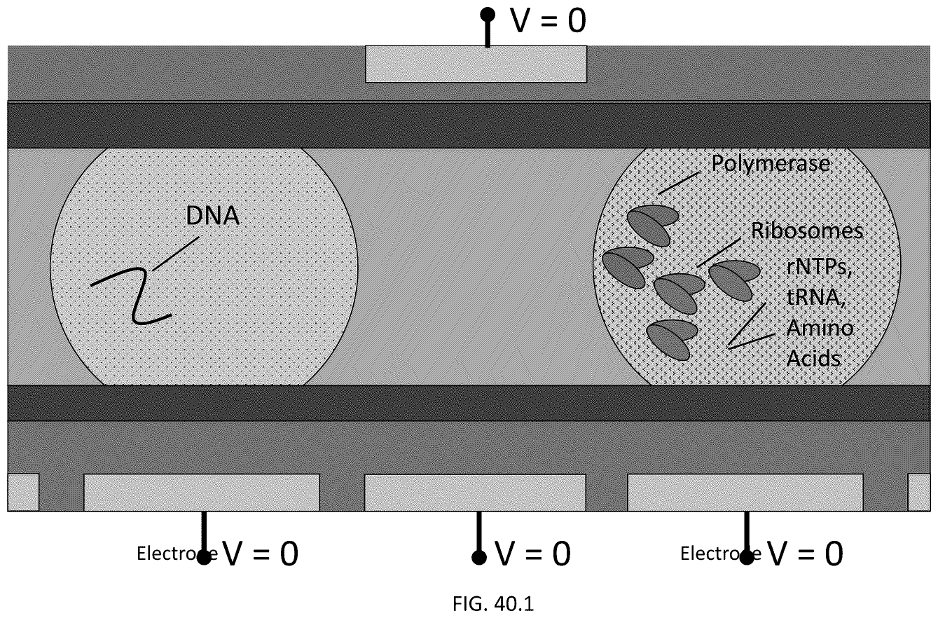

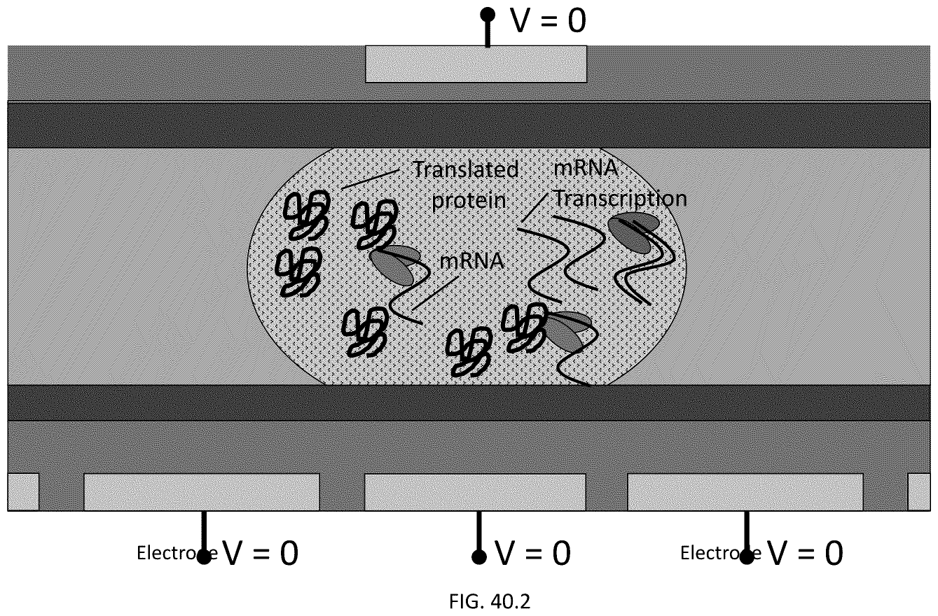

One preferred embodiment of this general disclosure of EWOD driven cell packaging and resulting functionality is the special case that makes use of cell free protein expression technique, as illustrated in FIGS. 39 and 40. Cell free protein expression is a well-known process, wherein mRNA translation and protein expression are performed in a test tube, without the use of a biological cell, by simply reacting mRNA with the ribosome, amino acids, transfer RNA, and other biochemical reactants required to perform translation of the mRNA into protein. In the present setting, it is an object of this disclosure that this can be done on the EWOD array, in the manner above, in a case where a droplet itself plays the role of the “cell container”, and the delivery of the DNA payload is here an mRNA payload. As shown in FIG. 39, there is initially provided a droplet containing the mRNA payload, and another—playing the role of the cell package—containing the ribosomes and other reactants, and upon merging the droplets as in FIG. 40, delivering the mRNA payload, the translation reaction is activated, resulting in functional expression of the protein of interest. In another preferred embodiment, shown in FIG. 40.1 and 40.2, the payload could be a DNA payload, in which case the cell free expression reactants in the other droplet include polymerase and rNTPs, and other components as needed to transcribe DNA into mRNA, which is then translated by the ribosomes in the encoded protein. In preferred embodiments, the cell free expression may achieve the production of more than one target protein: there may be multiple different mRNA delivered in the payload, encoding for multiple different proteins, or multiple different DNA delivered in the payload, encoding for different proteins, or a single longer DNA molecule that encodes for multiple different mRNA, resulting in multiple different proteins being produced.

### Sensor Integration Into the EWOD Array

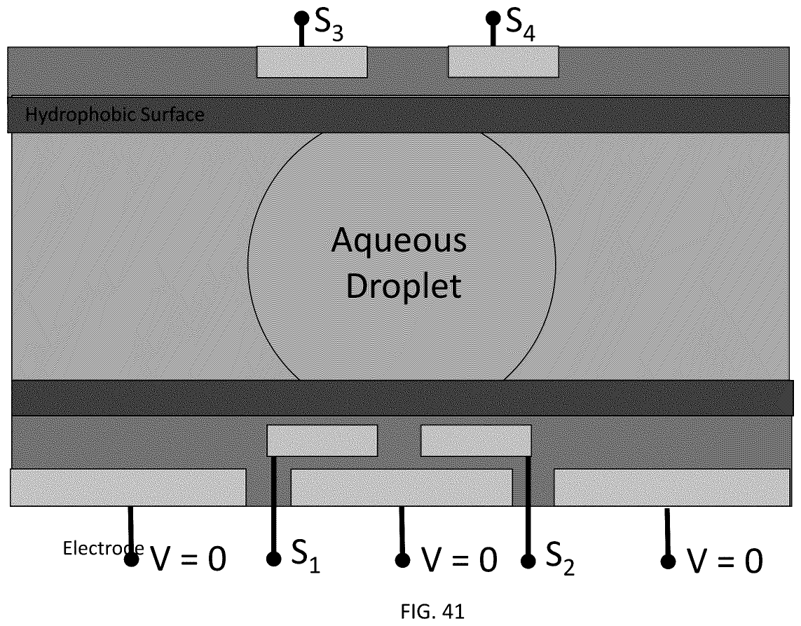

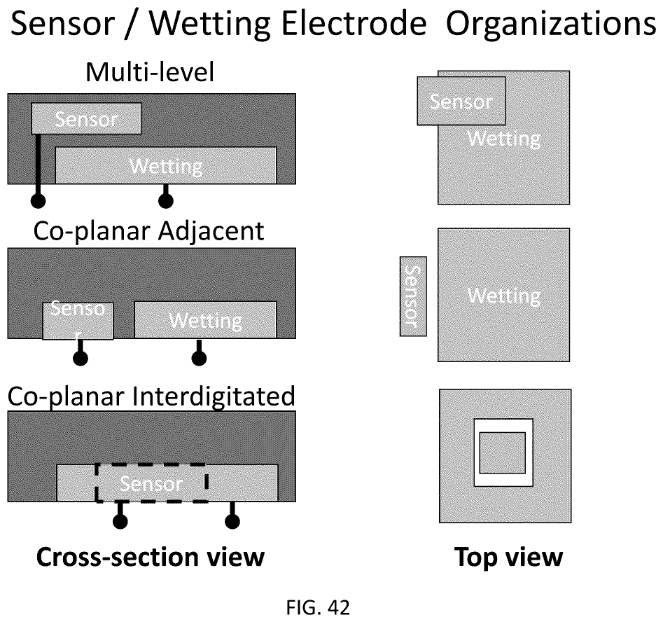

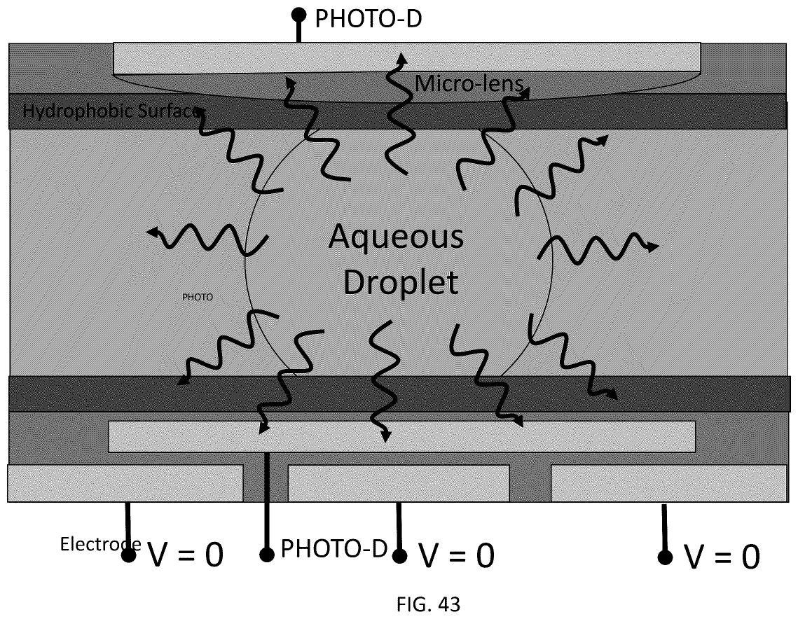

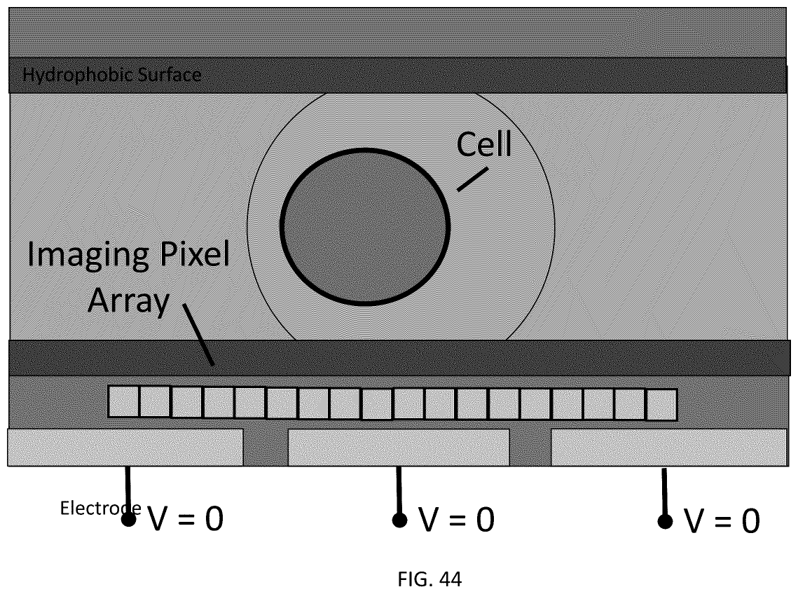

FIGS. 41 and 42 show preferred embodiments in which electronic sensor electrodes or elements may be arranged relative to the EWOD control electrodes. Depending on the type of sensor, preferred embodiments may have the electrodes embedded in the upper surface, as for sensor S₂ and S₃, or they may be interspersed with the wetting electrodes such as for sensor S₁ and S₂ (details of the sensor circuitry are not shown). The relative positioning and organization of the sensor elements in various preferred embodiments is indicated in the cross-sectional diagrams in FIG. 42. As shown in the upper panel, the sensor electrode may be at a different level than the wetting electrode, and may therefore have an area footprint, shown in the top view, that overlaps with the wetting electrode footprint. In other embodiments, shown in the middle panel, the sensor electrode can be co-planar with the wetting electrode, and sit adjacent to it, with a suitable spacing for electrical isolation. In another preferred embodiment, in the bottom panel, the sensor electrode may be co-planar with the wetting electrode, but surrounded by it, which may have the benefit of facilitating the positioning of droplets over the sensor under the control of the wetting electrode. In other preferred embodiments, indicated in FIG. 43, the sensor may be a photo-detector or photo-diode, configured to sense light that is emitted from the droplet, with the sensitive zone located on the upper surface or lower surface above the wetting electrodes, and with or without a microlens system (as indicated on the upper detector) for collecting the light emitted from the droplet. Such light may be emitted by a bioluminescent process in the droplet, or may be emitted light stimulated by an applied voltage or applied excitation illumination, such as from the excitation of a fluorescent reporter molecule in the droplet. In other preferred embodiments, as indicated in FIG. 44, the sensor may be an imaging pixel array, configured to produce an image of a certain pixel resolution of the droplet and droplet contents, such as imaging a cell within the droplet.

### Actuator Integration Into the EWOD Array

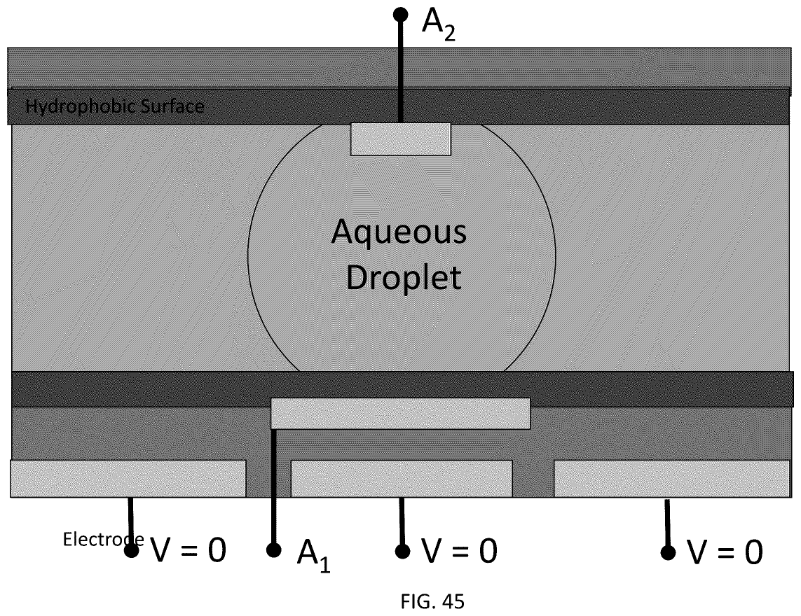

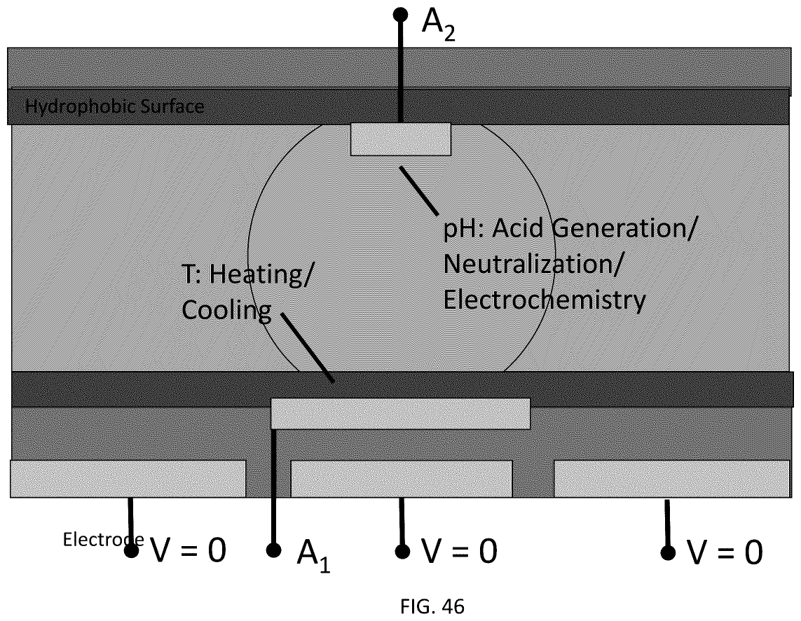

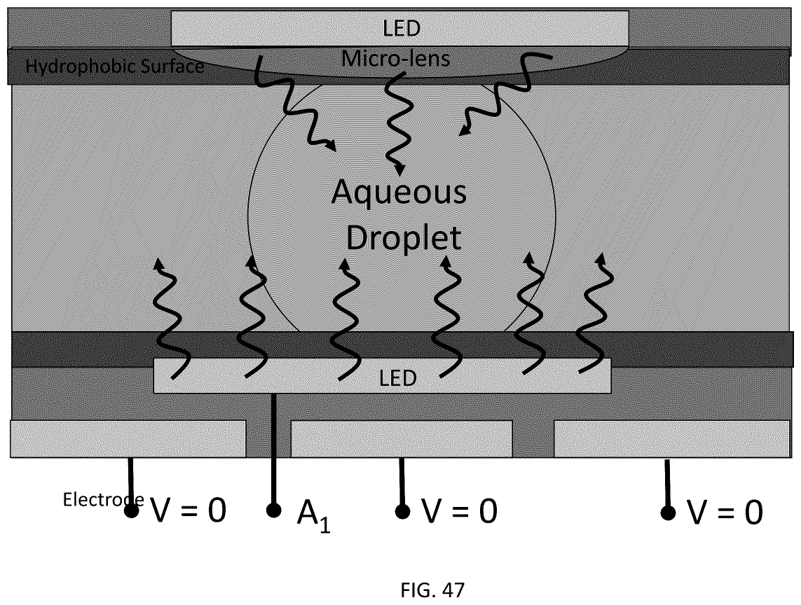

FIG. 45 shows preferred embodiments in which electronic actuator electrode or elements may be arranged relative to the EWOD control electrodes. Depending on the type of actuator, preferred embodiments may have the electrodes in contact with the droplet, as for actuator A₂, or they may be isolated from the droplet, but otherwise in close proximity, and for actuator A₁ (details of the actuator circuitry are not shown). The relative positioning and organization of the actuator elements for various preferred embodiments is indicated in FIG. 46, where for example, a temperature actuator, such as a resistive heater or a Peltier thermo-electric device, which may provide heating or cooling to the droplet, would be isolated from the droplet, but close to effect efficient heat transfer. In another preferred embodiment, an electrochemical actuator, such as which could produce acid to effect pH, or other electrochemical products, would have an electrode in contact with the droplet, as indicated in the upper sensor of FIG. 46. In other preferred embodiments, the actuator may be a light source, such as an LED, as indicated in FIG. 47, with or without a micro-lens system (indicated in the upper surface instance) for light concentration int the droplet, for the purposes of light-driven excitation or interrogation of the droplet. Such light may be a reactant for driving a chemical reaction, or may be to excite some reporter molecule, such as a fluorescent molecule,

### Parallel Operations on the EWOD Array

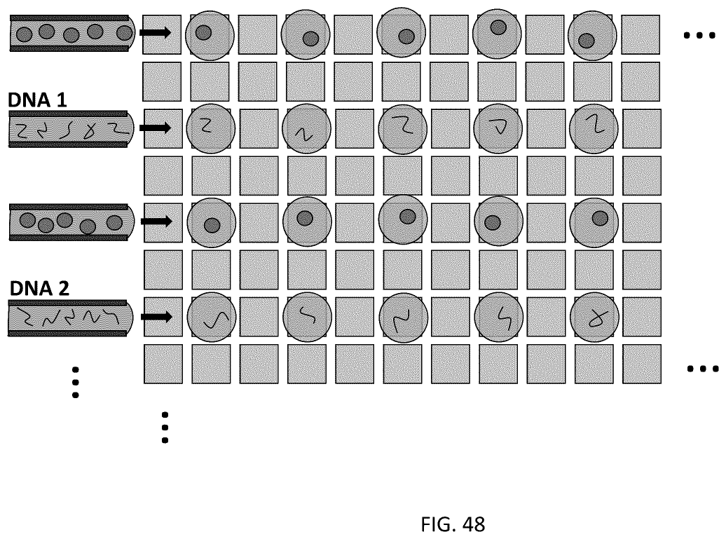

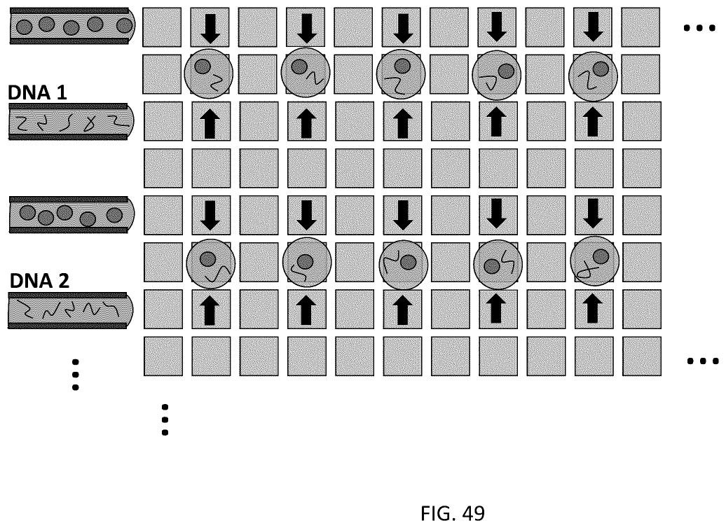

FIGS. 48, 49, and 50 show one preferred embodiment in which the EWOD array is used to perform parallel, scalable delivery of DNA payloads into cell packages. In the first step, FIG. 48 shows multiple cell droplet inlets populating the array with droplets containing cells, staged in a precise, distributed pattern on the array. There are also multiple DNA payload droplets inlets, staging droplets that contain distinct DNA payloads (types 1, 2, etc.) as determined by the different inlets, source payloads from different sources, and in a pattern on the array coordinated with that of the cellular packages, such that DNA payloads are adjacent to target cell droplets on the array. As shown in FIG. 49, the next step is to merge the respective DNA payload and cell droplets, to provide the cell and payload in the same droplets, and such that they are located over the electrodes that can provide the electroporation voltage. Then, as indicated, the electroporation is applied to drive delivery of the respective payloads into the respective cell packages. The result is an array of packaged DNA payloads, staged for subsequent manipulations.

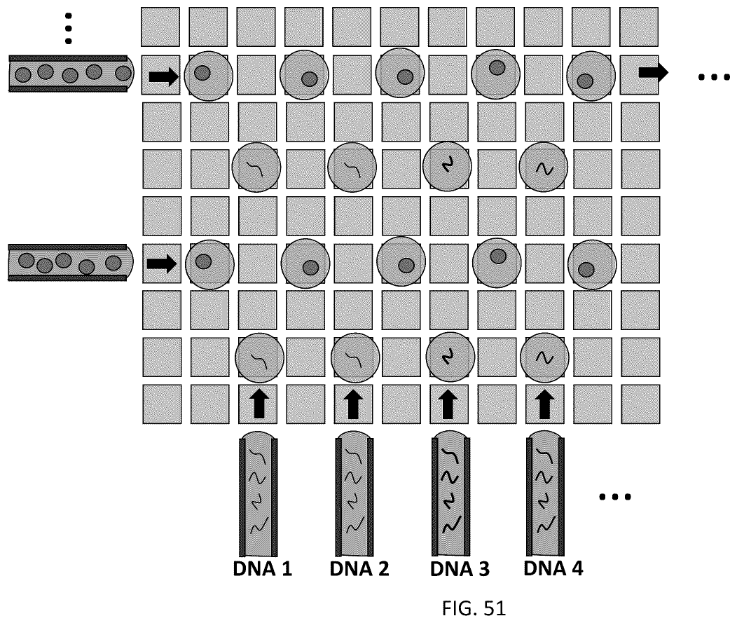

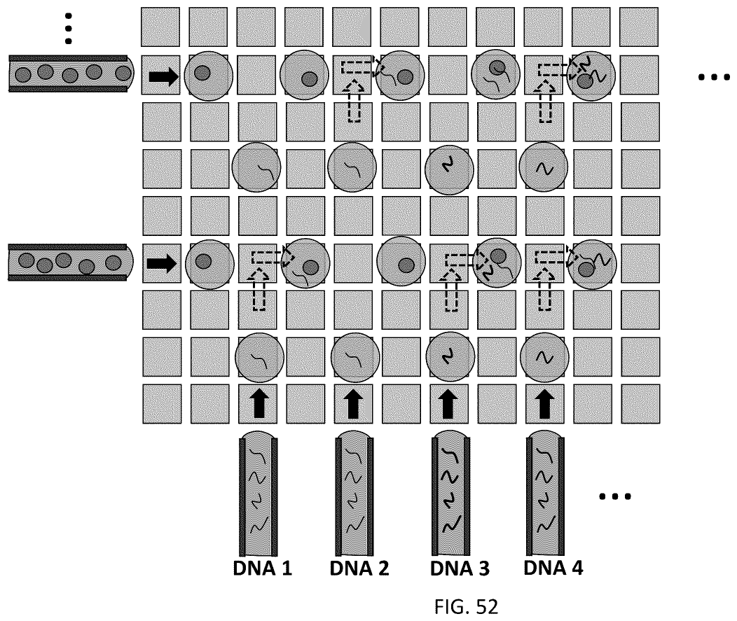

Another preferred embodiment of array-based parallel DNA payload deliver is indicated in FIGS. 51 and 52. There the purpose is to configure payloads and droplets for highly parallel and scalable delivery of multiple selected payloads into the cells, for combinatorial loading of payloads. In the indicated embodiment, cell droplet inlets are used to stage multiple cell packages in a distributed linear stream pattern, and multiple DNA payload inlets (bottom) are used to stage orthogonal payload droplets streams, and as shown in FIG. 52, as desired cell droplets can have payload droplets merged into them as they stream past the different payload options. In this way, as shown, as cell droplets move from left to right across the array, they can selectively be loaded with the optional payload DNAs, such that any combination of the optional payloads may be present in the cells.

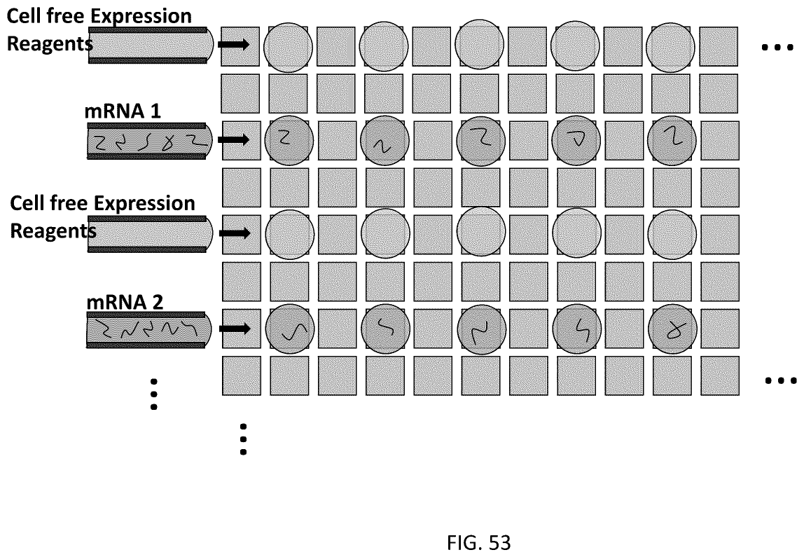

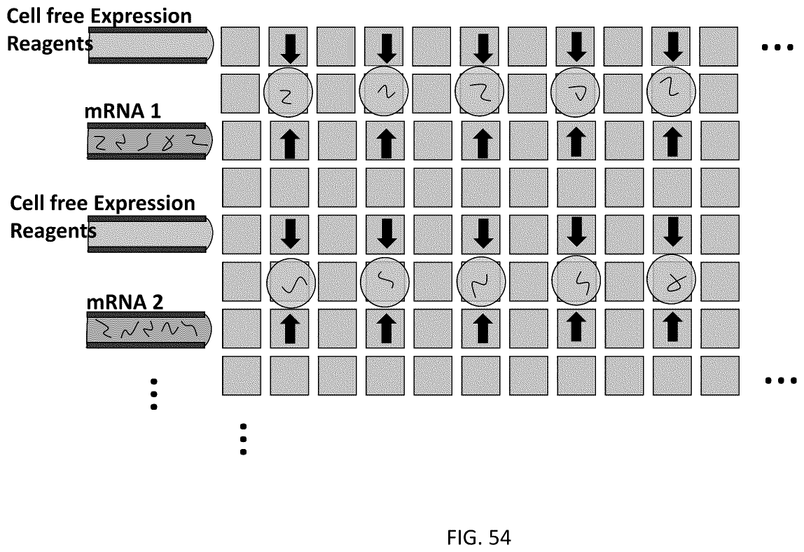

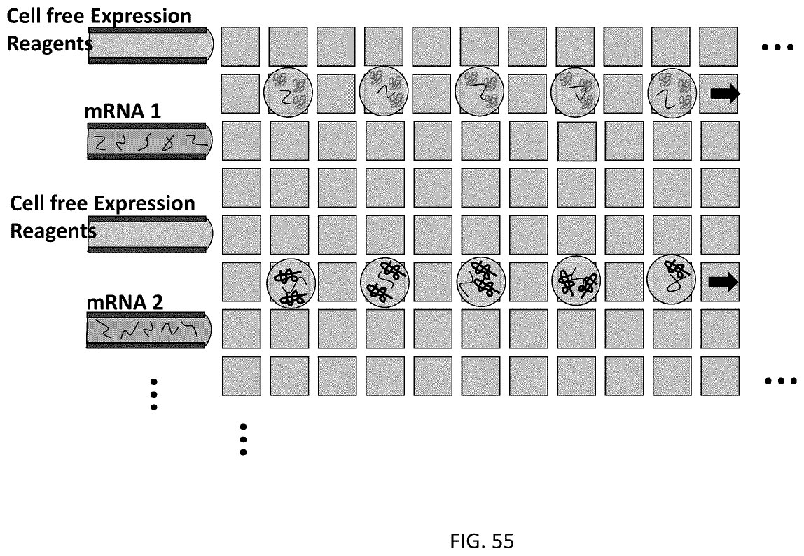

Another preferred embodiment is for the case of cell free expression, as shown in FIGS. 53, 54 and 55, in which the entire droplet dispensed from the indicated cell-free reagent droplet dispenser effectively acts as the cellular package as the target for the mRNA or DNA payloads. FIG. 53 shows staging of the reagent droplets and payloads, similar to FIG. 48. The next phase is shown in FIG. 54, where the payload droplets are merged into the reagent droplet “cells”, similar to FIG. 49. Finally, in FIG. 55 is shown the resulting protein translation. The net effect is to perform massively parallel and scalable cell free expression, driven by the mRNA or DNA targets of interest.

From these parallel payload packaging schemas disclosed here, and the prior disclosures of EWOD operations and cellular payload delivery methods, and it is obvious to those skilled in microfluidics and bioengineering that many obvious variations of such methods for moving and combining cells and payloads exist, along with many means of effecting the transfer of the payload into the cell, so as to achieve many diverse cell packaging workflows. All such obvious variations are intended to be covered by the exemplars disclosed here.

### EWOD Array Processor Architecture

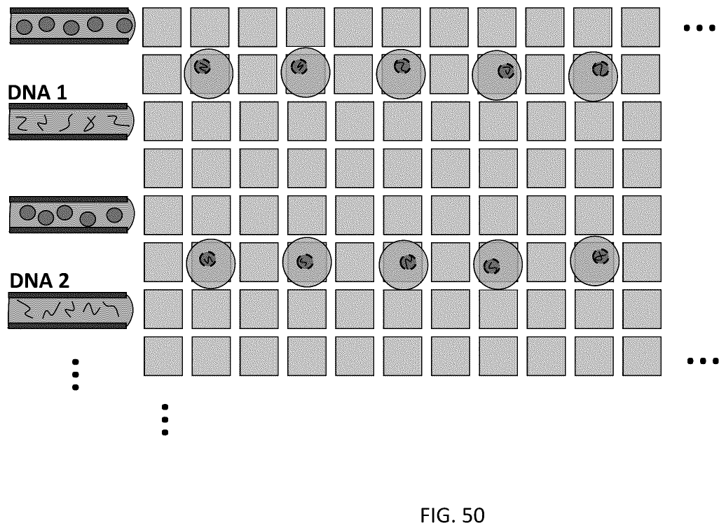

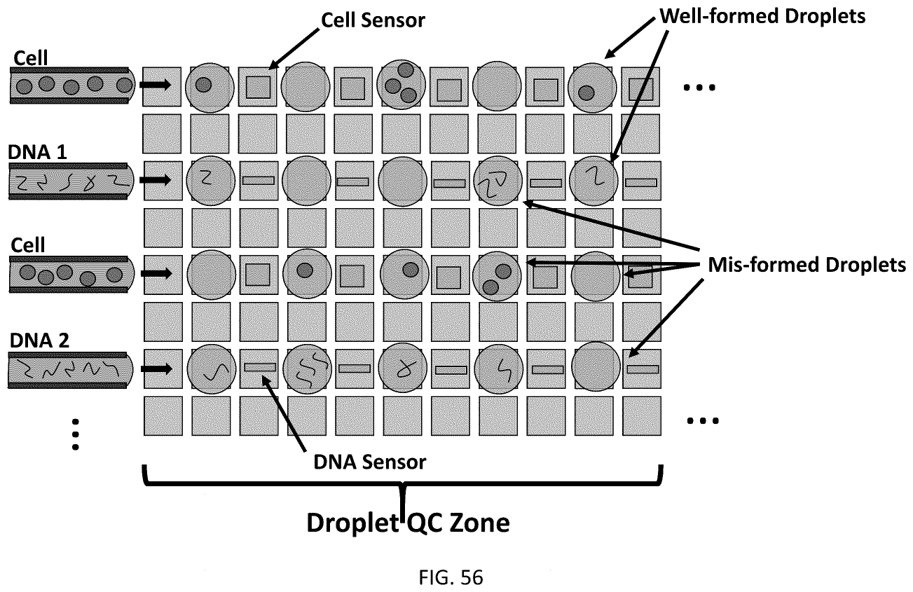

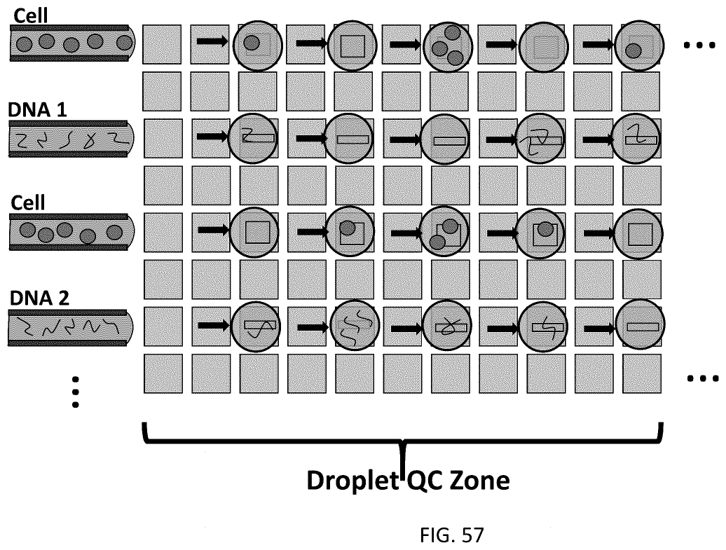

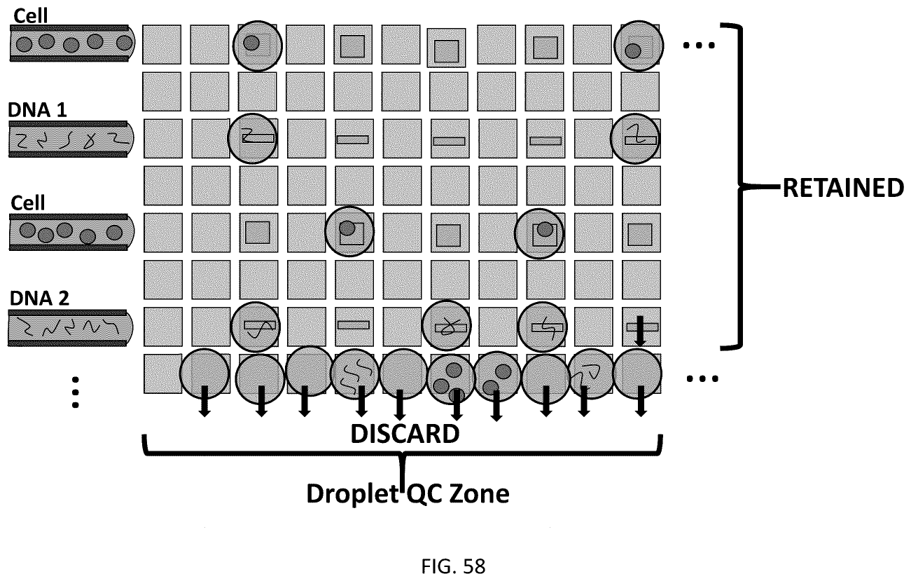

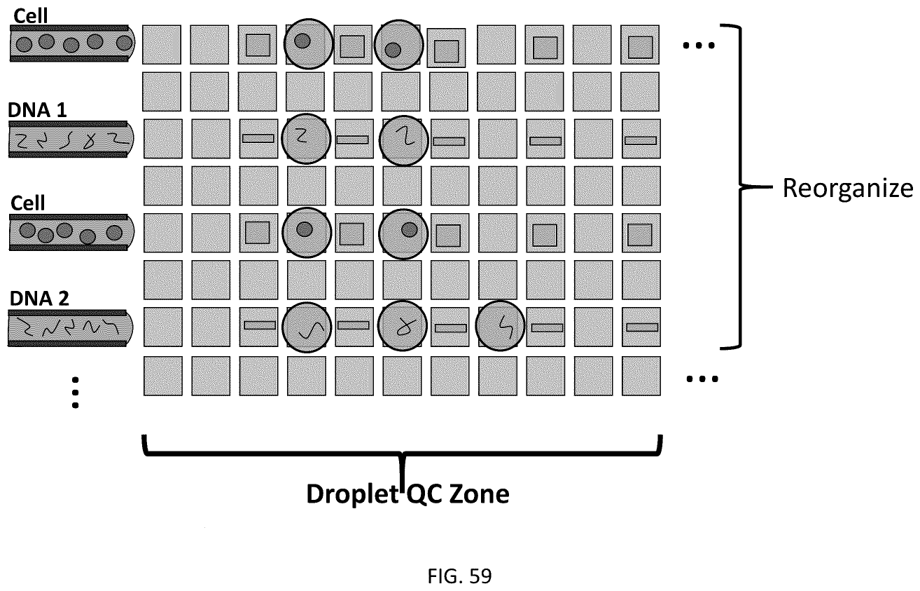

In is an object of this disclosure to provide high level architectures for the EWOD array that are capable of carrying out complex, parallel, scalable processes related to loading DNA payloads into cells and assaying the resulting cells for desired functionality, or measuring the cells for functional metrics. For the purpose of disclosing such architectures, there are major operational modules that can be organized into top-level complex array architectures. One such module is a Quality Control (QC) module, that is used to select well-formed droplets, and discard mis-formed droplets from the array. A preferred embodiment for such a QC module is illustrated in FIGS. 50 through 54. As indicated in FIG. 56, such a QC zone may be positioned at the site of droplet delivery to the array. As shown, there are inlets for cell droplets and DNA payload droplets. Well-formed droplets have content within desired tolerances. Indicated here is an example where well-formed cell droplets are specified to have one cell, and well-formed DNA payload droplets are specified to have a single DNA molecule. On the array as indicated are also sites that have sensors capable of detecting the cell count (indicated as square electrodes over wetting electrodes) and sites that have sensors capable of detecting the DNA payload count (indicated as narrow rectangular electrodes over wetting electrodes). As shown, droplets, as initially dispensed onto the array, may be well formed or mis-formed due to variability of the content of the inlet tubes. These candidate droplets are organized next to the appropriate sensor types. In the second phase, shown in FIG. 57, the dispensed droplets are moved over the respective sensor sites. At this point, the sensors are readout resulting in parallel detection of well-formed (green outline) or mis-formed (red outline) droplets. In the next phase, shown in FIG. 58, the mis-formed droplets are moved to a discard zone, and then discarded from the array. The well-formed droplets are retained on the array. Finally, as shown in FIG. 59, the well-formed droplets can be compactly reorganized for subsequent efficient and uniform motion processing on the EWOD array. Such a QC zone as indicated here may also, in preferred embodiments, by placed elsewhere on the array, to apply a QC filter to the droplets whenever such characterization and re-organization is required for a process of interest.

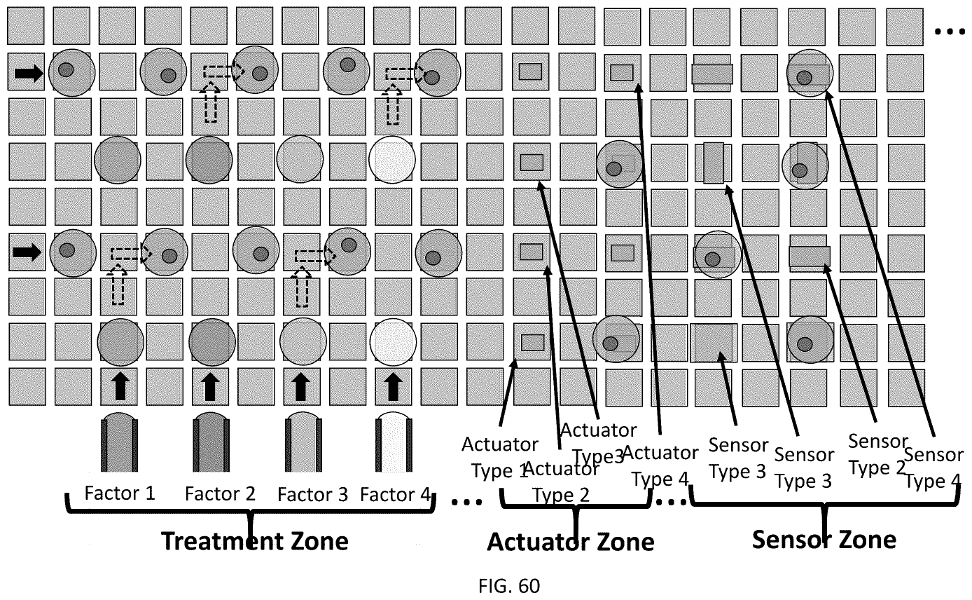

Additional array architecture modules are indicated FIG. 60, which has a Treatment Zone, where different chemical treatments and reactions are applied to cells, as well as an Actuator Zone, where droplets can be positioned over actuators to further effect the cell stimulation desired. Finally, there may be a Sensor zone, where droplets may be positioned over sensors in order to measure desired functional characterization metrics. Cells may pass through these zones in series, and may also cycle through such zones, as many times as desired. From this disclosure, it is obvious to those skilled in microfluidics and bioengineering that such zone can be configured into many possible high level EWOD device architectures, supporting many processes of droplet production, cell payload delivery, treatments, and functional assays and readouts. All such obvious variations and combinations are meant to be implied by the exemplars shown here.

Some embodiments may utilize portions of the chip as dedicated regions for cell incubation or maturation following steps of packing and/or actuation. These maturation zones may be comprised largely of actuators and sensors in close proximity to facilitate near continuous actuation where needed and monitoring of the packaged constructs in this state. In some embodiments, an ongoing QC process may be used during the maturation phase, for example to determine whether transcription, translation, expression, or cell growth is occurring as expected. In other embodiments, the maturation may occur on a separate chip or device mated to the packing EWOD chip, which may be configured similarly with actuators and sensors or merely act as a holding chamber while the desired screening processes occur.

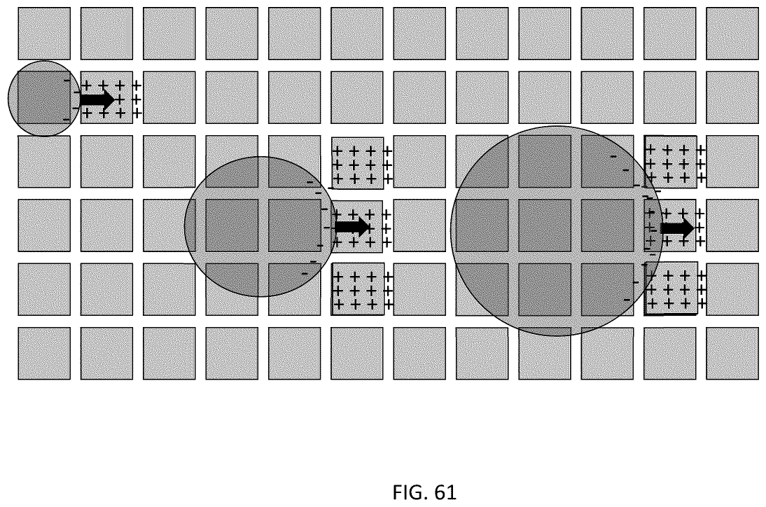

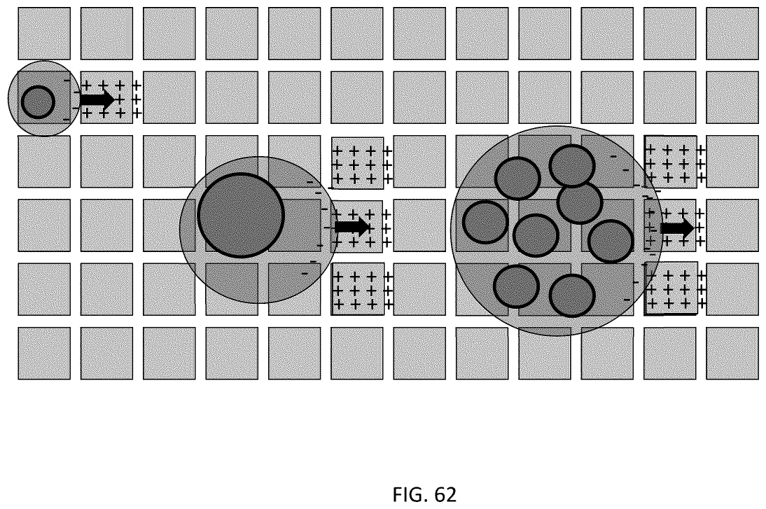

FIG. 61 illustrates that with a high-resolution grid of wetting electrodes, many sizes of droplets can be moved under voltage controlled wetting on the array, from the smallest such droplets whose size corresponds to the size of primary electrodes, to larger droplets which cover multiple electrodes and which can be moved through the coordinated activation of multiple electrodes as indicated. Such larger droplets may span 1, 1.5, 2, 2.5, 3, 4, 5, up to 10, or up to 100 or more electrodes, in droplet diameter. FIG. 62 illustrates that such larger droplets in preferred embodiments may be used to move larger cells that would not fit in the single-electrode droplets (middle), or multiple cells in a large droplet (right). This provides support for a great variety of droplet sizes on a fixed resolution electrode array, configured at the finest scale of interest for droplets. Such groups of small electrodes can also drive larger droplets to have shapes other than circular, such a rectangular or tear-shaped, by making use of a correspondingly shaped collection of small electrodes to transfer the shape to the droplet, which may be useful for various fluidic processes. Similarly, it is clear that a specially shaped electrode (rather than a composite shape formed as a set of smaller electrodes) could also be used to distort droplets into non-round shapes, such as might be used to pinch off droplets from a larger drop that has been distorted into a tear drop shape, for example.

### Integration of EWOD Arrays With DNA Delivery Devices

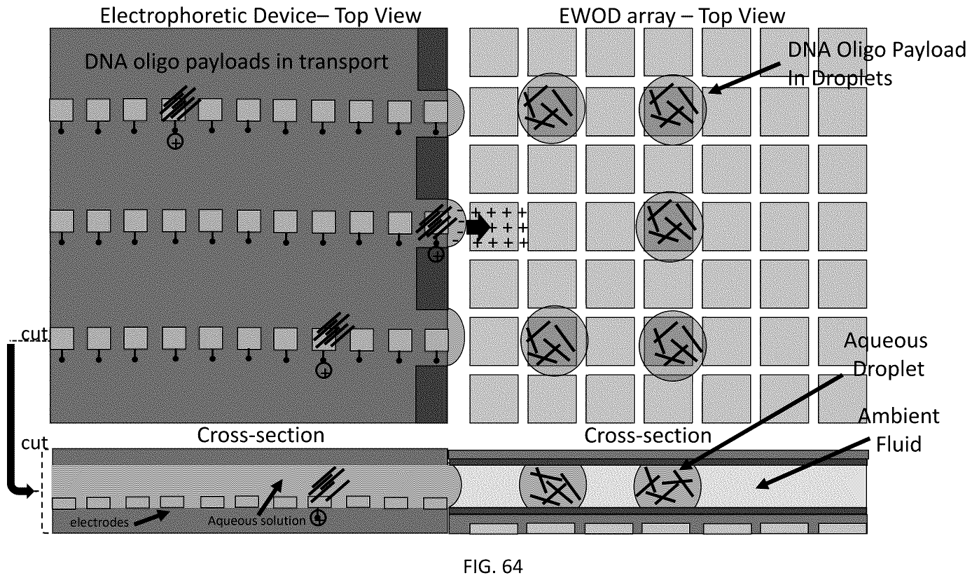

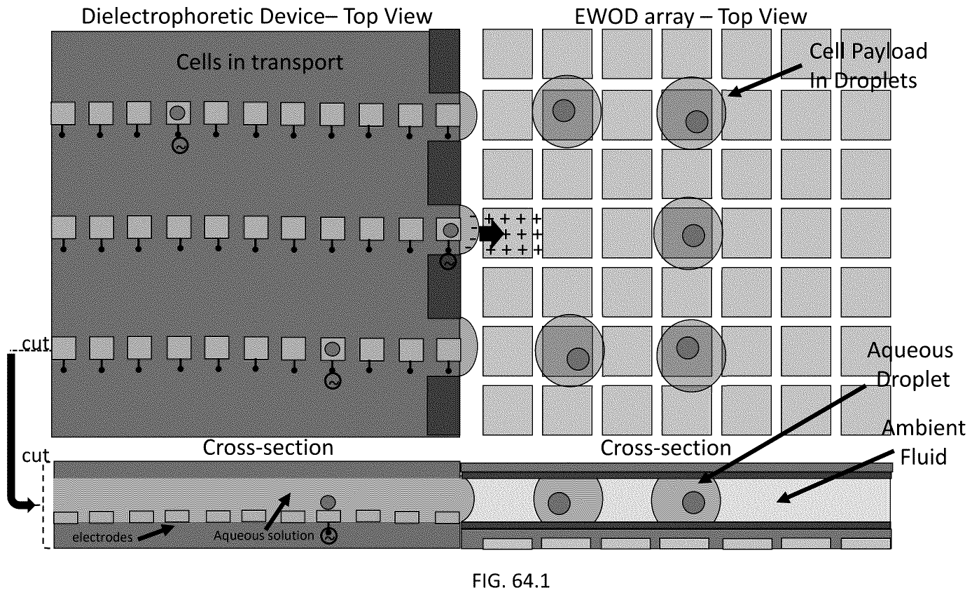

In preferred embodiments, The DNA payloads may be originating from sources in which DNA resides in nano-channel devices, or in devices which move DNA fragments by electrophoresis. Such devices may represent the devices on which the DNA payloads of interest are created through synthesis processes, for example, or may simply represent transfer processes for the DNA materials. FIG. 64 shows an integration of an EWOD array device to a DNA nanochannel device, in which long DNA molecules are being transported through nanochannels under voltage control. As shown in the bottom cross-sectional view, the nanochannel device can be physically mated to the EWOD device, such that the aqueous phase in the nanochannels can be pulled onto the EWOD array to form droplets (in the ambient fluid, such as an oil), with such droplets containing the long DNA payload molecules, which are contracted into the droplet staging area at the end of the channel, as a result of the voltage directed attraction of the payloads to the droplet staging sites (the middle channel is at this phase, and in the process of dispensing a droplet containing DNA). The result is that droplets are dispensed onto the array, containing the long DNA payloads.

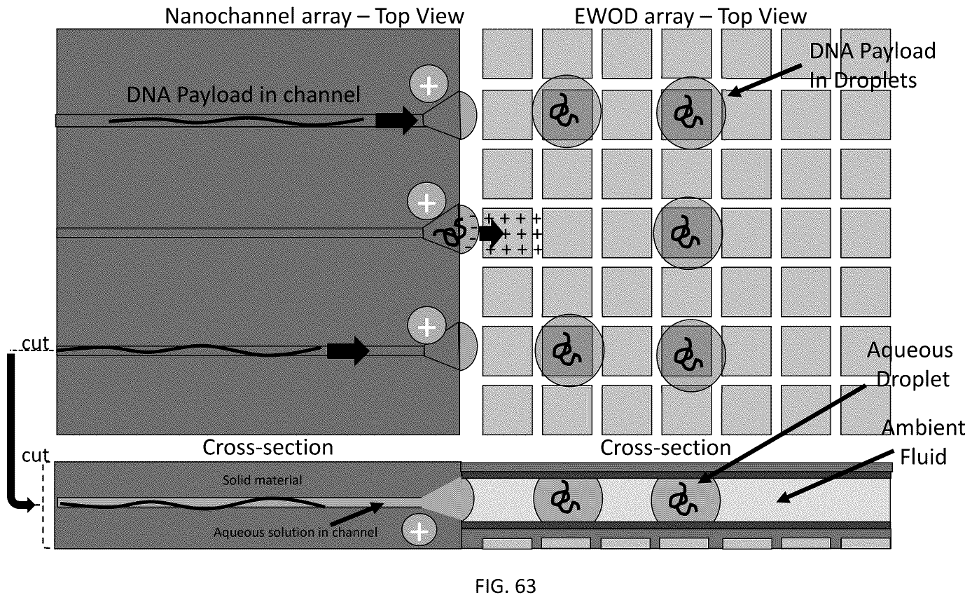

Note the time reversed process of that shown in FIG. 63 is also viable and illustrates a method of taking droplets with DNA payloads off the array, and loading the DNA payload into nanochannels, for robust transport to other sites for other processing.

FIG. 64 shows another preferred embodiment where the DNA motion device is using series of electrophoresis (or dielectrophoresis) electrodes to hand off DNA fragments from one electrode to the next, under the action of the electrode voltages attracting the DNA from the adjacent site. As shown in the lower cross-sectional image, this can be physically mated to the EWOD device by having the edge of Electrophoretic device sealed (dark bars on edge top view) except for open ports that define droplet exit ports. Physically mating this, with the ambient liquid phase of the EWOD device (such as oil) supplying the physical contact, enables the electrophoresis process to hand off the DNA to the final electrode at the droplet dispensing site (as shown in the middle lane), such that the DNA is dispensed into the droplet as it is extruded onto the array. The net result is that droplets are dispensed onto the array, containing the short DNA payloads.

FIG. 64.1 shows a preferred embodiment where a cell loading device uses a series of dielectrophoretic electrodes to hand off cells from one electrode to the next under the action of one or more AC signals applied in any variety and/or sequence of electrode configurations. Cells may be loaded onto the dielectrophoretic electrodes for organizational pre-and/or post-processing operations including, but not limited to, the phenotypic sorting of mixed cell populations, the removal of non-viable cells, and/or the isolation and transport of desired cell quantities.

Note the time reversed processes of those shown in FIG. 64 and FIG. 64.1 are also viable and illustrates a method of taking droplets with DNA or cell payloads off the array, and loading the DNA or cell payload onto the electrophoresis or dielectrophoresis electrodes, for robust transport to other sites for other processing.

It is a further object of disclosure, that as is made clear by the forward and reverse processes above, such EWOD arrays can be used as a transfer buffer between DNA nanochannel devices and DNA electrophoresis devices, so that a transfer of DNA material can be achieved from one such device to another, by passing through a phase of DNA payloads on droplets moving on an EWOD array device. This provides an efficient, scalable and massively parallel way to transfer material between such DNA motion devices.

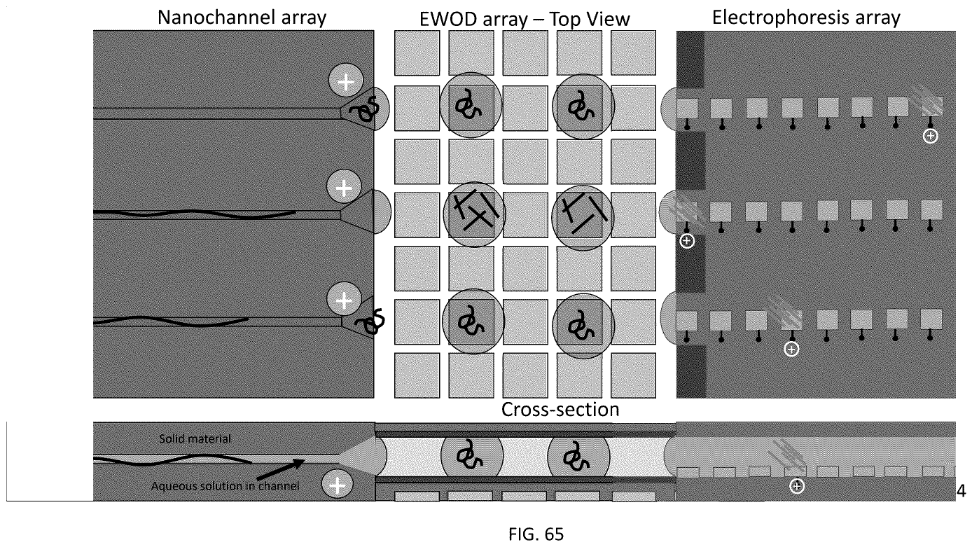

It is an object of this disclosure that EWOD array devices can be used to provide the interface between devices that internally transport DNA using electrical forces on free DNA in solution. One preferred embodiment is illustrated in FIG. 65, which shows an EWOD array as a bidirectional material transfer interface between a nanochannel DNA transport device, and an electrophoresis DNA transport device, where in either direction droplets carrying DNA payloads are extruded onto the EWOD array, transported across the array to the other device, and exported off the array into that device, transferring the DNA payload in the process. It is obvious from this disclosure to those skilled in microfluidic that many variations on such EWOD interface configurations are possible, and many variations are possible that incorporate the functionality of EWOD arrays as disclosed, such as the inclusion of QC modules, Actuator or Sensor modules. All such obvious variations and combinations are intended to be included in the present disclosures.

### CMOS Chip Devices

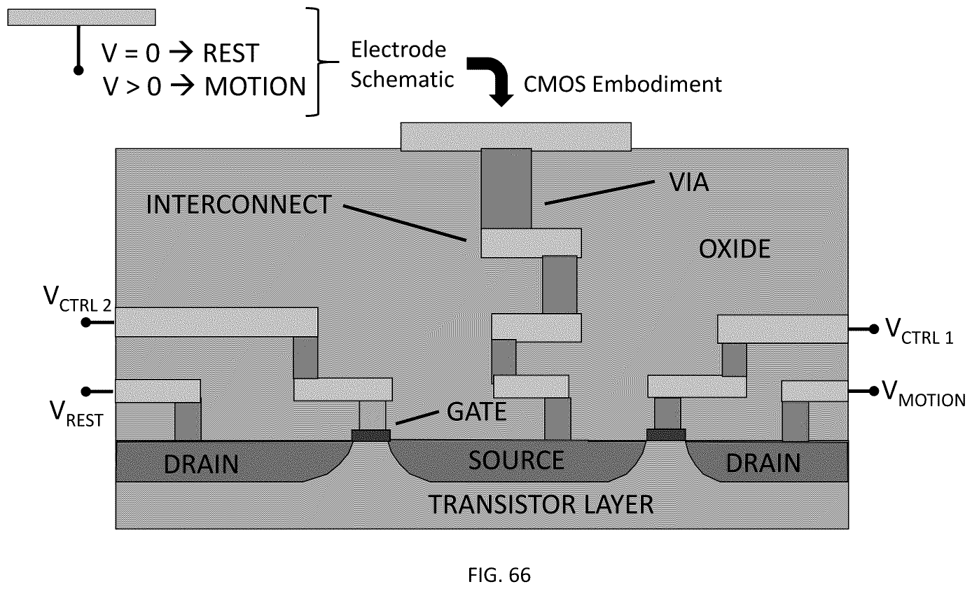

The EWOD electrode array devices disclosed are electronic devices. It is an object of this disclose that a preferred embodiment for these EWOD array devices is as a semiconductor Integrated Circuit (IC) chip device, and more preferably as a Complementary Metal Oxide Semiconductor (CMOS) chip device. Such CMOS chip embodiments have the benefit that they can leverage a substantial global manufacturing and supply chain infrastructure that has developed to produce CMOS chips and systems using these chips. FIG. 66 discloses one preferred and illustrative embodiment of the EWOD wetting electrode that has been disclosed herein, implemented as a CMOS device within a CMOS Integrated Circuit chip. Considering as show, a wetting electrode which is indicated schematically as being set at “V=0” (to bring a droplet to “REST”) or at “V>0” (to set a droplet in “MOTION”). This basic schematic of the electrode could be embodied in the CMOS material layer stack-up as shown: in this embodiment, the upper electrode would be a metal (either deposited within the CMOS foundry, or post-processed on top of a VIA layer produced at the CMOS foundry), which is connected down to the transistor layer of the CMOS by a series of Via's (vertical CMOS wires) and interconnects (horizontal CMOS wires), going down to make contact to a transistor lead, shown here as the source lead of the 3-terminal device (source/gate/drain), although it could as well be a source electrode in other embodiments. This connection to the MOTION or REST voltages is gated by the gate terminals shown at left and right, where the applied control voltages V_{CNTL 1} and V_{CNTL 2} can be used to open the connection to either desired external voltage. These controlled switches are implemented as transistors, at the transistor layer of the CMOS stack. From this disclosure, it would be obvious to those skilled in CMOS chip design and electrical engineering, that many variations on this embodiment are possible, that provide an implementation of the specified wetting electrode schematic functionality. All such variations are meant to be implied by the present disclosure. In a preferred embodiment, the top-level electrode—the wetting electrode—would be manufactured as part of the CMOS material stack and process at the CMOS foundry, to provide maximally integrated manufacturing. In other preferred embodiments, the top electrode may be added to the CMOS chip from the CMOS foundry, in a post-processing step carrier out separately from the primary CMOS chip fabrication. In such post-processing steps, the final electrode could be composed of a greater diversity of materials, such as diverse metals, or doped semiconductor material, or other materials that can function as a conductive electrode for supplying the required electrowetting voltages.

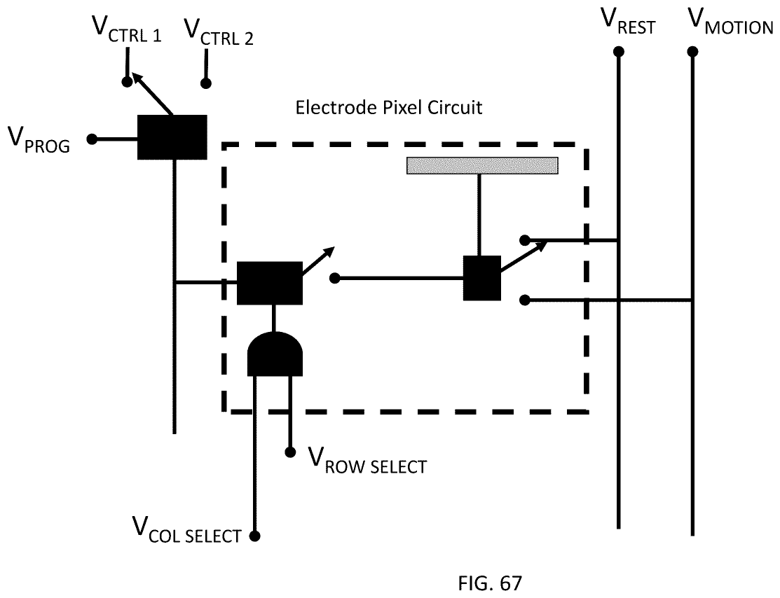

In the design of CMOS chips, it is common to provide the design via schematic circuits, which are then translated into CMOS fabrication design rules by automated or semi-automated design software. In the case that the chip has an array structure, with a rectangular array wherein the same circuit (“pixel circuit”) is repeated many times, there is provided a circuit schematic for one such instance of the circuit, also indicating how it connects to global control or supply lines. One such embodiment of a CMOS circuit schematic is shown in FIG. 67 which indicates a pixel circuit for a CMOS chip embodiment of an EWOD wetting electrode array devices such as has been disclosed herein. In this pixel circuit shown, the electrode can be connected via a switch to global supply lines providing the REST and MOTION voltages. The logical connectivity of this circuit is as follows: this switch is controlled in part by the ROW and COLUMN SELECT lines, which are used to address specific electrodes in the rectangular row x column array of electrodes. As indicated in the schematic, when both ROW and COLUMN SELECT are active, their AND is active, the control line of the electrode switch is connected to the V_{CNTL 1}/V_{CNTL 2} selection switch located off array, which is in turn controlled by the off-array control voltage V_{PROG}, that is used to program the pixel electrode to be in the REST or MOTION voltage state. In operation of this circuit, to put a particular electrode located at row R, column C in the array, in either the REST or MOTION state as desired, the programming voltage is applied, V_{PROG}, with the appropriate logical 0/1 value to select control voltage V_{CNTL 1} (for REST) or V_{CNTL 2} (for MOTION), and then the column select line for column C is activated, as is the row select line for row R, thus selecting the desired electrode pixel, and coupling its internal switch control input to the external line for the V_{CNTL 1}/V_{CNTL 2} selection. This in turn sets the internal pixel switch to the desired REST or MOTION voltage state. From this disclosure, it would be obvious to those skilled in CMOS chip design and electrical engineering, that many variations on this schematic embodiment are possible, to achieve the same function ends, that provide an implementation of the specified wetting electrode schematic functionality disclosed, or similar functions. All such variations are meant to be implied by the present disclosure.

### EWOD Chip Systems

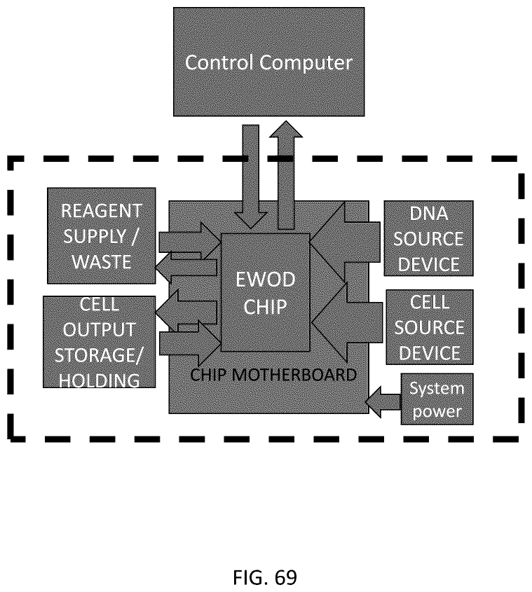

It is an object of this disclosure to describe present embodiments for the complete system required to perform applications disclosed herein, on EWOD array chip device as disclosed herein. For applications comprising the packaging of DNA payloads into cells, and subsequent functional assessments on the resulting cells, a system level architecture of a complete system is shown in FIG. 69. As indicated there, a master control computer provides the program that controls the EWOD array chip, in terms of the timing of activating all electrodes, sensors and actuators on the EWOD chip at the appropriate times, and employing the appropriate logic. This computer also receives the information off the EWOD chip, such as the outcome status indicators for all operations performed, including any execution exceptions, or error conditions resulting, as well as the outputs of all sensors on the chip that acquire sensor data, such as pertaining to drop motion, and the biochemical state of the droplets, including the results of the functional measurements on the cells and droplets on the array chip. The EWOD chip itself is mounted on a motherboard, which supplies standard electrical functions such as power supply, supply voltages and currents, supplying system clocks, and providing the data bus connections and control bus connections to the chip. This chip is physically mounted on the motherboard, typically relying on an interposer Printed Circuit Board (PCB) as the primary physical mounting point for the chip, such as by wirebonding and gluing to the interposer. The motherboard also connects to a system power supply to power all on-board operations. The reagent supply to the EWOD chip, supplies all the diverse biochemical reagents required, including the ambient fluid phase used to load the chip with fluid initially, such as an oil, as well as the aqueous phase solution, as well as other reagents required for the functional assays, such as the reactants for various reactions, or stimulating factors for the cells, or washing solutions to clean out the EWOD chip for further re-use or storage. There is also a waste stream from the chip to the reagent supply, to contain waste materials coming off the EWOD chip.

For the primary application of delivering payloads into cells, there is one source for the DNA payloads, and another source for the cell materials. These physically transfer their contents to the EWOD chip through the indicated physical interfaces, which embody the other disclosures herein relevant to this subsystem.

Finally, the result of the EWOD chip cell packaging and functional assessment may include cells with desirable properties, that are to be saved for subsequent processing or characterization. Therefore, there is a cell output storage unit, where such cells can be collected and stored in a convenient format, such as in well plates, under appropriate storage conditions. In some preferred application embodiments, such cells may be held off-EWOD chip temporarily, and then transferred back on chip for subsequent processing. In preferred embodiment, this could for example be for the purpose of growing up the cells, and then returning all or a portion of the grown cells back to the array chip for additional processing and assessments. Or this could be for the purpose of performing off-line assays, such as genome sequencing, on such cells, prior to returning them to the array chip for further processing.

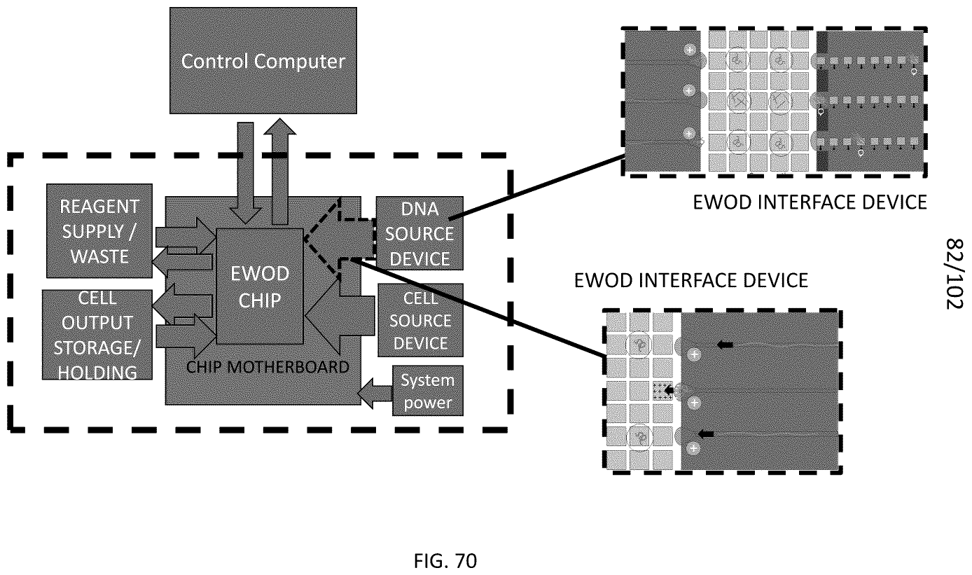

As shown in FIG. 70, in some preferred embodiments, the indicated subsystems may themselves contain EWOD chip devices to perform dedicated fluidic functions within those modules, such as converting between DNA handling devices (e.g., nanochannel and electrophoresis) within such modules, or converting format from a DNA handing device to an EWOD chip array format, which may then mate to the main EWOD chip array to fulfill the delivery function indicated. Similarly, the cell output/holding subsystem may itself utilize EWOD chip devices. Such EWOD devices may in preferred embodiments be used to hold cells for the purpose of incubation at a fixed temperature, in conjunction with temperature actuators, so as to incubate cells for cell growth, for extended periods of time. Subsequently such grown out cells may be returned to the main EWOD chip for processing. For example, embodiments of the MAGE process may require multiple cycles of such cell growth incubation phases. For example, in other preferred embodiments, the cells may be stimulated to grow into small organoid cultures, either within droplets, or at electrodes or zones where such organoids my adhere or remain non-adherent. In preferred embodiments, organoids may be seeded by bringing together different cells variants in different droplets to seed complex cell populations. Such organoids would preferably be less than 1 mm in diameter, and preferably 100 microns in diameter or less, and this has the benefit of enabling organoid engineering on the platform.

### EWOD Extended System Architectures

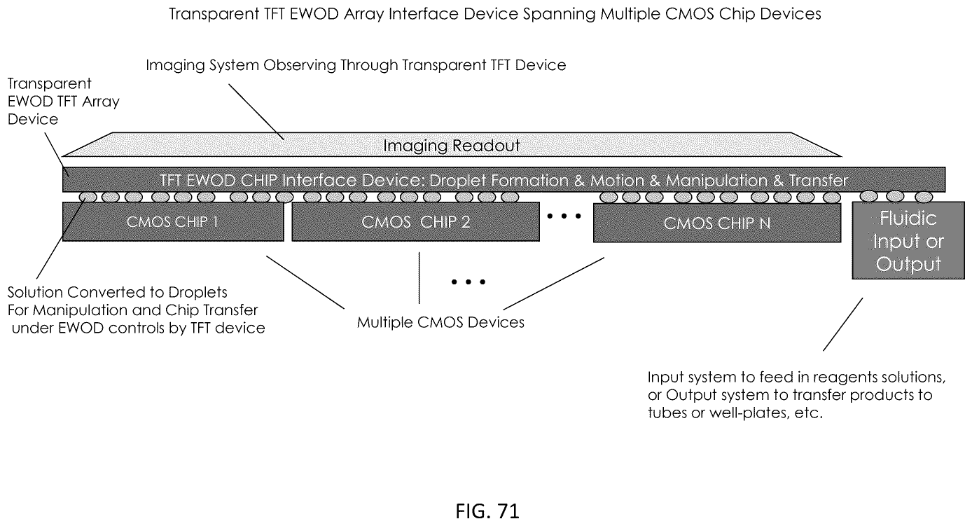

The following embodiments illustrate various obvious extended system architectures that result from obvious combinations and compositions of the devices and systems disclosed above, and in particular, from those disclosures as in FIG. 70, at the system level there are there are many obvious variations and combinations that may be used to create extended systems comprising which multiple chip devices, including many obvious ways of fluidically connecting these chips, as disclosed in FIG. 70. In particular, note that in these extended systems, it is readily possible to mate two different EWOD chip devices so that they can pass droplets between them, through direct physical contact or abutting of, or overlapping of, such devices, as disclosed in FIG. 70. More generally, preferred system embodiments may comprise EWOD devices and other fluidic chip devices which must be integrated fluidically, and which also must further be integrated to other subsystems allowing input and output (I/O) of fluids, such as for reagent input, waste output, and recovery of cellular or other products produced by the system. In addition, preferred system embodiments must be integrated with electrical I/O for purposes of power, control, and sensor readout, and potentially with optical I/O, such as for an imaging system that reads fluorescent outputs or optically monitors the positions and movement of droplets, cells, or DNA. Various preferred embodiments for these extended system architectures are shown in FIGS. 71 through 78. Based on these disclosures illustrated there, those skilled in the art of system engineering or instrument engineering would understand that many additional variations and combinations of the features shown here are possible, and these and all such obvious extensions are meant to be encompassed by the examples disclosed here. In particular, there may be many suitable configurations where the distance between the faces of two opposing surfaces varies according the needs of a given application, and the disclosures here are intended to encompass those versions, as well as instances where a mechanical system can vary the distance between the surfaces as needed.

FIG. 71 shows a preferred embodiment of an extended system, in which a TFT EWOD interface device of the type disclosed in FIG. 70 is used to spanning multiple functional chips, and output devises, providing a means to transfer fluids from on chip device to another, or to the output devise (such as transfer to standard or custom well-plates, where each well receives on or multiple droplets). The advantages of a TFT array device for this EWOD interface device are foremost that it can be of a large area (—while still providing high pixel density, such as pixel pitches in the range of 10 microns to 200 microns, much higher than PCB devices, although not as high as the limits of CMOS chip devices), since TFT array devices are readily fabricated at much larger areas than the die size of CMOS chips (which are limited to less than 10 square centimeters in area by the optical reticle size on photolithography systems). TFT array devices can have areas from tens to hundreds to thousands to even tens of thousands of square centimeters (as in large panel TV displays)—and thus TFT devices could readily be manufactured to a size that, as such a covering EWOD interface device, could span or cover multiple CMOS chips, even those that are made at a typical maximal die size of ˜10 square centimeters. In preferred embodiments such as indicated in FIG. 71, such a device could span 1, 2, 3, or more, or up to 10, or up to 100, or more CMOS chip devices, which might be laid out in a row, or in a two dimensional array. A second major advantage is that TFT array EWOD devices can be made transparent by using the methods commonly used in TFT display technology—such as fabricating the TFT on a transparent glass or plastic array and using Transparent Conducting Electrode (TCE) materials that can render electrodes and electrical connections transparent. By making such a transparent EWOD interface device, an imaging system can be provided that can send light into, and read light out of, the solutions and materials between the interface device and the substrate. In particular, such an imaging system can be used to excite fluorescence, and read out fluorescent reports of biological processes occurring on the device, such as for the readout of fluorescent reporter assays used to assess function, and can also be used to visually track droplet movements, or movements of labeled DNA or proteins or cells, to monitor the system performance. In general, in various embodiments such an interface device could also be constructed all or in part or comprising PCB or CMOS EWOD devices as well, but such PCB and CMOS devices do not as readily support the desirable combination of (i) large area, (ii) high density pixels (iii) optical transparency, and (iv) standard design and mass-manufacturing processes and capacity as do TFT devices. Further, while FIG. 71 shows the interface device as an upper surface, there is no implied direction of gravity there, and that could be an arbitrary direction relative to the illustration, so in general the EWOD interface device could, in particular, be a lower or sideways facing surface (relative to the direction of gravity).

As indicated in FIG. 71, the TFT EWOD interface device can serve as the means to transfer fluid between different CMOS chip devices. For example, such CMOS chips could include (one or multiple) chips that perform DNA synthesis, DNA assembly (such as joining oligos to form genes), DNA long assembly (such as joining multiple genes together, to form chromosome-scale segments), cell packaging of DNA payloads, and cell (or cell free) functional testing. As indicated, such a TFT interface device can also provide an interface to fluidic inputs, such as the input of reagents or cell-carrying solutions, and fluidic outputs, such as the output of waste solutions, or of products of the bioengineering, such as proteins or cells of interest. Such products may, in preferred embodiments, be transferred to tubes, well plates, or droplet arrays on preferred analytical substrates, such as glass microscope slides, mass spectrometry matrix substrates, or electron microscope grids or substrates. In addition to providing liquid transfer or I/O, in preferred embodiments the TFT interface device can also be used to perform other desirable fluidic operations in the context of such a system, such as droplet formation or generation, droplet manipulations (such as splitting or merging or mixing), and useful droplet-based processing, such as processes for performing DNA synthesis, performing DNA oligomer ligation/joining operations (of any length of oligonucleotides from 2-mer oligonucleotide segments on up through 300-mers), assembly of longer DNA constructs (to reach lengths of up to 1 kb, up to 10 kb, up to 100 kb, or up to 1 Mb of longer), methods of error correction or error elimination to remove errors from target DNA constructs, performing PCR or other biochemical reactions or processes, and delivery of DNA payloads into cells using chemo-, electro-, or mechanical poration to bypass cell membranes, high-throughput screening of drug or chemical libraries against cellular or other targets, or the culturing of larger cell populations or organoids within droplets.

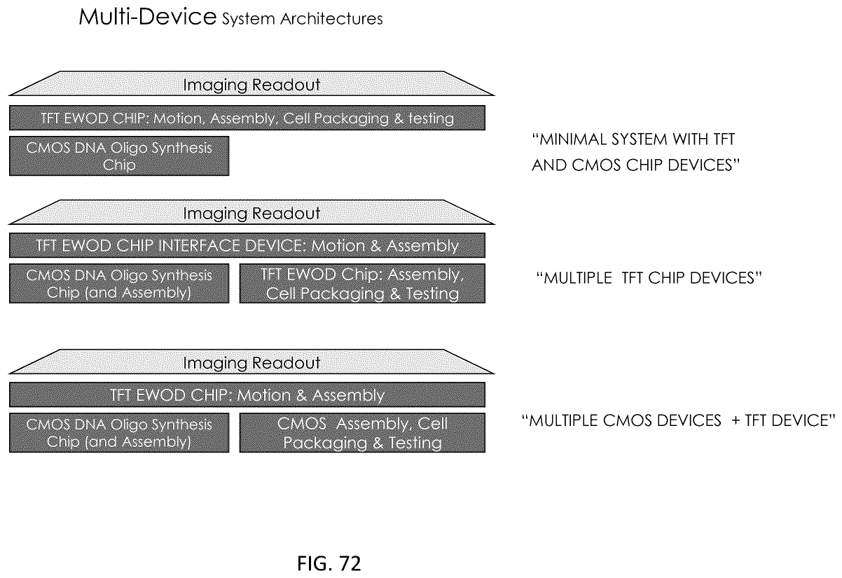

FIG. 72 shows various preferred embodiments for a TFT EWOD interface device system. In the upper embodiment, a TFT EWOD interface device covers a single CMOS chip device that performs DNA oligo synthesis, and the TFT EWOD interface device supports movement off the CMOS chip, and performing the other operations of oligo assembly, long DNA assembly, DNA error correction or elimination, packaging of payloads into cells or cell-free expression droplets, and cell functional screening. The transparent TFT EWOD interface device also supports interfacing to an imaging system. This is a minimal embodiment, which carries out the entire cell engineering workflow with just a synthesis CMOS chip and a TFT interface device. The middle embodiment shows a TFT EWOD interface device covering a CMOS chip for oligonucleotide synthesis and possible assembly, and a TFT EWOD device as a second lower chip, further providing any of the diverse functions it may perform, such as DNA assembly, cell packaging and testing. The lower embodiment shows a TFT EWOD interface device covering a pair of CMOS chips, one for oligo synthesis and possible assembly, and another that is a CMOS EWOD device, further providing any of the diverse functions it may perform, such as DNA assembly, cell packaging and testing.

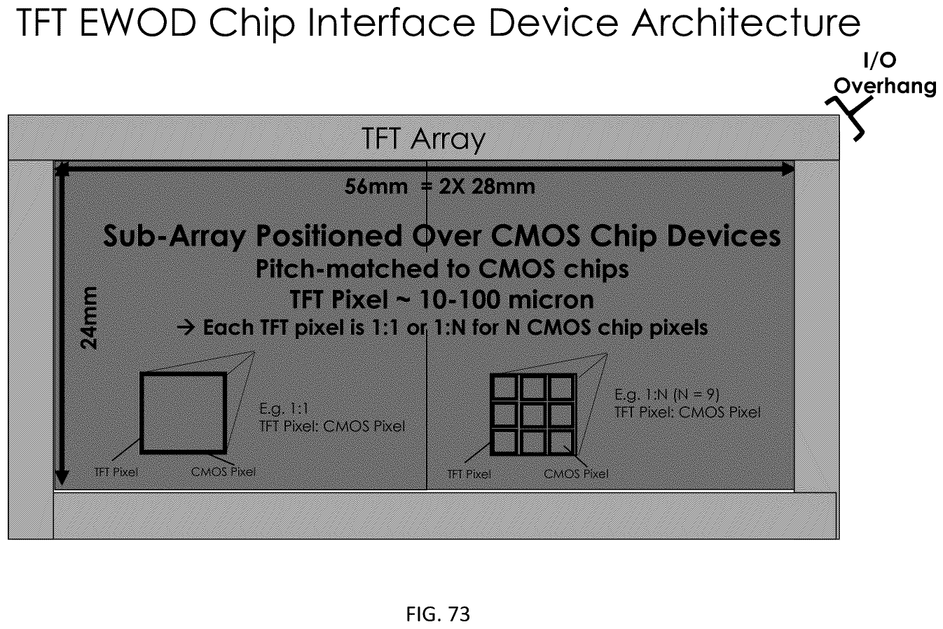

FIG. 73 shows one preferred embodiment of the TFT EWOD interface device, in which it has an area that extends beyond the area of the chips it spans, such that the overhang can be used for droplet manipulations related to fluidic input and fluidic output, by control movement of incoming and outgoing droplets from the marginal portions of the TFT pixel array. Example dimensions shown there (24 mm×56 mm) are not meant to be limiting, and just illustrate one example which is sufficient to cover two full reticle-sized CMOS die, placed abutting each other, each of which could be 24 mm×28 mm. In addition, in preferred embodiments as indicated in FIG. 73, the size of pixels on the TFT EWOD interface device array would be pitch matched to pixels on the CMOS chip devices, possibly 1-to-1 (shown at left) or possibly 1-to-N, for example the TFT pixel covering a N=3×3=9 pixel sub-array of the CMOS chip. In various embodiments, the number of CMOS pixels covered by 1 TFT pixel could be square sub-arrays of N=1, 4, 9, 16, 25, 36, 49, 64, 81, or 100, or more, or rectangular sub-arrays of any size, up to 10, up 100, up to 1000, or more.

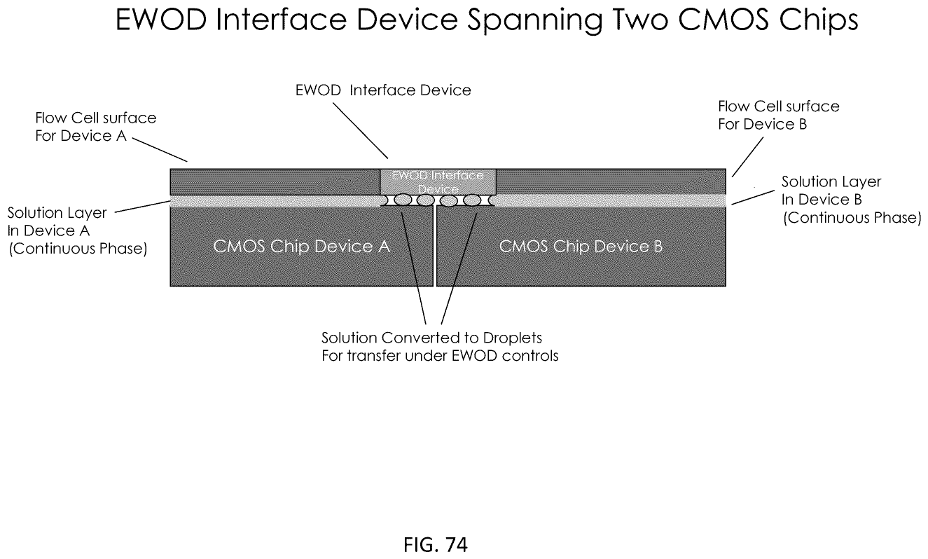

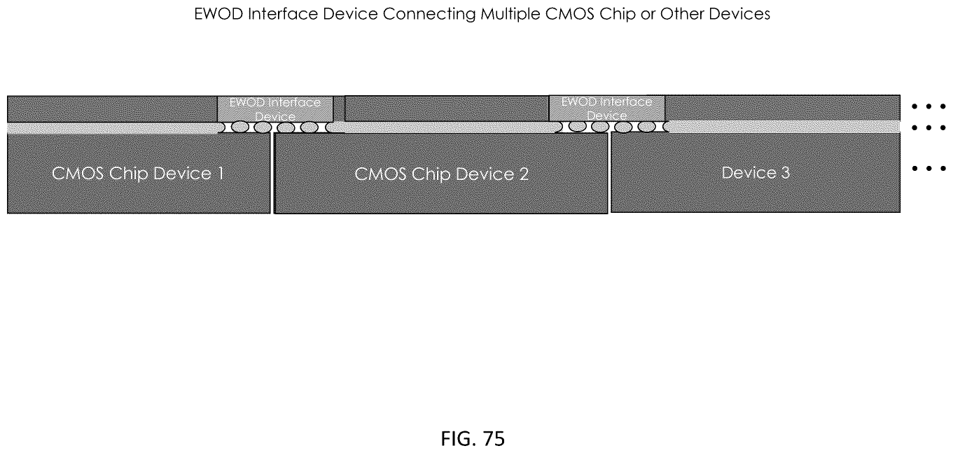

FIG. 74 shows another preferred embodiment for transferring solution between chip devices (or transfer between such devices and fluidic Input/Output subsystems). In this embodiment, as disclosed in FIG. 70, the EWOD interface device sits over the boundary between chips (or the boundary between a chip and a fluidic I/O devise), and acts to transfer fluid as droplets across device boundaries. The fluid may exist in continuous or droplet phases in the devices being interfaced, indicated in FIG. 74 as a left CMOS chip device A, and a right CMOS chip device B. In various preferred embodiments, the EWOD interface device may be a CMOS chip EWOD array device, a TFT EWOD array device, or a PCB EWOD array device. FIG. 75 illustrates the use of such EWOD interface devices to connect multiple CMOS chips or other devices. This EWOD bridge mechanism can be used to connect together any number of substrate devices that abut each other or are placed adjacent to each other, as indicated. Such devices being connected may be laid out end to end as indicated or may by laid out in a two-dimensional layout, with EWOD interface devices connecting across edges of adjacent devices.

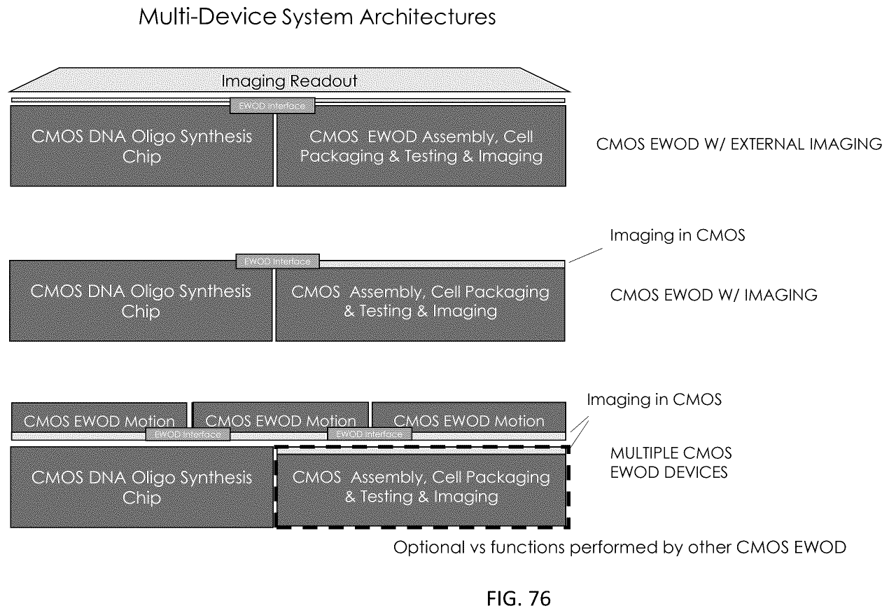

FIG. 76 shows preferred embodiments in which EWOD interface devices are used to connect together various chip devices. In the upper embodiment, an EWOD interface device connects CMOS chip devices performing various functions, and the remainder of the (passive) interface device is a transparent material (e.g. glass or plastic) such that an imaging system can engage the chip devices through this transparent interface device. The middle embodiment shows a configuration in which the EWOD interface device connects two CMOS chip devices, and where the imaging system is integrated into the second CMOS chip device (in the form of on chip optical or image sensor pixels) to provide the functions of readout of fluorescent assay reporter signals, optical observation of droplet motions, or other optical signal detection. The lower embodiment shown in FIG. 76 shows an EWOD interface device used to join together CMOS EWOD chip devices to span the other chip devices, overcoming the area limitations of individual CMOS chip devices. In the configuration shown, optical or image sensors are built into the CMOS EWOD devices, so that there is in effect an equivalent imaging system capability, as in FIG. 71, but here it is integrated into the CMOS chips. The various functionalities indicated in the bottom CMOS chip EWOD device can also in various embodiments be shared with or offloaded to the other CMOS chip EWOD devices, so that those do more than just motion and imaging. From these disclosures, it would be obvious to one skilled in the art of system design or instrument design that many variations and combinations of features shown in these EWOD interface device systems are possible, and the examples shown are intended to be in no way limiting, and all such obvious variations are intended to be encompassed by these examples.

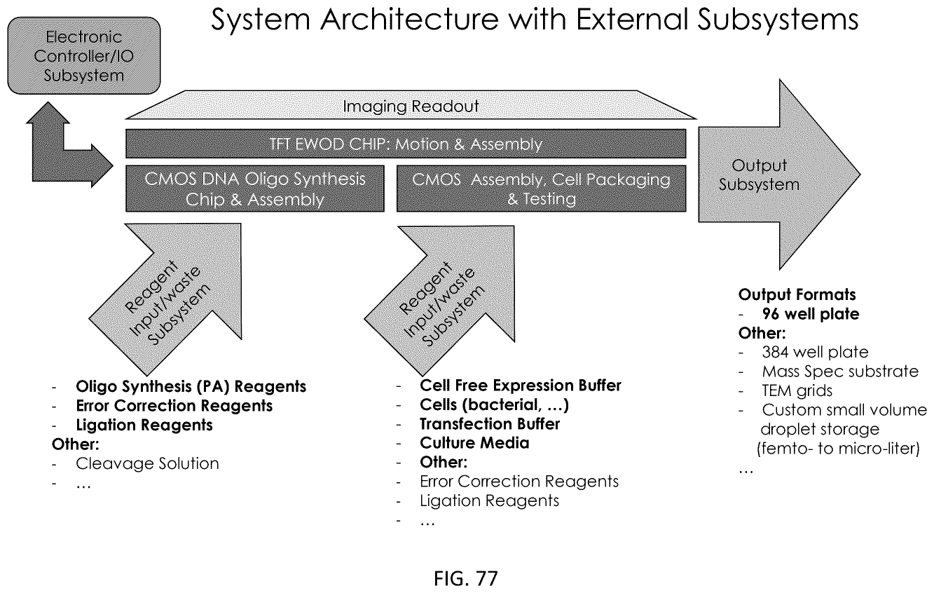

FIG. 77 shows preferred embodiments of the auxiliary subsystems that engage with a fully integrated EWOD system for cellular (or cell free) expression and functional screening. Shown is one such representative device architecture, and indicated are that it must integrate to an electronic I/O subsystem to that supplies control signals, power and voltage lines, and takes off sensor signals system evaluation measures and device status indicators. In addition, the various chip devices have reagent supplies and waste removal streams that must be integrated, typically different ones for each chip device. Representative such reagents are indicated in the figure. Finally, an output subsystem must be integrated to allow the products, such as proteins or cells, to be collected for storage or subsequent off-device processing. Such output may comprise placing fluids into standard tubes or well plates, or custom small volume storage arrays (for droplet volumes from microliter down nano-, pico- or femto-liter scale), or various analytical formats, such as spotting droplets or cells onto a glass microscope slide, a mass spectrometry matrix substrate, or electron microscope grid or substrate. From this disclosure, many detailed forms and variations for these auxiliary subsystems and their constituents and the primary device configuration would be obvious to those skilled in engineering system and instrument design, and this example is not meant to be limiting, and rather is intended to encompass all such obvious variations.

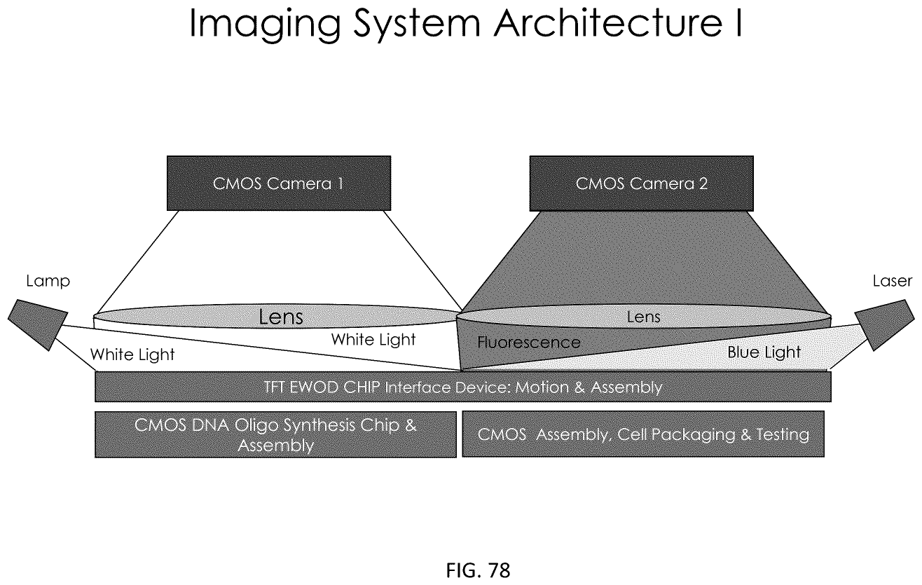

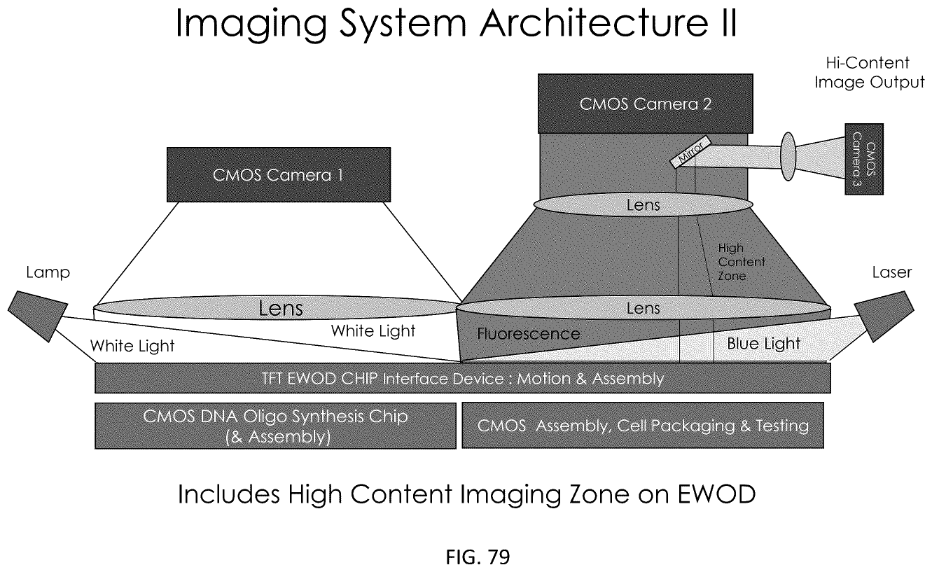

FIG. 78 shows a preferred embodiment for the imaging system that engages to system through a transparent TFT EWOD interface device. As indicated, one high-resolution CMOS video camera may monitor the first portion of the TFT array in white light, to track droplet motions, while a second high-resolution CMOS video camera may monitor the cell packaging and testing portion of the TFT array, using fluorescent imaging to read out the functional assay reporter signals. In such an optical system, the cameras preferably have the number of pixels needed to resolve individual droplets on the TFT array or CMOS EWOD array in each frame, and so such cameras preferably have at least 1 million, and preferably 10 million to 100 million or more pixels. Such images may be monochrome or full color in various embodiments. In other embodiments, the well-known imaging technique of Time Delay Imaging (TDI) may be used to reduce the number of image sensor pixels required to properly observe the observation area. FIG. 79 shows a further preferred embodiment in which there is a high-content imaging zone, which would enable high resolution imaging of cells in such a zone, to quantify report signals from individual cells, or possibly resolve features within cells, such as where a fluorescent reporter is localized at a subcellular level, or changes in cell morphology due to the cellular engineering, or to resolve features of groups of cells, such as counting living cells in the droplet, or observing the structure of a cell colony or organoid tissue that has grown at a location on the device. From these disclosed imaging system details, it would be obvious to one skilled in optics that many variations on such imaging systems are possible, and it is not the intent of this present example to be limiting, rather it is the intent that these exemplar imaging system architectures encompass all other such obvious variations and extensions.

### Experimental Demonstrations of Electrokinetic Methods for DNA Manipulation in Fluidic Devices

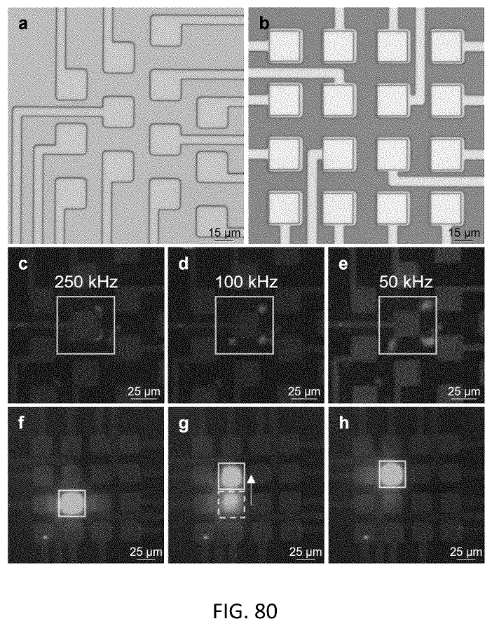

In the context of manipulating the DNA payloads to load droplets with payloads, or to form droplets with payloads, or to move payloads within droplets, such as bringing the payload to a definite location within a droplet or within a droplet generator, it is advantageous to be able to use electrical forces to transfer and localize such DNA payloads. For example, DNA electrokinetics could be used to increase the efficiency of cell packaging, by precisely positioning the DNA payload in close proximity to the cell, within the droplet. The demonstrations shown in FIG. 80 show the basic ability of various such electrical forces to transfer and localize DNA on a model electrode array. In this demonstration, dielectrophoresis and electrophoresis electrical forces are used to manipulate fluorescently labeled DNA molecules, using micro-electrodes fabricated on a silicon wafer substrate, with an external electrical controller driving the voltages applied to the various electrodes.

Two silicon-based devices shown in FIG. 80 were fabricated for these electrokinetic experiments. Device 1 was designed for alternating current (AC) electrokinetic manipulation of a longer fragment, 1 kilobase fragment with a sequence taken from the PhiX genome-derived DNA sequence. Device 2 was designed for direct current (DC) electrokinetic manipulation of DNA oligonucleotides (15 bases in length). Passivation layers were deposited onto a silicon wafer using plasma-enhanced chemical vapor deposition (PECVD). Electrodes and wires were formed using photolithography techniques. Briefly, a photoresist layer was subsequently deposited and patterned via photolithography. A platinum on chromium layer was deposited, and the photoresist removed via etching. A second passivation layer was then deposited by PECVD (Device 1) or atomic layer deposition (Device 2). Additional processing steps were necessary to fabricate Device 2. A second photoresist layer was deposited and patterned via photolithography. A titanium layer was deposited, and photoresist was removed in a subsequent metal liftoff. A third passivation layer was deposited via PECVD. A third photoresist layer was deposited and patterned via photolithography. A subsequent dry etch exposed platinum metal at the terminal points of each lead. The remainder of the photoresist layer was removed, completing fabrication of Device 2.

AC electrokinetic experiments were performed using Device 1, which presents planar silicon dioxide-passivated platinum electrodes (25 μm by 25 μm) spaced approximately 18 μm apart in a hexagonal array. Samples consisted of aqueous suspensions of PhiX DNA (1 kb) labeled with SYBR Gold and suspended in deionized water at a concentration of 1.5 ng/μL. Sample droplets were deposited onto the chip surface via pipetting. Electrical signals consisted of sine waves (Vpp=10 V; VDC=0; high impedance) of varying frequency (f=50-250 kHz) sourced by a Keysight 33612A waveform generator.

DC electrokinetic experiments were performed using Device 2, which presents planar platinum electrodes (25 μm by 25 μm) spaced approximately 18 μm apart in a square array. A porous agarose layer was deposited onto the chip surface to protect the integrity of the chip and sample during experimentation. An adhesive-based flow cell was assembled onto the chip. Samples were loaded into the fluidic chamber of the flow cell using a KDS Legato 110 syringe pump. Samples consisted of 10 nM aqueous suspensions of Cy5-labeled oligonucleotides (15 bp) with 50 mM L-histidine additive. Applied electrical signals consisted of DC voltages (VDC=2.5 V) sourced by a PalmSens4 source-meter unit in the chronoamperometry setting.

Silicon-based devices were mounted onto custom PCBs and wire-bonded. Electrical signals were routed to the chip through a custom circuit consisting of a microcontroller, shift registers, and relays. Electrode configurations were assigned through software (MATLAB R2022a) commands sent via USB connection to the microcontroller. Samples were imaged at 4× or 10× magnification through appropriate fluorescence bandpass filters using a Nikon Eclipse 50i microscope affixed to a mercury lamp or an X-Cite Xylis LED excitation source. Panels depicting fluorescence images in FIG. 80 consist of still frames from videos acquired during experimentation. All images were post-processed with brightness and contrast enhancement, false coloring, and annotation. Regarding annotation, arrows indicate the direction of DNA motion. Boxes with solid lines indicate activated (oscillatory, or high potential) nodes. Dashed lines indicate inactive (high impedance) nodes. Electrodes otherwise lacking annotation are grounded.

FIG. 80 depicts electrokinetic manipulation of PhiX DNA and small oligonucleotides on silicon-based devices. Panel A shows planar passivated nodes in a hexagonal array at the Device 1 surface. Panel B depicts planar metal nodes in a square array at the Device 2 surface. Panels C through E demonstrate frequency-dependent localization and ejection of PhiX DNA from an aqueous suspension at Device 1 nodes. Tuning the frequency of the applied AC signal shifts the localization pattern of PhiX DNA within the electrode plane (panels c and d). At 50 kHz, PhiX DNA ejects from the site of localization at the active electrode (panel e). These distinct localization patterns enable sophisticated control of chip-based electrokinetic operations, which may be used to position the DNA payload within a droplet or droplet generator. Panels F through H depict one such fundamental operation as the electrophoretic transfer of small oligonucleotides between discrete nodes on Device 1 through execution of a temporal sequence of electrode configurations. Panel F shows the localization of small oligonucleotides at an active node set to 2.5 V. In panel G, an adjacent node is activated while the initial localization site is floated. Oligonucleotides respond to the change in the electric field pattern by transporting from the initial localization site to the active node. Grounding the initial localization site alters the pattern of the electric field once more, completing the transfer of oligonucleotides (as seen in Panel H).

### Experimental Demonstrations of EWOD Methods for Cell Packaging and Functional Assays

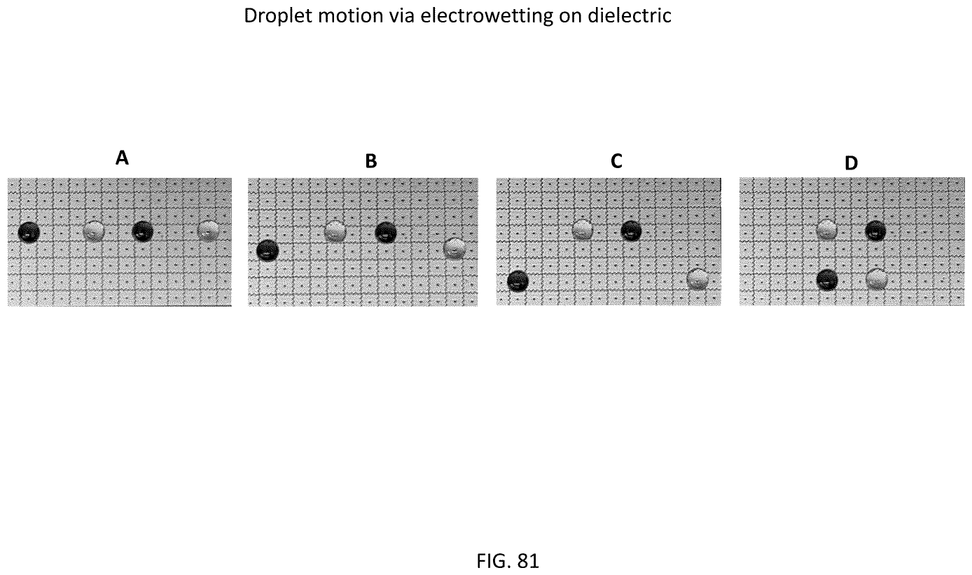

FIG. 81 shows droplet motion via electrowetting on dielectric. Image A illustrates four droplets positioned on electrode array. Image B illustrates left and rightmost droplets move one electrode down by applying 240V. Image C illustrates left and rightmost droplets move two electrodes down by sequentially applying 240V. Image D illustrates leftmost droplet (blue) moves four electrodes to the right and rightmost droplet (yellow) moves four electrodes to the left by sequentially applying 240V.

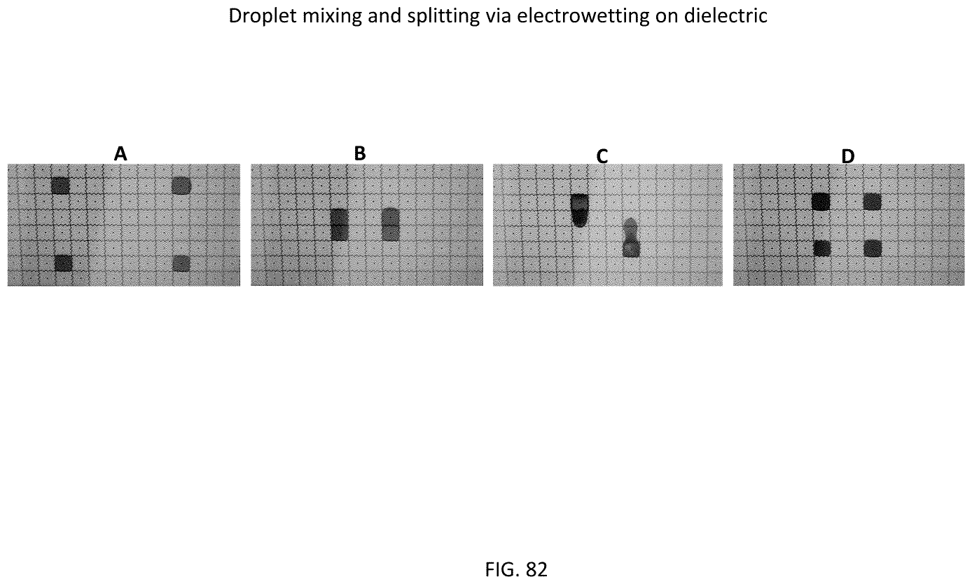

FIG. 82 shows droplet mixing and splitting via electrowetting on dielectric. Image A illustrates red and blue droplets dispensed onto electrode array. Image B illustrates droplets brought into proximity then merged by applying 240V to adjacent electrodes. Image C illustrates droplets are mixed by moving to and from adjacent electrode by applying 240V. Image D illustrates homogenous dark red mixture produced by droplet mixing and splitting.

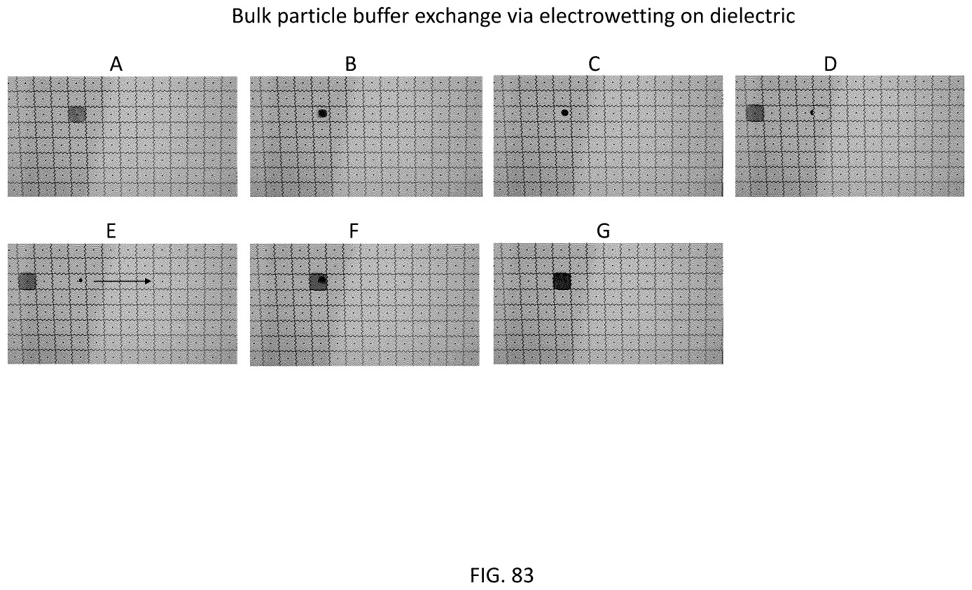

FIG. 83 shows bulk particle buffer exchange via electrowetting on dielectric. Image A illustrates a droplet containing magnetic beads on electrode array. Image B illustrates a magnet engaged; beads beginning to aggregate. Image C illustrates beads fully aggregated within original droplet. Image D illustrates the initial droplet moved to an adjacent electrode by sequentially applying 240V. Beads remain fixed by magnet. New droplet containing blue buffer dispensed onto electrode array. Image E illustrates the initial droplet moved five electrodes to the right. Beads remain fixed on electrode. Image F illustrates a blue droplet positioned over bead aggregate. Magnet is disengaged. Image G illustrates beads are released from electrode and resuspended in blue droplet.

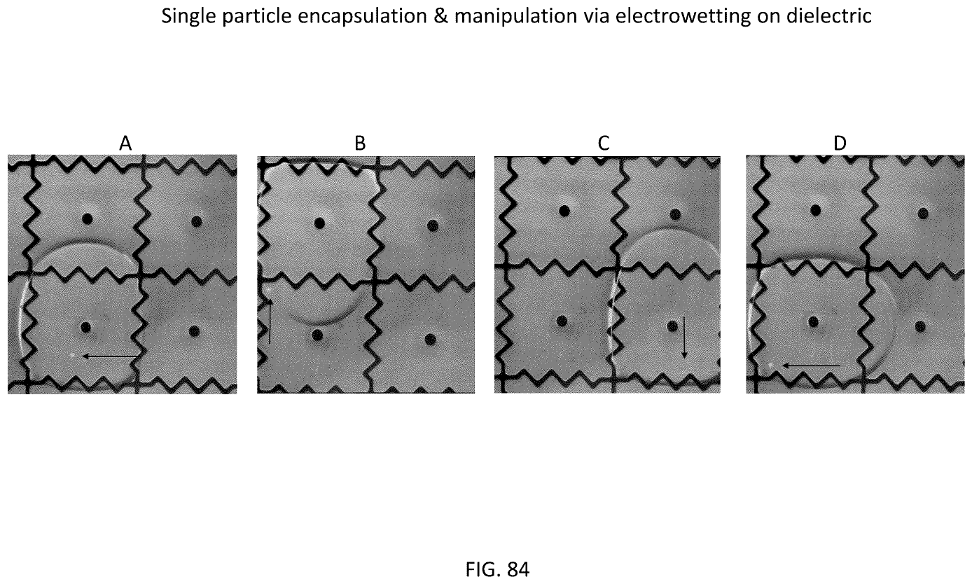

FIG. 84 shows single particle encapsulation & manipulation via electrowetting on dielectric. Image A illustrates a droplet containing single 100 μm bead positioned on an electrode array. Image B illustrates the bead remains inside the droplet as it moves to the electrode above by applying 240V. Image C illustrates the bead remains inside the droplet as it moves one electrode to the right and then one electrode down by sequentially applying 240V. Image D illustrates the droplet containing single 100 μm bead moves back to starting electrode by applying 240V to the adjacent electrode.

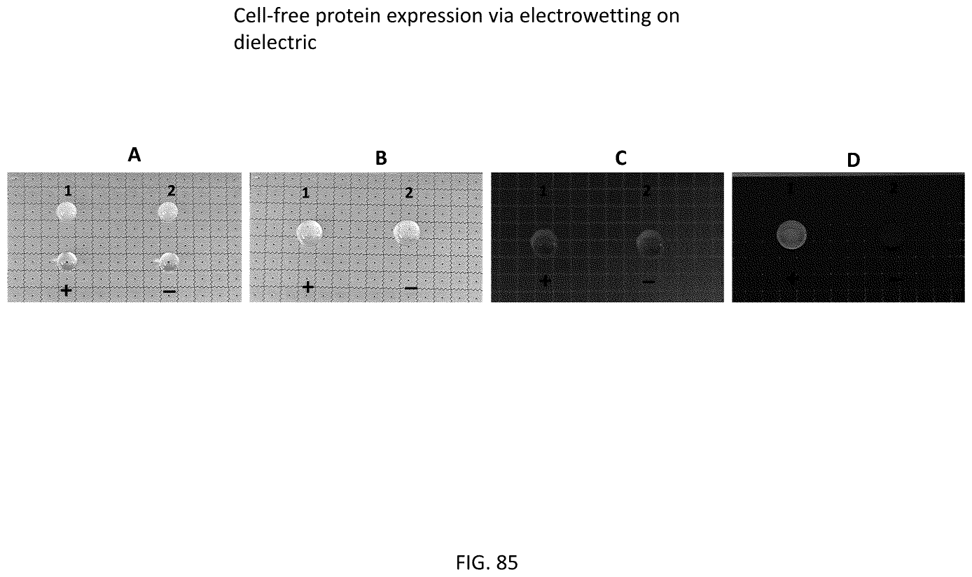

FIG. 85 shows cell-free protein expression via electrowetting on dielectric. A droplet containing cell lysate is mixed with a droplet containing 1 μM GFP/Kan^{r} plasmid DNA on an EWOD device. The merged cell lysate/DNA droplet is mixed by moving the droplet up and down the electrode array by applying 240V DC voltage to an adjacent electrode. The cell lysate/DNA droplet is then incubated at 37 C until green fluorescent protein is observed. Green fluorescent protein produced via cell-free expression is observable by eye after one hour incubation.

Image A illustrates droplets ‘1’ & ‘2’, which are droplets containing cell lysate. Image A also illustrates a ‘+’ droplet containing 1 μM GFP/Kan^{r} plasmid DNA. Image A also illustrates a ‘−’ droplet containing water only. Image B illustrates droplets 1 & 2 merged with + or − droplet by applying 240V to adjacent electrodes. Image C illustrates time=0 CFE reaction. Image D illustrates time=1 hour reaction; Droplet 1 green fluorescent protein expression from plasmid DNA.

A droplet containing cell lysate is mixed with a droplet containing 1 μM GFP/Kan^{r} plasmid DNA on an EWOD device. The merged cell lysate/DNA droplet is mixed by moving the droplet up and down the electrode array by applying 240V DC voltage to an adjacent electrode. The cell lysate/DNA droplet is then incubated at 37 C until green fluorescent protein is observed. Green fluorescent protein produced via cell-free expression is observable by eye after one hour incubation.

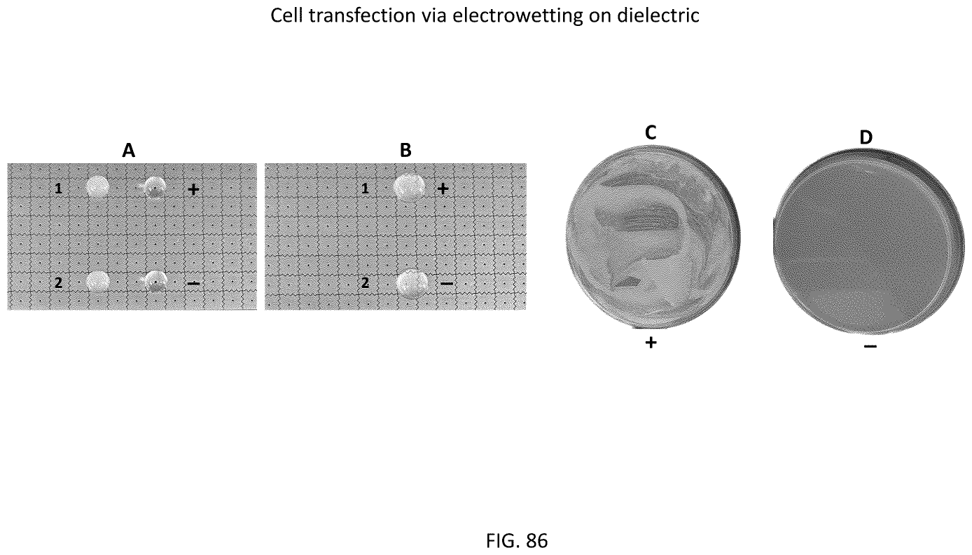

FIG. 86 shows cell transformation via electrowetting on dielectric. A droplet containing competent E. coli cells is merged with a droplet containing 1 μM GFP/Kan^{r} plasmid DNA on an EWOD device by applying 240V DC voltage to an adjacent electrode. The merged droplet incubates at room temperature on the EWOD device for 15 minutes. The droplet is then removed from the EWOD device, plated on Kan^{r} selective media and incubated overnight at 37 C. A lawn of transfected E. coli cells is observed after the overnight incubation, indicating the E. coli cells were successfully transfected with the GFP/Kan^{r} plasmid DNA.

Image A illustrates droplets ‘1’ & ‘2’, which are droplets containing competent E. coli cells. Image A also illustrates a ‘+’ droplet containing 1 μM GFP/Kan^{r} plasmid DNA. Image A also illustrates a ‘−’: droplet containing water only. Image B illustrates droplets 1 & 2 merged with + or − droplet by applying 240V to adjacent electrodes. Image C illustrates transformation reaction from Droplet ‘1/+’ plated on selective media after overnight incubation. Image D illustrates transformation reaction from Droplet ‘2/−’ plated on selective media after overnight incubation.

A droplet containing competent E. coli cells is merged with a droplet containing 1 μM GFP/Kan^{r} plasmid DNA on an EWOD device by applying 240V DC voltage to an adjacent electrode. The merged droplet incubates at room temperature on the EWOD device for 15 minutes. The droplet is then removed from the EWOD device, plated on Kan^{r} selective media and incubated overnight at 37 C. A lawn of transfected E. coli cells is observed after the overnight incubation, indicating the E. coli cells were successfully transfected with the GFP/Kan^{r} plasmid DNA.

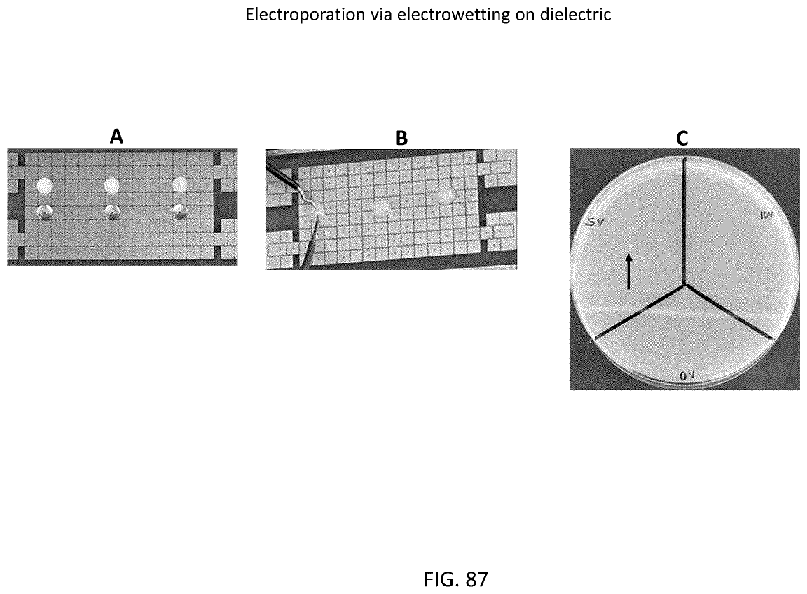

FIG. 87 shows electroporation via electrowetting on dielectric. Three droplets containing electrically competent E. coli cells are each merged with a droplet containing 150 ng GFP/Kan^{r} plasmid DNA on an EWOD device by applying 240V to adjacent electrodes. Each merged droplet experiences either 0V, 5V or 10V DC voltage delivered via PalmSens for 5 seconds. Droplets are then removed from the EWOD device, plated on Kan^{r} selective media and incubated overnight at 37 C. A single colony is observed for the 5V electroporation condition after the overnight incubation, indicating a small number of E. coli cells were successfully transfected with the GFP/Kan^{r} plasmid DNA via electroporation technique.

Image A illustrates droplets containing electrically competent E. coli cells (Top) to be merged with droplets containing GFP/Kanr plasmid DNA (bottom) by applying 240V to adjacent electrodes. Image B illustrates, from left to right: Droplets exposed to 0, 5 or 10V DC for 5 seconds. Image C illustrates electroporation reactions plated on selective media.

Three droplets containing electrically competent E. coli cells are each merged with a droplet containing 150 ng GFP/Kan^{r} plasmid DNA on an EWOD device by applying 240V to adjacent electrodes. Each merged droplet experiences either 0V, 5V or 10V DC voltage delivered via PalmSens for 5 seconds. Droplets are then removed from the EWOD device, plated on Kan^{r} selective media and incubated overnight at 37 C. A single colony is observed for the 5V electroporation condition after the overnight incubation, indicating a small number of E. coli cells were successfully transfected with the GFP/Kan^{r} plasmid DNA via electroporation technique.

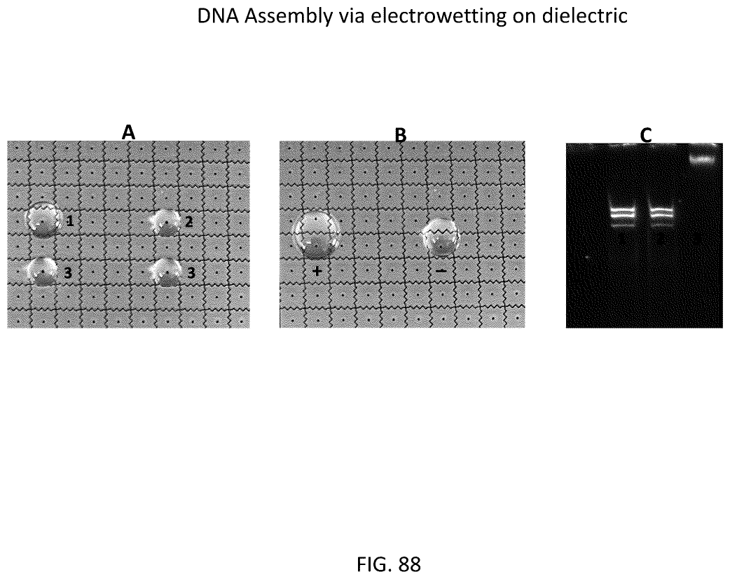

FIG. 88 shows DNA assembly via electrowetting on dielectric. A droplet containing 2× DNA assembly master mix is merged with a droplet of equal volume containing fragmented reference plasmid DNA by applying 240V to an adjacent electrode. The merged droplet is incubated at 50 C for one hour. The droplet is then removed from the EWOD device and subjected to gel electrophoresis. The unassembled fragmented reference plasmid is observed as three distinct bands and the assembled product is observed as a single band on the stained gel.

Image A illustrates two droplets of Droplet 1, which comprises Assembly master mix. Image A also illustrates Droplet 2, which comprises Deionized H2O. Image A also illustrates Droplet 3, which comprises fragmented reference plasmid DNA. Image B illustrates a “+” droplet, which comprises Assembly master mix merged with fragmented reference plasmid DNA; positive assembly reaction. Image B also illustrates a “−” droplet, which comprises deionized H₂O droplet merged with fragmented reference plasmid DNA; negative assembly reaction. Image C illustrates gel electrophoresis 1: Fragmented reference plasmid DNA. Image C also illustrates gel electrophoresis 2: Negative assembly reaction. Image C also illustrates gel electrophoresis 3: Positive assembly reaction.

A droplet containing 2× DNA assembly master mix is merged with a droplet of equal volume containing fragmented reference plasmid DNA by applying 240V to an adjacent electrode. The merged droplet is incubated at 50 C for one hour. The droplet is then removed from the EWOD device and subjected to gel electrophoresis. The unassembled fragmented reference plasmid is observed as three distinct bands and the assembled product is observed as a single band on the stained gel.

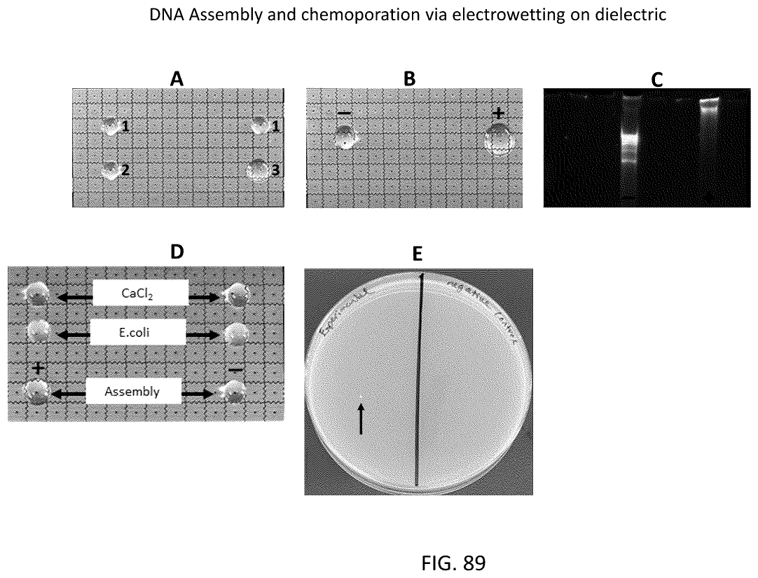

FIG. 89 shows DNA assembly and chemoporation via electrowetting on dielectric. A droplet containing 2× DNA assembly master mix is merged with a droplet of equal volume containing 150 ng fragmented GFP/Kan^{r} plasmid DNA by applying 240V to an adjacent electrode. The merged droplet is incubated at 50 C for one hour. After incubation, a droplet containing 400 mM CaCl₂ and a droplet containing competent E. coli cells are dispensed on the EWOD device. The CaCl₂ droplet is first merged with the competent E. coli cells droplet. The + or − assembly droplets are then merged with the CaCl₂/cell droplet to chemically transfect the E. coli cells with the assembled GFP/Kan^{r} plasmid DNA. After a 15-minute incubation at room temperature, the droplets are plated on Kan^{r} selective media and incubated overnight at 37 C. A single colony is produced and shows that at least one cell was chemically transfected on the EWOD device.

Image A illustrates two droplets that are identified as a “1” droplet. A “1” droplet comprises fragmented GFP/Kan^{r} plasmid DNA. Image A also illustrates a “2” droplet, which comprises deionized H₂O. Image A also illustrates a “3” droplet, which comprises an assembly master mix. Image B illustrates a “−” droplet. A “−” droplet comprises droplets 1 & 2 from Image A that have been merged by applying 240V to an adjacent electrode. The use of “−” denotes negative control for assembly reaction. Image B also illustrates a “+” droplet, which comprises droplets 1 & 3 from Image A that have been merged by applying 240V to adjacent electrode. The use of “+” denotes positive sample assembly reaction. Image C illustrates gel electrophoresis on assembly reactions from Image B. Image D illustrates droplets containing 400 mM CaCl₂ (top) merged with E. coli cell droplets (middle) by applying 240V to adjacent electrodes. In Image D, CaCl₂/E. coli droplets merged with droplets containing assembly reactions from Image B by applying 240V to adjacent electrodes. The use of “+” and “−” denotes positive or negative reactions from Image B. Image E illustrates GFP/Kan^{r} assembled plasmid DNA-transformation reactions plated on selective media.

A droplet containing 2× DNA assembly master mix is merged with a droplet of equal volume containing 150 ng fragmented GFP/Kan^{r} plasmid DNA by applying 240V to an adjacent electrode. The merged droplet is incubated at 50 C for one hour. After incubation, a droplet containing 400 mM CaCl₂ and a droplet containing competent E. coli cells are dispensed on the EWOD device. The CaCl₂ droplet is first merged with the competent E. coli cells droplet. The + or − assembly droplets are then merged with the CaCl₂/cell droplet to chemically transfect the E. coli cells with the assembled GFP/Kan^{r} plasmid DNA. After a 15-minute incubation at room temperature, the droplets are plated on Kan^{r} selective media and incubated overnight at 37 C. A single colony is produced and shows that at least one cell was chemically transfected on the EWOD device.

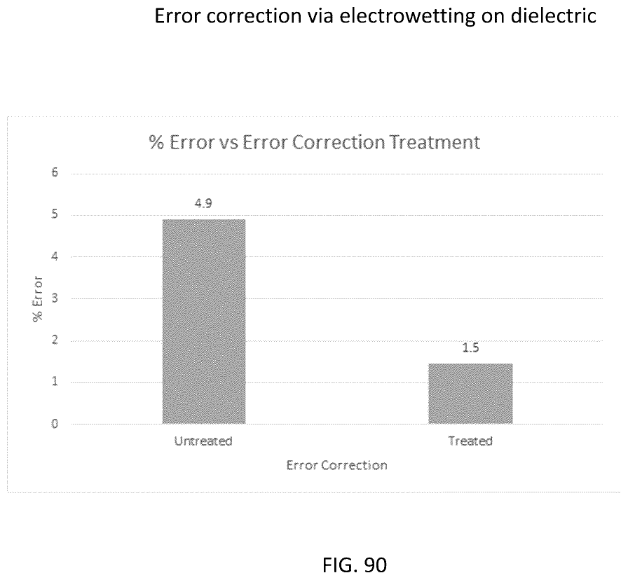

FIG. 90 shows error correction via electrowetting on dielectric. A droplet containing error correction enzymes is merged with a droplet containing a library in which 5% of the oligonucleotides contain errors. The merged droplet is incubated at room temperature for one hour and is then removed from the EWOD device. The droplet then undergoes library preparation and DNA sequencing to determine the percentage of errors present in the untreated vs treated library pools. The percentage of errors in the untreated library was measured as 4.9%. The percentage of errors in the treated library pool was measured as 1.5%, roughly a 3× decrease in errors relative to the untreated library.

### Additional Aspects and Embodiments

Additional aspects and embodiments are provided herein. For example, an initial aspect provides an Electro-Wetting On Dielectric array device comprising cells in droplets. Another aspect provides an Electro-Wetting On Dielectric array device comprising single cells in droplets. A further aspect provides an Electro-Wetting On Dielectric array device comprising multiple cells in droplets.

Another aspect provides an Electro-Wetting On Dielectric array device comprising DNA payloads in droplets. An additional aspect provides an Electro-Wetting On Dielectric array device comprising single DNA molecules in droplets. A further aspect provides an Electro-Wetting On Dielectric array device comprising multiple DNA molecules in droplets. In some embodiments, an Electro-Wetting On Dielectric array device comprises single DNA molecules that are oligos (e.g., less than 100 bases). In some embodiments, an Electro-Wetting On Dielectric array device comprises multiple DNA molecules that are oligos (e.g., less than 100 bases). In some embodiments, an Electro-Wetting On Dielectric array device comprises single DNA molecules that are in the range of 0.1 kilo-bases to 1 kb. In some embodiments, an Electro-Wetting On Dielectric array device comprises multiple DNA molecules that are in the range of 0.1 kilo-bases to 1 kb. In some embodiments, an Electro-Wetting On Dielectric array device comprises single DNA molecules that are in the range of 1 kb-10 kb. In some embodiments, an Electro-Wetting On Dielectric array device comprises multiple DNA molecules that are in the range of 1 kb-10 kb. In some embodiments, an Electro-Wetting On Dielectric array device comprises single DNA molecules that are in the range of 10 kb-100 kb. In some embodiments, an Electro-Wetting On Dielectric array device comprises multiple DNA molecules that are in the range of 10 kb-100 kb. In some embodiments, an Electro-Wetting On Dielectric array device comprises single DNA molecules that are in the range of 1 kb-1 Mega-bases. In some embodiments, an Electro-Wetting On Dielectric array device comprises multiple DNA molecules that are in the range of 1 kb-1 Mega-bases. In some embodiments, an Electro-Wetting On Dielectric array device comprises single DNA molecules that are more than 1 Mb. In some embodiments, an Electro-Wetting On Dielectric array device comprises multiple DNA molecules that are more than 1 Mb. In some embodiments, DNA molecules may be bare DNA. In some embodiments, DNA molecules may be DNA complexed with other factors. In some embodiments, DNA molecules may be complexed with factors that organize or structure DNA. In some embodiments, DNA molecules may be complexed with histones.

An alternative aspect provides an Electro-Wetting On Dielectric array device comprising cells in droplets with additional sensors and/or actuators. Another aspect provides an Electro-Wetting On Dielectric array device comprising single cells in droplets with additional sensors and/or actuators. A further aspect provides an Electro-Wetting On Dielectric array device comprising multiple cells in droplets with additional sensors and/or actuators.

Additionally, embodiments may be provided of methods for delivering DNA payloads into cell packages. In some embodiments, methods of delivering DNA payloads into cell packages may comprise presenting the DNA payloads in droplets on an Electro-Wetting On Dielectric array device. Additionally, in some embodiments droplets on the Electro-Wetting On Dielectric array device comprise cell packages. Further, the Electro-Wetting On Dielectric array device may be used to merge droplets. Within the merged droplets, cell packages and payloads may merge into the same droplets. Once the cell packages and payloads are brought into the same droplet, an insertion method may take place. An insertion method may deliver the DNA into the cells. In some embodiments, an insertion method may comprise an insertion reaction. In some embodiments, methods of delivering DNA payloads into cell packages may occur on an array so as to allow substantially parallel processes. In some embodiments, methods of delivering DNA payloads into cell packages may occur on an array having at least 100 processes in parallel, at least 1000 processes in parallel, at least 10,000 processes in parallel, at least 100,000 processes in parallel, at least 1 million processes in parallel, at least 10 million processes in parallel, or more than 10 million processes in parallel. In some embodiments, the array or arrays on which these processes occur may have up to 1,000 electrodes, up to 10,000 electrodes, up to 100,000 electrodes, up to 1 million electrodes, up to 10 million electrodes, or more.

In some embodiments, an insertion method comprises electroporation performed on an Electro-Wetting On Dielectric array device. In particular, an insertion method may comprise single-cell electroporation. In some embodiments, an insertion method comprises vesicle merger performed on an Electro-Wetting On Dielectric array device. In particular, an insertion method may comprise voltage-enhanced vesicle merger. In some embodiments, an insertion method comprises viral transduction performed on an Electro-Wetting On Dielectric array device.

In some embodiments, an insertion method comprises a micro-injection performed on an Electro-Wetting On Dielectric array device. In some embodiments, the micro-injection is performed using injector devices. In some embodiments, the micro-injection is performed using direct injector devices. In some embodiments, the micro-injection is performed optionally comprising a positioner device. In some embodiments, an insertion method comprises a nano-injection performed on an Electro-Wetting On Dielectric array device. In some embodiments, the nano-injection is performed using injector devices. In some embodiments, the nano-injection is performed using direct injector devices. In some embodiments, the nano-injection is performed optionally comprising a positioner device. In some embodiments, an injector device may have an internal diameter of 1 nanometer, 10 nanometers, 100 nanometers, one micron, five microns, or more than five microns. In some embodiments, an injector device may have an internal diameter of about 10 nanometers, about 20 nanometers, about 30 nanometers, about 40 nanometers, about 50 nanometers, about 60 nanometers, about 70 nanometers, about 80 nanometers, about 90 nanometers, about 100 nanometers, or about 110 nanometers. In some embodiments, an injector device may have an external diameter of 10 nanometers, 100 nanometers, 1 micron, 5 microns, 10 microns, or more than 10 microns. In some embodiments, an internal diameter may be a channel. In some embodiments, an internal diameter may be a tube. A positioner device may be used to precisely position a cell for injection. In some embodiments, an insertion method comprises a method of transduction performed on an Electro-Wetting On Dielectric array device. In some embodiments, an insertion method comprises a method of transfection performed on an Electro-Wetting On Dielectric array device. In some embodiments, an insertion method comprises chemo-poration performed on an Electro-Wetting On Dielectric array device. In methods using chemo-poration, methods of chemically induced transfection may be used as insertion methods.

In some embodiments, an insertion method may be performed on an Electro-Wetting On Dielectric array device that is cell-free or essentially cell-free. For example, in some embodiments, an insertion method may be performed upon a droplet that is within an Electro-Wetting On Dielectric array device. In some aspects of these embodiments, the droplet may comprise reagents for cell-free protein expression. Further, in some aspects of these embodiments where an insertion method is performed upon a droplet that is within an Electro-Wetting On Dielectric array device, insertion may be achieved by merging the droplet having reagents for cell-free expression with a DNA payload droplet. Additionally, in some aspects of these embodiments where an insertion method is performed upon a droplet that is within an Electro-Wetting On Dielectric array device, insertion may be achieved by merging the droplet having reagents for cell-free expression with a RNA payload droplet.

In some embodiments, cell and payload droplets are presented in a patter so as to enable massively parallel payload delivery. In some embodiments where the cell and payload droplets are presented in a pattern to efficiently so as to enable the cells to be loaded with multiple DNA payloads, from a selection of payloads options, in a massively parallel fashion.

In some embodiments, a method is provided for assessing the results of DNA payload delivery. In some embodiments, DNA payload delivery may be assessed through subsequent stimulation. In some embodiments, DNA payload delivery may be assessed through subsequent actuation. In some embodiments, DNA payload delivery may be assessed through sensing directly on the EWOD array. In some embodiments, DNA payload delivery may be assessed though the use of off-array optical sensing. In some embodiments, DNA payload delivery may be assessed once the DNA payload has been inserted into a cell. In some embodiments, DNA payload delivery may be assessed once the DNA payload has been inserted into a cell-free environment. In some embodiments, DNA payload delivery may be assessed once the DNA payload has been inserted into a cell-free droplet.

In some embodiments, a method is provided for assessing the results of RNA payload delivery. In some embodiments, RNA payload delivery may be assessed through subsequent stimulation. In some embodiments, RNA payload delivery may be assessed through subsequent actuation. In some embodiments, RNA payload delivery may be assessed through sensing directly on the EWOD array. In some embodiments, DNA payload delivery may be assessed though the use of off-array optical sensing. In some embodiments, DNA payload delivery may be assessed once the RNA payload has been inserted into a cell. In some embodiments, RNA payload delivery may be assessed once the RNA payload has been inserted into a cell-free environment. In some embodiments, RNA payload delivery may be assessed once the RNA payload has been inserted into a cell-free droplet.

In some embodiments, an EWOD array may be organized so as to comprise a droplet QC Zone, a Treatment Zone, an Actuator Zone, and/or a Sensor Zone.

In some embodiments, a method is provided of using EWOD array devices to deliver a DNA payload into a cell (e.g. by chemo-poration or electro-portion or direct injection), and performing functional screening of the results by using a fluorescent reporter assay and detecting the signal either with on device photo-sensors, or an off-device optical system, and where the payload DNA transfer may be of a payload which is a single gene, a gene set within one long DNA molecule, an artificial chromosome within one DNA molecule, or the payload may comprise collection of multiple DNA molecules such as guide RNA's for CRISPR genome editing, or the oligo set for the MAGE process of genome engineering.

In some embodiments, methods are provided for using EWOD array devises to carry out specific workflows of interest, such as providing payload delivery to cells and/or functional screening of the resulting cells. In some embodiments, a system is provided that comprises an EWOD array chip device that is integrated with a nanochannel chip device. In some embodiments, a system is provided that comprises an EWOD array chip device integrated with a DNA oligonucleotide synthesis and assembly chip device. In some embodiments, an EWOD array chip device such as those provided herein may be used to provide for the DNA payloads for the payload droplets on an array.

In some embodiments, a system comprises an EWOD array chip device integrated with a nanochannel array and a DNA oligonucleotide synthesis and assembly array chip device. With this system, DNA payload materials may be transferred from one device to another. In some embodiments, a system for cellular engineering is provided in which the fluidic integration of multiple chip devices may comprise the use of an EWOD interface device that may be used to perform fluid transfers between chip devices. In some embodiments, a TFT EWOD interface device may be used to enable the movement of fluids across multiple chips.

In some embodiments, a system for cellular engineering is provided in which the fluid integration of multiple chip devices comprises the use of an EWOD bridge connector device. The EWOD bridge connector device may be used to enable fluid transfers between chip devices.

In some embodiments of systems as discussed herein, the fluidic integration may comprise the use of an EWOD interface device. In some embodiments, the EWOD interface device may be used to transfer fluids between chip devices. In some embodiments of systems as discussed herein, the fluidic integration may comprise the use of a TFT EWOD interface device. In some embodiments, the TFT EWOD interface device may be used to transfer fluids between chip devices. In some embodiments of systems as discussed herein, the fluidic integration may comprise the use of an EWOD bridge connector device. In some embodiments, the EWOD bridge connector device may be used to transfer fluids between chip devices. In some embodiments, the EWOD transfer may use a TFT transfer EWOD device.

In some embodiments, an EWOD array device as disclosed herein may be implemented as a CMOS chip device. In some embodiments, the use of high pixel density CMOS chips for EWOD may have a pixel density of 10,000 pixels per square millimeter. In some embodiments, the use of high pixel density CMOS chips for EWOD may have a pixel density of 100,000 pixels per square millimeter. In some embodiments, the use of high pixel density CMOS chips for EWOD may have a pixel density of 1,000,000 pixels per square millimeter.

In some embodiments, CMOS EMOD array devices may be used to perform payload delivery into cells. In some embodiments, CMOS EMOD array devices may be used to perform cell functional screening.

In some embodiments, methods are provided for using CMOS EWOD devices to achieve massively parallel controlled workflows. In some embodiments, methods are provided for using CMOS EWOD devices to achieve scaling to massive, high-density arrays. In some embodiments, methods are provided for using CMOS EWOD devices to perform the “MAGE” method as discussed in the definitions section. In particular, in some embodiments, methods are provided for using CMOS EWOD devices for to perform the “MAGE” method of genome editing on cells in a massively parallel and scalable fashion.

In some embodiments, a system is provided for performing cell packaging and functional screening on a CMOS chip EWOD device, with features as indicated in FIG. 70.

In some embodiments, CMOS EWOD devices as describe herein may have more than 10,000 wetting electrodes. In some embodiments, CMOS EWOD devices as describe herein may have more than 100,000 wetting electrodes. In some embodiments, CMOS EWOD devices as describe herein may have more than 1,000,000 wetting electrodes. In some embodiments, CMOS EWOD devices as describe herein may have an electrode array density of at least 1,000 per square millimeter. In some embodiments, CMOS EWOD devices as describe herein may have an electrode array density of at least 10,000 per square millimeter. In some embodiments, CMOS EWOD devices as describe herein may have an electrode array density of at least 100,000 per square millimeter. In some embodiments, CMOS EWOD devices as describe herein may have an electrode array density of at least 1,000,000 per square millimeter. In some embodiments, CMOS EWOD devices as describe herein may have an electrode array density of at least 10,000,000 per square millimeter.

In some embodiments, methods are provided for the performance of specific workflows of systems described herein. In some embodiments, methods are provided for the specific “MAGE” workflow. In some embodiments, methods are provided for delivering cells and DNA payloads onto CMOS EWOD arrays, such as for subsequent processing. In some embodiments, devices are provided for delivering cells and DNA payloads onto CMOS EWOD arrays, such as for subsequent processing. In some embodiments, an EWOD array device as discussed herein may also be a CMOS chip device. In some embodiments, an EWOD system as indicated in FIG. 70 is provided, wherein the cell holding module indicated comprises an EWOD array device with temperature actuators. In some embodiments, this EWOD array device as provided in FIG. 70 with temperature actuators may be used for methods in which cells are incubated and grown within the holding module.

In some embodiments, a complete system is provided for cellular engineering comprising multiple chip devices that are able to perform DNA synthesis, assembly, cell-packaging, and functional testing. In some embodiments of this complete system for cellular engineering, the multiple chip devices may comprise one or more EWOD devices. In some embodiments of this complete system for cellular engineering, the multiple chip devices may comprise one or more chip devices. In some embodiments of this complete system for cellular engineering, the multiple chip devices may comprise one or more EWOD interface devices. In some embodiments of this complete system for cellular engineering, the multiple chip devices may comprise one or more eternal optical/imaging systems. In some embodiments of this complete system for cellular engineering, the multiple chip devices may comprise EWOD chip devices that perform elements of DNA assembly and/or DNA error correction. In some embodiments, an EWOD droplet device as discussed herein may be used to perform one or more aspects of DNA assembly and/or DNA error correction procedures.

### Definitions and Interpretations

As used herein, the term “Electrowetting” means the use of voltage or electrical influence to cause droplets of solution to move on a surface.

As used herein, the term “Aqueous Droplet” refers to a liquid droplet composed of H₂O as the solvent, or H₂O is a substantial component of the total liquid content of the droplet. Such droplets may contain solutions that are standard biochemical buffers, for example, or other solutions commonly used in molecular biology methods. Such droplets may also contain contents beyond these solutions, such as cells, or DNA, or other molecules or elements.

As used herein, the term “oil” refers to any of the oils commonly used as the ambient liquid phase for EWOD devices, or most generally, to other fluids that are immiscible with water that are used for this purpose.

As used herein, the term “Fluid” may refer to a liquid or a gas, as is common in the fields of Fluid Dynamics and Materials Science, or may refer to any other material that may be used as the ambient phase in an EWOD device.

As used herein, the term “Wetting Electrode” refers to the electrode that is used to drive motion in an EWOD device. Such electrodes may be metal, semiconductors, doped semiconductors, or any other material capable of acting as an electrode for the purposes of electrically including droplets in an EWOD device.

As used herein, the term “Dielectric” refers to any material layer that can perform the functional role of an electrically insulating layer protecting the electrode for an EWOD device. Such dielectric layers may be glasses, plastics, polymers, ceramics, or semiconductor oxides that are insulators, or may be functional coatings on the electrodes by materials that are insulating, such as monolayer molecular coatings that are insulating.

As used herein, the term “Hydrophobic Surface” refers to the thin film in an EWOD device that is in direct contact with the aqueous droplets, and fulfills the functional role of preventing the aqueous droplets of completely wetting the surface, and preferably causes such droplets to have hydrophobic contact angle when at equilibrium.

For purposes herein, term “EWOD”, which is an acronym for Electro-Wetting On Dielectric, refers both specially to the EWOD process of droplet control, and, where the context allows, refers more broadly to any droplet control mechanisms that rely on electrical forces that can drive droplets on an array of electrodes, such as where the electrical fields produce an electrically induced change in droplet surface wetting, or in the attraction or repulsion of the droplet from a surface, including such phenomena as electro-wetting and electro-dewetting. In this broader sense as used herein, the term “EWOD” is meant to be synonymous with and encompassing of Digital Micro-Fluidic (DMF) technologies.

As used herein, the term “EWOD electrode” refers to any electrode that is used to drive droplet motions through electrowetting and related effects. In context, where this makes sense, just the term “electrode” can be taken as referring to the “EWOD electrode”. This term, when it makes sense, may be taken to refer to the electrode in an electrowetting device even when it does have a dielectric layer. In this context, it is to be understood that the electrowetting effects used to drive droplet motion include the phenomena of “electro-dewetting”, in which the droplets include surfactants that produce a surface charge, which can be repelled by a like charge being placed on the driving electrode, to “de-wett” the droplet from the electrode surface.

As used herein, the term “sensor” refers to any sensing device that can be implemented as an all-electronic or partially electronic sensor.

As used herein, the term “actuator” refers to any actuating device that can be implemented as an all-electronic or partially electronic actuator.

As used herein, the term “DNA Payload” refers to DNA that is intended to be delivered into a cellular package. This may be a single molecule of DNA, or multiple molecules of DNA. In contexts where this makes sense, it may also be an RNA molecule, or other nucleic acids or related molecules as further outlined below.

As used herein, the term “Cell” is broadly meant to be a functional container that can contain the DNA payload, and other required reactants, for the subsequent functional assessments of the resulting cell containing the payload. This cell may be a biological cell, such as a bacterial cell, a fungal cell, an animal cell, or a prokaryotic or eukaryotic cell, or a viral particle, or cells derived from these, such as just the cell having biological components removed, such as having the genomic DNA removed. Or such cells reduced to simple containers, for the packaging of the payload DNA, such as consists of just the cell wall and a subset of the usual internal cytoplasmic contents relative to a biological cell. In context where this makes sense, cell may include a vesicle, such as a lipid vesicle, or a viral particle. In the context of cell-free expression, cell may be taken to refer to the liquid droplet itself, which plays the confinement role of the cell for the reagents in the reactions. In the broadest context, cell refers to anything that provides the containment of the payload DNA, plus the other required reagents, for performing the functions that are the subject of the later functional assays.

As used herein, the term “Transduction” refers to methods of delivering a DNA payload to a cell that rely on a virus to perform the transfer or a portion of the transfer.

As used herein, the term “Transfection” refers to any or all of the many methods by which DNA is transported into a cell from an external location, including, for example, chemically mediated transport, vesicle or cell fusions, or mechanical or electrically mediated methods.

As used herein, the term “Chip” refers to a semiconductor integrated circuit chip, or such a chip with additional post-processing to add surface features, such as the electrodes or material layers as required to fashion EWOD devices, or such a chip mounted into packaging materials, such as being wire-bonded to a chip carrier. In certain contexts where this is clear, “Chip” may refer more specifically to CMOS integrated circuit chips

As used herein, the term “CMOS”, which is an acronym for “Complementary Metal Oxide Semiconductor”, and refers to chips that are made by the CMOS process.

As used herein, the term “CMOS device” or “CMOS EWOD device” refers to a device that is embodied as a CMOS chip, or as a CMOS chip with additional postprocessing to add auxiliary features, such as surface coatings or thin films, and surface electrodes, or such electrodes embedded in such coating or thin film layers.

As used herein, the term “TFT”, which is an acronym for “Thin Film Transistor”, refers to a transistor made using thin-film transistor technology and processes. The term “TFT” may also in context refer to the processes used to make TFT's and TFT-containing devices, which encompass a distinct class of manufacturing processes and methods for making transistors and related circuits on diverse insulating substrates, including fabrication on transparent glass substrates, and optionally using Transparent Conducting Electrode (TCE) materials such as Indium Tin Oxide (ITO), Indium Zinc Oxide (IZO), Aluminum Zinc Oxide (IZO), and other such transparent electrode materials, such that devices made with TFT circuits and TCE technologies may be substantially transparent. Such transparent TFT devices fabricated on glass or plastic sheets, are widely used for display screens for computers, televisions, and cell phones. As is made clear by context, the term “TFT” in a given context may therefore refer to such transistors, or circuits comprising such transistors, or devices that comprise such circuits, or devices that are made using the manufacturing techniques, processes and materials associated with manufacturing of TFTs. For example, reference to a “TFT display” on a cell phone broadly encompasses all these meanings.

As used herein, the term “TFT array” or “TFT active matrix array” refers to devices which comprise an array of pixels, often a rectangular array, but any other regular arrays such as triangular or hexagonal arrays are also included, in which pixels in the array comprises one or more TFT's.

As used herein, the term “TFT device” or “TFT EWOD device”, or “EWOD TFT device” refer to devices in which a TFT active matrix array is used to control the array of activating electrodes used to drive droplet motion in an EWOD device.

As used herein, the term “interface device” or “EWOD interface device” refers to any microfluidic electrowetting array device with the capability to transfer physical material from one device to another.

As used herein, the acronym “PCB” refers to a Printed Circuit Board, which is a common form of electronic hardware, in which electrical contacts and connecting wires are printed onto plastic substrate boards. Chips and discrete electronic components are also mounted on these boards, which provide a convenient, mass-producible means to wire together and package such discrete electronic components and devices.

As used herein, the term “PCB device” or “PCB EWOD device”, or “EWOD PCB device” refer to devices in which the PCB is printed with an array of electrodes, and wires routed to these electrodes, to form the array of electrodes for an EWOD array device, such that each electrode can be independently controlled, when the electrode control lines are further connected to suitable voltage supply hardware.

As used herein, the term “chip device” refers to a device that may be a CMOS integrated circuit semiconductor chip, or, when it makes sense in context, a TFT chip, such as TFT circuits fabricated on a glass or plastic substrate.

As used herein, the term “DNA” in various contexts may refer to single stranded or double stranded forms of the molecule. The term “DNA” may also in various contexts refers not only to strands composed of the four bases A, C, G, T, but also of ribonucleotides such as in RNA, other base analogues, such as U (uracil), I (Inosine), and other well-known universal bases or base analogues or modified or marked bases, including well-known epigenetics marks on bases, such as 5mC (5-methyl-C), as well as dye-labelled bases, or bases modified for future labelling or conjugation, such as biotinylated bases, or thiolate bases, and in general any other widely known modified forms of bases used in DNA oligonucleotides, including possible modifications in the sugar or backbone of DNA as well. In addition, where it makes sense in context, the term DNA encompasses other nucleic acid (NA) polymers such as RNA (Ribo-), PNA (Peptide-), LNA (Locked-), and diverse forms of XNA (Xeno-).

As used herein, the terms “DNA assembly” or “joining DNA” refers to any process for physically connecting together two or more existing DNA molecule strands, with the connection at or near the ends, to produce a single molecule strand. In various contexts, such strands may be entirely single stranded DNA, or entirely double stranded DNA, or DNA that is partially single stranded and double stranded. Such strands that are assemble or joined may be connected by covalent phosphate backbone bonds, or may be joined through the hydrogen bonding of complementary regions, or in some contexts may be joined through other chemical reactions and chemical groups, such as carbon chain linkers from the end of one backbone to the start of another.

As used herein, the term “nanochannel” refers to any channel structure that is nanometer (nm) scale in its width and depth dimensions, such as up to 10 nm, or up to 100 nm, or several hundred nm, and substantially longer in its third dimension of length, such as 1000 nm or longer, 10,000 nm or longer, or 100,000 nm or longer, or up to 1 millimeter (mm) or longer, or 10 mm or longer. Such channels may be straight, curved, or branched in various contexts. Such channels may reside in a single plane or may extend into 3D within a material substrate.

As used herein, the term “DNA”, as makes sense in context, may refer to the physical material of deoxyribonucleic acid, oligomers of such, or pools of such material, or alternatively to the symbolic sequences for such, in contexts where this makes sense. For the physical material, DNA, where it makes sense, can also refer to common chemical analogues of DNA or nucleic acid oligomers, such as RNA, or such as may contain modified bases, or analogues such as PNA (Peptide-) or LNA (Locked-) or XNA (Xeno-).

As used herein, “DNA sequencing” refers to processes for reading the identities of the series of bases in a DNA strand or strands.

As used herein, the term “PCR”, which is an acronym for Polymerase Chain Reaction, generally refers to any means of amplifying or copying DNA, including by thermo-cycling PCR, or isothermal PCR reactions, or generally any other processes that can be used to amplify or copy DNA.

As used herein, the term “MAGE” is an acronym for Multiplex Automated Genome Engineering, which refers to a known molecular biology method of engineering cell genomes, in which DNA oligos are serially introduced to the cell, to affect a series of genome edits on the genome of the target cell.

As used herein, the term “organoid” refers to a population of multiple cells, that has grown under chemical stimulation and from suitable progenitor cells in such a way that it has structural or functional characteristics associated with a certain organ. For example, a group of cardiomyocytes that have an electrical pulsation analogous to the heart beating, or a group of neurons forming a neural network that can transmit electrical signals throughout the neural network.

1-43. (canceled)

44. A system, comprising:
a digital microfluidic device configured to support first and second sets of droplets, wherein the first set of droplets comprises one or more payload molecules; and
wherein the second set of droplets comprises one or more expression systems; and
a plurality of electrodes configured to manipulate the first or
the second set of droplets.

45. The system of claim 44, wherein the first set of droplets is supported on a first portion of the digital microfluidic device, and the second set of droplets is supported on a second portion of the digital microfluidic device.

46. The system of claim 44, wherein being configured to manipulate the first set of droplets comprises being configured to move each droplet of the first set of droplets into proximity or merge with a corresponding droplet of the second set of droplets.

47. The system of claim 46, wherein the digital microfluidic device is configured to merge droplets on a massively parallel scale.

48. The system of claim 46, wherein the digital microfluidic device is configured to merge, in parallel between the first portion of the microfluidic electrowetting array device and the second portion of the microfluidic electrowetting array device, droplets at a scale of at least 10,000 droplets; at least 100,000 droplets; at least 1,000,000 droplets; at least 10,000,000 droplets; or more than 10,000,000 droplets.

49. The system of claim 46, wherein the first set of droplets and the second set of droplets are merged to form a set of merged droplets comprising one or more payload molecules and one or more cell packages, and wherein the system is configured to perform an insertion process so that at least one DNA payload within each merged droplet is functionally integrated with at least one cell package within the same merged droplet.

50. The system of claim 49, wherein the insertion process comprises one or more of: a mechanical injection, chemo-poration, electro-poration, providing heating or cooling to a droplet of the first and second set of droplets

51. The system of claim 44, wherein the digital microfluidic device is integrated for material transfer with a second chip device.

52. The system of claim 51, wherein the integration for material transfer is configured to transfer one or more materials selected from the group consisting of: solutions, payload molecules, chemical reagents, and cells.

53. The system of claim 51, wherein the integration for material transfer is configured for transferring materials iteratively between the digital microfluidic device and the second chip device.

54. The system of claim 51, wherein the second chip device comprises a DNA oligonucleotide synthesis or assembly device.

55. The system of claim 51, wherein the system further comprises a third chip device that is integrated for material transfer with the second chip device and the digital microfluidic device.

56. The system of claim wherein the expression systems comprise cell packages.

57. The system of claim 44, wherein the expression systems comprise cell-free expression droplets.

58. The system of claim 44, wherein the payload molecules comprise nucleic acids.
